(19) United States
(12) Patent Application Publication
(10) Pub. No.: US 2022/0233677 A1
(43) Pub. Date: Jul. 28, 2022

(54) METHODS FOR TREATING CANCERS WITH MODIFIED PBMCS

(71) Applicant: SQZ Biotechnologies Company, Watertown, MA (US)

(72) Inventors: Oliver ROSEN, Watertown, MA (US); Ruiru JI, Watertown, MA (US)

(21) Appl. No.: 17/563,866

(22) Filed: Dec. 28, 2021

Related U.S. Application Data
(60) Provisional application No. 63/278,788, filed on Nov. 12, 2021.
(60) Provisional application No. 63/249,739, filed on Sep. 29, 2021.
(60) Provisional application No. 63/190,194, filed on May 18, 2021.
(60) Provisional application No. 63/131,504, filed on Dec. 29, 2020.

(51) Int. Cl. A61K 39/12; A61K 39/395; A61K 47/42; A61K 39/39; A61P 35/00

(52) U.S. Cl. CPC A61K 39/12; A61K 39/3955; A61K 2039/5154; A61K 39/39; A61P 35/00; A61K 47/42

(57) ABSTRACT

The present application provides modified PBMCs for treating HPV-associated cancers. The modified PBMCs are derived from input PBMCs in which at least one HPV antigen has been delivered intracellularly. In some embodiments, the PBMCs are administered in combination with a checkpoint inhibitor such as a CTLA4 antagonist and/or a PD-1/PD-L1 agonist.

### CROSS REFERENCE TO RELATED APPLICATIONS

This application claims the benefit of U.S. Provisional Application No. 63/131,504, filed on Dec. 29, 2020, U.S. Provisional Application No. 63/190,194, filed on May 18, 2021, U.S. Provisional Application No. 63/249,739, filed on Sep. 29, 2021, and U.S. Provisional Application No. 63/278,788, filed on Nov. 12, 2021, the entire contents of each of which are incorporated herein by reference.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 750322003100SEQLIST.TXT, date recorded: Dec. 23, 2021, size: 13,144 bytes).

### FIELD OF THE INVENTION

The present disclosure relates generally to methods of using peripheral blood mononuclear cells (PBMCs) comprising one or more human papillomavirus (HPV) antigens for treating an individual with HPV-associated cancers, as well as doses and regimens thereof. Also disclosed are methods of manufacturing such PBMCs comprising the HPV antigen, and compositions thereof.

### BACKGROUND OF THE INVENTION

Papillomaviruses are small nonenveloped DNA viruses with a virion size of ˜55 nm in diameter. More than 100 HPV genotypes are completely characterized, and a higher number is presumed to exist. HPV is a known cause of cervical cancers, as well as some vulvar, vaginal, penile, oropharyngeal, anal, and rectal cancers. Although most HPV infections are asymptomatic and clear spontaneously, persistent infections with one of the oncogenic HPV types can progress to precancer or cancer. Other HPV-associated diseases can include common warts, plantar warts, flat warts, anogenital warts, anal lesions, epidermodysplasia, focal epithelial hyperplasia, mouth papillomas, verrucous cysts, laryngeal papillomatosis, squamous intraepithelial lesions (SILs), cervical intraepithelial neoplasia (CIN), vulvar intraepithelial neoplasia (VIN) and vaginal intraepithelial neoplasia (VAIN).

Many of the known HPV types cause benign lesions with a subset being oncogenic. Based on epidemiologic and phylogenetic relationships, HPV types are classified into fifteen “high-risk types” (HPV 16, 18, 31, 33, 35, 39, 45, 51, 52, 56, 58, 59, 68, 73, and 82) and three “probable high-risk types” (HPV 26, 53, and 66), which together are known to manifest as low and high grade cervical changes and cancers, as well as other anogenital cancers such as vulval, vaginal, penile, anal, and perianal cancer, as well as head and neck cancers. Recently, the association of high-risk types HPV 16 and 18 with breast cancer was also described. Eleven HPV types classified as “low-risk types” (HPV 6, 11, 40, 42, 43, 44, 54, 61, 70, 72, and 81) are known to manifest as benign low-grade cervical changes, genital warts and recurrent respiratory papillomatosis. Cutaneous HPV types 5, 8, and 92 are associated with skin cancer. In some HPV-associated cancers, the immune system is depressed and correspondingly, the antitumor response is significantly impaired. See Suresh and Burtness Am J Hematol Oncol 13(6):20-27 (2017).

Immunotherapy can be divided generally into two main types of interventions, either passive or active. Passive protocols include administration of pre-activated and/or engineered cells (e.g., CAR T cells), disease-specific therapeutic antibodies, and/or cytokines. Active immunotherapy strategies are directed at stimulating immune system effector functions in vivo. Several current active protocols include vaccination strategies with disease-associated peptides, lysates, or allogeneic whole cells, infusion of autologous dendritic cell (DCs) as vehicles for tumor antigen delivery, and infusion of immune checkpoint modulators. See Papaioannou, Nikos E., et al. Annals of translational medicine 4.14 (2016). Adoptive immunotherapy can be employed to modulate the immune response, enhance antitumor activity, and achieve the goal of treating or preventing HPV-associated cancers.

CD8⁺ cytotoxic T lymphocytes (CTL) and CD4⁺ helper T (Th) cells stimulated by disease-associated antigens have the potential to target and destroy diseased cells; however, current methods for inducing endogenous T cell responses have faced challenges. The disclosure herein also includes methods, treatments, doses and regimens for treating individuals with HPV-associated cancers using PBMCs comprising HPV antigens. Also provided are methods used to efficiently generate PBMCs comprising HPV antigens and/or adjuvants in a high throughput manner, which can be utilized in inducing a robust T cell response to HPV antigens.

All references cited herein, including patent applications and publications, are incorporated by reference in their entirety. The patent publications WO 2013/059343, WO 2015/023982, WO 2016/070136, WO2017041050, WO2017008063, WO 2017/192785, WO 2017/192786, WO 2019/178005, WO 2019/178006, WO 2020/072833, WO 2020/154696, and WO 2020/176789, US 20180142198, and US 20180201889 are hereby expressly incorporated by reference in their entirety.

### BRIEF SUMMARY OF THE INVENTION

In some aspects, the invention provides methods for treating a human papilloma virus (HPV)-associated cancer in an individual, the method comprising: administering an effective amount of a composition comprising peripheral blood mononuclear cells (PBMCs) to the individual, wherein the PBMCs comprise at least one HPV antigen delivered intracellularly, and administering an effective amount of an antagonist of CTLA-4 and/or an antagonist of PD-1/PD-L1 to the individual. In some embodiments, the antagonist of CTLA4 is an antibody that binds CTLA4. In some embodiments, the antagonist of PD-1/PD-L1 is an antibody that binds PD-1 or an antibody that binds PD-L1. In some embodiments, an antibody that binds CTLA-4 and an antibody that binds PD-1 are administered to the individual. In some embodiments, an antibody that binds CTLA-4 is administered to the individual and an antibody that binds PD-L1 is administered to the individual. In some embodiments, the antibody that binds CTLA-4 is ipilimumab. In some embodiments, the antibody that binds PD-1 is nivolumab. In some embodiments, the antibody that binds PD-1 is pembrolizumab. In some embodiments, the antibody that binds PD-L1 is atezolizumab.

In some aspects, the invention provides methods for treating a HPV+ recurrent, locally advanced or metastatic tumor in an individual, the method comprising administering an effective amount of a composition comprising peripheral blood mononuclear cells (PBMCs) to the individual, wherein the PBMCs comprise at least one HPV antigen delivered intracellularly. In some embodiments, the composition comprising PBMCs is administered in conjunction with one or more immune checkpoint inhibitors. In some embodiments, the checkpoint inhibitor is an antagonist of CTLA-4 and/or an antagonist of PD-1/PD-L1 to the individual. In some embodiments, the one or more immune checkpoint inhibitor is an antibody that binds PD-L1, CTLA-4, or PD-1. In some embodiments, the composition comprising PBMCs is administered in conjunction with an antibody that binds CTLA-4 and an antibody that binds PD-1. In some embodiments, the antibody that binds PD-L1 is atezolizumab. In some embodiments, the antibody that binds CTLA-4 is ipilimumab. In some embodiments, the antibody that binds PD-1 is nivolumab. In some embodiments, the antibody that binds PD-1 is pembrolizumab.

In some embodiments of the invention, PBMCs of the invention comprise at least one HPV antigen wherein the one HPV antigen is an HPV-16 antigen or an HPV-18 antigen. In some embodiments, the at least one HPV antigen comprises a peptide derived from HPV E6 and/or E7. In some embodiments, the at least one HPV antigen comprises an HLA-A2-restricted peptide derived from HPV E6 and/or E7. In some embodiments, the HLA-A2-restricted peptide comprises the amino acid sequence of any one of SEQ ID NOs:1-4. In some embodiments, the at least one HPV antigen comprises the amino acid sequence of any one of SEQ ID NOs:18-25. In some embodiments, the PBMCs comprise an antigen comprising the amino acid sequence of SEQ ID NO:19 and an antigen comprising the amino acid sequence of SEQ ID NO:23.

In some embodiments of the methods of treatment of the invention, the individual is human. In some embodiments, the individual is positive for HLA-A*02. In some embodiments, the PBMCs are positive for HLA-A*02. In some embodiments, the PBMCs are autologous to the individual. In some embodiments, the individual is positive for human immunodeficiency virus (HIV). In some embodiments, the HPV-associated cancer is head and neck cancer, cervical cancer, anal cancer or esophageal cancer.

In some embodiments, the composition comprising PBMCs are administered intravenously. In some embodiments, the antagonist of CTLA-4 and/or antagonist of PD-1/PD-La is administered intravenously, orally, or subcutaneously. In some embodiments, the antibody that binds CTLA-4 and/or the antibody that binds PD-1 and/or the antibody that binds PD-L1 is administered intravenously.

In some embodiments of the method of treatment of the invention, the effective amount of PBMCs comprising the at least one HPV antigen is about 0.5×10⁶ cells/kg to about 5.0×10⁶ cells/kg. In some embodiments, the effective amount of ipilimumab is about 1 mg/kg to about 3 mg/kg. In some embodiments, the effective amount of nivolumab is about 360 mg. In some embodiments, the effective amount of atezolizumab is about 1200 mg. In some embodiments, the composition comprising the PBMCs is delivered on day 1 of a three-week cycle. In some embodiments, the composition comprising the PBMCs is further administered on day 2 of a first three-week cycle. In some embodiments, about 0.5×10⁶ cells/kg, about 2.5×10⁶ cells/kg about 5.0×10⁶ cells/kg are administered on day 1 of each three-week cycle. In some embodiments, about 0.5×10⁶ cells/kg, about 2.5×10⁶ cells/kg or about 5.0×10⁶ cells/kg are administered on day 2 of the first three-week cycle. In some embodiments, the antibody that binds CTLA 4 and/or the antibody that binds PD-1 and/or the antibody that binds PD-L1 is administered once per three-week cycle. In some embodiments, the antibody that binds CTLA-4 is administered on day 1 of each three-week cycle. In some embodiments, the antibody that binds CTLA-4 is administered once per two three-week cycles. In some embodiments, the antibody that binds CTLA-4 is ipilimumab, wherein the ipilimumab is administered at a dose of about 3 mg/kg. In some embodiments, the antibody that binds PD-1 is administered on day 8 of the first three-week cycle and day 1 of each subsequent cycle. In some embodiments, the antibody that binds PD-1 is nivolumab, wherein the nivolumab is administered at a dose of about 360 mg. In some embodiments, the antibody that binds CTLA-4 is ipilimumab, wherein the ipilimumab is administered on day 1 of the first three-week cycle of two three-week cycles at a dose of about 1 mg/kg and the antibody that binds PD-1 is administered on day 8 of the first three-week cycle and day 1 of each subsequent cycle at a dose of about 360 mg. In some embodiments, the antibody that binds PD-L1 is administered on day 8 of the first three-week cycle and day 1 of each subsequent cycle. In some embodiments, the antibody that binds PD-L1 is atezolizumab, wherein the atezolizumab is administered at a dose of about 1200 mg. In some embodiments, the composition comprising PBMCs is administered to the individual for at least about three months, six months, nine months or one year.

In some embodiments of the invention, the composition comprising PBMCs to be administered to the individual comprises a) about 5×10⁶ PBMCs to about 5×10⁷ PBMCs, b) cryopreservation medium at a percentage of about 40% to about 60% (w/w), c) hypothermic preservation medium at a percentage of about 25% to about 35% (w/w), and d) human serum albumin about 3% to about 8% (w/w), wherein the pH of the formulation is about pH 6.0 to about pH 8.5. In some embodiments, the composition comprising PBMCs comprises a) about 1×10⁶ PBMCs/mL to about 1×10⁷ PBMCs/mL, b) cryopreservation medium at a at a percentage of about 40% to about 60% (w/w), c) hypothermic preservation medium at a percentage of about 25% to about 35% (w/w), and d) human serum albumin at a percentage of about 3% to about 8% (w/w), wherein the pH of the formulation is about pH 6.0 to about pH 8.5. In some embodiments, the composition comprising PBMCs comprises a) about 2.75×10⁷ PBMCs, b) cryopreservation medium at a percentage of about 50% (w/w), c) hypothermic preservation medium at a percentage of about 30% (w/w), and d) human serum albumin at a percentage of about 5% (w/w), wherein the pH of the formulation is about pH 7.4. In some embodiments, the composition comprising PBMCs comprises a) about 5×10⁶ PBMCs/mL, b) cryopreservation medium at a percentage of about 50% (w/w), c) hypothermic preservation medium at a percentage of about 30% (w/w), and d) human serum albumin at a percentage of about 5% (w/w), wherein the pH of the formulation is about pH 7.4. In some embodiments, the composition comprising PBMCs comprises a) about 5×10⁶ PBMCs to about 5×10⁷ PBMCs, b) cryopreservation medium at a percentage of about 65% to about 95% (w/w), c) human serum albumin at a percentage of about 3% to about 8% (w/w), wherein the pH of the formulation is about pH 6.0 to about pH 8.5. In some embodiments, the composition comprising PBMCs comprises a) about 1×10⁶ PBMCs/mL to about 1×10⁷ PBMCs/mL, b) cryopreservation medium at a percentage of about 65% to about 95% (w/w), c) human serum albumin at a percentage of about 3% to about 8% (w/w), wherein the pH of the formulation is about pH 6.0 to about pH 8.5. In some embodiments, the composition comprising PBMCs comprises a) about 2.5×10⁷ PBMCs, b) cryopreservation medium at a percentage of about 80% (w/w), c) human serum albumin at a percentage of about 5% (w/w), wherein the pH of the formulation is about pH 7.4. In some embodiments, the composition comprising PBMCs comprises a) about 5×10⁶ PBMCs/mL, b) cryopreservation medium at a percentage of about 80% (w/w), c) human serum albumin at a percentage of about 5% (w/w), wherein the pH of the formulation is about pH 7.4. In some embodiments, the cryopreservation medium is CryoStor® CS10. In some embodiments, the hypothermic preservation medium is HypoThermasol® FRS.

In some embodiments, the PBMCs of the invention comprises two or more of T cells, B cells, NK cells or monocytes. In some embodiments, the PBMCs comprises T cells, B cells, NK cells and monocytes. In some embodiments, (a) about 25% to about 80% of the PBMCs are T cells; (b) about 1.5% to about 30% of the PBMCs are B cells; (c) about 3.0% to about 20% of the PBMCs are NK cells; or (d) about 4.0% to about 45% of the PBMCs are monocytes.

In some embodiments of the invention, the PBMCs comprising the at least one HPV antigen are prepared by a process comprising: a) passing a cell suspension comprising a population of input PBMCs through a cell-deforming constriction, wherein a diameter of the constriction is a function of a diameter of the input PBMCs in the suspension, thereby causing perturbations of the input PBMCs large enough for the at least one HPV antigen to pass through to form perturbed input PBMCs; and b) incubating the population of perturbed input PBMCs with the at least one HPV antigen for a sufficient time to allow the antigen to enter the perturbed input PBMCs, thereby generating the PBMCs comprising the at least one HPV antigen. In some embodiments, the diameter of the constriction is about 4.2 μm to about 6 μm or about 4.2 μm to about 4.8 μm. In some embodiments, the PBMCs comprising the at least one HPV antigen are conditioned. In some embodiments, the PBMCs comprising the at least one HPV antigen are conditioned by a process comprising incubating the PBMCs with an adjuvant for about 2 hours to about 10 hours, about 3 hours to about 6 hours, or about 4 hours at about 37° C. for the PBMCs to condition. In some embodiments, the adjuvant is a CpG oligodeoxynucleotide (ODN), LPS, IFN-α, STING agonists, RIG-I agonists, poly I:C, R837, R848, a TLR3 agonist, a TLR4 agonist or a TLR 9 agonist. In some embodiments, the adjuvant is a CpG 7909 oligodeoxynucleotide (ODN).

### BRIEF DESCRIPTION OF THE DRAWINGS

FIG. 1 shows the treatment regime for cohorts 1-3.

FIG. 2 shows the treatment regime for cohort 4.

FIG. 3 shows the treatment regime for cohort 5.

FIG. 4 shows the treatment regime for cohort 6.

FIG. 5 shows the treatment regime for cohort 7.

FIG. 6 is a schematic showing the mechanism of SQZ-PBMC-HPV-101 investigational product generated by Cell Squeeze® technology, and showing that SQZ-PBMC-HPV vaccines directly stimulate CD8 T cell response.

FIG. 7 is a schematic of the clinical study design. SQZ-PBMC-HPV are either administered as monotherapy at the indicated dose (cells per body weight) every three weeks (q3w) in the monotherapy phase, or administered in combination with the indicated dose of checkpoint inhibitors (Atezolizumab, Ipilimumab (Ipi), Nivolumab (Nivo)) at the indicated intervals (q3w, q3w×4, q6w).

FIGS. 8A-8C show the manufacturing outcome for SQZ-PBMC-HPV, illustrating the average viability, the average end-to-end process time, and the IFN-γ secretion assay of SQZ-PBMC-HPV respectively.

FIG. 9 shows the summary of best overall response (BOR), Survival on study (Days) and Royal Marsden Hospital (RMH)1 Score across all cohorts.

FIGS. 10A-10C show the aggregate tumor size, the IHC image analyses showing change in CD8 TIL in the central tumor, and representative IHC images demonstrating CD8 TIL, respectively, in case study patient 2 after treatment.

FIGS. 11A-11C show the aggregate tumor size, the IHC image analyses showing change in CD8 TIL in the central tumor, and representative IHC images demonstrating CD8 TIL, respectively, in case study patient 7 after treatment.

FIG. 12 shows the density of CD8+ cells in tumors at screening (Pre) and at Cycle 2 Day 8 (C2D8, Post) for patients in cohorts 1, 2, 3 and 3a.

FIG. 13 shows the density of CD8+/granzyme B+(GZMB+) cells in tumors at screening (Pre) and C2D8 (Post) for patients in cohorts 1, 2, 3 and 3a.

FIG. 14 shows the density of CD8+/Ki67+ cells in tumors at screening (Pre) and C2D8 (Post) for patients in cohorts 1, 2, 3 and 3a.

FIG. 15 shows the density of CD8+/Ki67− cells in tumors at screening (Pre) and C2D8 (Post) for patients in cohorts 1, 2, 3 and 3a.

FIG. 16 shows the expression of MHC-1 in tumors at screening (Pre) and C2D8 (Post) for patients in cohorts 1, 2, 3 and 3a as measured by H-Score (top panel). The relative ratio of MHC-, MHC-1 low, MHC-1 medium, and MHC-1 high cells at screening (Pre) and C2D8 (Post) is shown in the lower panel.

FIG. 17 shows % of tumors cells with PD-L1 membrane staining at screening (Pre) and C2D8 (Post) for patients in cohorts 1, 2, 3 and 3a. TPS denoted tumor proportion score.

FIG. 18 shows the expression of HPV16 E6 in tumors at screening (Pre) and C2D8 (Post) for patients in cohorts 1, 2, 3 and 3a as measured by RNA ISH Modified H-Score (top panel). The relative ratio of HPV16 negative, HPV 16+1, HPV 16+2, HPV 16+3, and HPV 16+4, cells at screening (Pre) and C2D8 (Post) is shown in the lower panel. * denotes that the morphology of the cells were not suitable to be scored.

FIG. 19 shows the expression of HPV16 E7 in tumors at screening (Pre) and C2D8 (Post) for patients in cohorts 1, 2, 3 and 3a as measured by RNA ISH Modified H-Score (top panel). The relative ratio of HPV16 negative, HPV 16+1, HPV 16+2, HPV 16+3, and HPV 16+4, cells at screening (Pre) and C2D8 (Post) is shown in the lower panel. * denotes that the morphology of the cells were not suitable to be scored.

FIG. 20 shows % of tumors cells with PD-1 cells by area within the tumor center at screening (Pre) and C2D8 (Post) for patients in cohorts 1, 2, 3 and 3a. * denotes that the morphology of the cells were not suitable to be scored.

FIG. 21 shows CD8 infiltration of a tumor for patient 112-068 at screening (Pre) and at C2D8 (Post). Left panel shows tumor infiltration in the central tumor (CN) as well as infiltration in the stroma and parenchyma. Middle panel shows CTL, Treg and NK functionality in tumors as measured by CD8, GZMB and FoxP3. Right panel shows the percentage of CD8+ cells that are GZMB+ cells.

FIG. 22 shows examples of immunohistochemistry images from which the data in FIG. 21 was derived.

FIG. 23 shows the proliferating/activated cell density in a tumor from patient 112-068 at screening (Pre) and at C2D8 (Post) as shown by the density of cells that are CD8+, CD8+/Ki67−, CD8−/Ki67+ and CD8+/Ki67+. Immunohistochemistry is shown in right panel. Top images are low resolution, bottom images are higher resolution.

FIG. 24 shows the % of tumors cells from patient 112-068 with PD-L1 staining at screening (Pre) and at C2D8 (Post).

FIG. 25 shows expression of MHC-1 and HPV16 E6 and E7 epitopes in a tumor from patient 112-068 at screening (Pre) and at C2D8 (Post). Top left panel shows MHC-1 expression at screening and C2D8. Top middle panel shows the relative expression of MHC-1 in cells in the tumor at screening and C2D8. Bottom left panel shows HPV16 E6 expression. Bottom middle panel shows HPV16 E7 expression. Right panel shows examples of immunohistochemistry from which the data in the left and middle panels was obtained.

FIG. 26 shows tumor growth kinetics of patient 112-068.

FIG. 27 shows CD8 infiltration of a tumor for patient 103-027 at screening (Pre) and at C2D8 (Post). Left panel shows tumor infiltration in the central tumor (CN) as well as infiltration in the stroma and parenchyma. Middle panel shows CTL, Treg and NK functionality in tumors as measured by CD8, GZMB and FoxP3. Right panel shows the percentage of CD8+ cells that are GZMB+ cells.

FIG. 28 shows examples of immunohistochemistry images from which the date in FIG. 27 was derived.

FIG. 29 shows the proliferating/activated cell density in a tumor from patient 103-027 at screening (Pre) and at C2D8 (Post) as shown by the density of cells that are CD8+, CD8+/Ki67−, CD8−/Ki67+ and CD8+/Ki67+. Immunohistochemistry is shown in right panel. Top images are low resolution, bottom images are higher resolution.

FIG. 30 shows the % of tumors cells from patient 103-027 with PD-L1 staining at screening (Pre) and at C2D8 (Post).

FIG. 31 shows expression of MHC-1 and HPV16 E6 and E7 epitopes in a tumor from patient 103-027 at screening (Pre) and at C2D8 (Post). Top left panel shows MHC-1 expression at screening and C2D8. Top middle panel shows the relative expression of MHC-1 in cells in the tumor at screening and C2D8. Bottom left panel shows HPV16 E6 expression. Bottom middle panel shows HPV16 E7 expression. * denotes that the morphology of the cells were not suitable to be scored. Right panel shows examples of immunohistochemistry from which the data in the left and middle panels was obtained.

FIG. 32 shows CD8 infiltration of a tumor for patient 103-008 at screening (Pre) and at C2D8 (Post). Left panel shows tumor infiltration in the central tumor (CN) as well as infiltration in the stroma and parenchyma. Middle panel shows CTL, Treg and NK functionality in tumors as measured by CD8, GZMB and FoxP3. Right panel shows the percentage of CD8+ cells that are GZMB+ cells.

FIG. 33 shows examples of immunohistochemistry from which the date in FIG. 32 was derived.

FIG. 34 shows the proliferating/activated cell density in a tumor from patient 103-008 at screening (Pre) and at C2D8 (Post) as shown by the density of cells that are CD8+, CD8+/Ki67-, CD8-/Ki67+ and CD8+/Ki67+. Immunohistochemistry is shown in right panel. Top images are low resolution, bottom images are higher resolution.

FIG. 35 shows the % of tumors cells from patient 103-008 with PD-L1 staining at screening (Pre) and at C2D8 (Post).

FIG. 36 shows expression of MHC-1 and HPV16 E6 and E7 epitopes in a tumor from patient 103-008 at screening (Pre) and at C2D8 (Post). Top left panel shows MHC-1 expression at screening and C2D8. Top middle panel shows the relative expression of MHC-1 in cells in the tumor at screening and C2D8. Bottom left panel shows HPV16 E6 expression. Bottom middle panel shows HPV16 E7 expression. Right panel shows examples of immunohistochemistry from which the data in the left and middle panels was obtained.

The patent or application file contains at least one drawing executed in color. Copies of this patent or patent application publication with color drawing(s) will be provided by the Office upon request and payment of the necessary fee.

### DETAILED DESCRIPTION OF THE INVENTION

In some aspects, the present invention provides methods for treating a human papilloma virus (HPV)-associated cancer in an individual, the method comprising administering an effective amount of a composition comprising peripheral blood mononuclear cells (PBMCs) to the individual, wherein the PBMCs comprise at least one HPV antigen delivered intracellularly.

In some aspects, the present invention provides methods for treating a HPV-associated cancer in an individual, the method comprising administering an effective amount of a composition comprising PBMCs to the individual, wherein the PBMCs comprise at least one HPV antigen delivered intracellularly, and administering an effective amount of one or more immune checkpoint inhibitors. In some embodiments the one or more immune checkpoint inhibitors comprise an antagonist of CTLA-4 (such as but not limited to ipilimumab), an antagonist of PD-1 (such as but not limited to nivolumab), and/or an antagonist of PD-L1 (such as but not limited to atezolizumab).

In some aspects, the present invention provides methods for treating a HPV-associated cancer in an individual, the method comprising administering an effective amount of a composition comprising activated PBMCs to the individual, wherein the PBMCs comprise at least one HPV antigen delivered intracellularly, and administering an effective amount of one or more of ipilimumab, nivolumab, or atezolizumab, wherein the PBMCs comprising the HPV antigen, and/or the one or more immune checkpoint inhibitors are administered in three-week cycles, wherein effective amount of PBMCs is about 0.5×10⁶ cells/kg to about 5×10⁶ cells/kg, wherein the effective amount of ipilimumab is about 1 mg/kg to about 3 mg/kg, wherein the effective amount of nivolumab is about 360 mg, and wherein the effective amount of atezolizumab is about 1200 mg.

Also provided are compositions of PBMCs comprising the HPV antigen and adjuvant, and the methods of preparing the PBMCs comprising the HPV antigen and adjuvant. In some embodiments, the PBMCs are prepared by a process comprising: a) passing a cell suspension comprising a population of input PBMCs through a cell-deforming constriction, wherein a diameter of the constriction is a function of a diameter of the PBMCs in the suspension, thereby causing perturbations of the input PBMCs large enough for the HPV antigen and the adjuvant to pass through to form perturbed input PBMCs; and b) incubating the population of perturbed input PBMCs with the HPV antigen and the adjuvant for a sufficient time to allow the antigen to enter the perturbed input PBMCs, thereby generating the modified PBMCs comprising the HPV antigen and the adjuvant. Also provided are compositions for use in inducing an immune response to HPV antigens or for treating a HPV-associated cancer. Also provided are uses of a composition comprising an effective amount of the PBMCs in the manufacture of a medicament for stimulating an immune response to a HPV antigen or for treating a HPV-associated cancer.

### General Techniques

The techniques and procedures described or referenced herein are generally well understood and commonly employed using conventional methodology by those skilled in the art, such as, for example, the widely utilized methodologies described in Molecular Cloning: A Laboratory Manual (Sambrook et al., 4^{th} ed., Cold Spring Harbor Laboratory Press, Cold Spring Harbor, N.Y., 2012); Current Protocols in Molecular Biology (F. M. Ausubel, et al. eds., 2003); the series Methods in Enzymology (Academic Press, Inc.); PCR 2: A Practical Approach (M. J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R. I. Freshney, 6^{th} ed., J. Wiley and Sons, 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C. A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 2011)

### Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

As used herein, the singular form “a”, “an”, and “the” includes plural references unless indicated otherwise.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a “peripheral blood mononuclear cells” or “PBMCs” refers to a heterogeneous population of blood cells having a round nucleus. Examples of cells that may be found in a population of PBMCs include lymphocytes such as T cells, B cells, NK cells (including natural killer T cells (NKT cells) and cytokine-induced killer cells (CIK cells)) and monocytes such as macrophages and dendritic cells. A “plurality of PBMCs” as used herein refers to a preparation of PBMCs comprising cells of at least two types of blood cells. In some embodiments, a plurality of PBMCs comprises two or more of T cells, B cells, NK cells, macrophages or dendritic cells. In some embodiments, a plurality of PBMCs comprises three or more of T cells, B cells, NK cells, macrophages or dendritic cells. In some embodiments, a plurality of PBMCs comprises four or more of T cells, B cells, NK cells, macrophages or dendritic cells. In some embodiments, a plurality of PBMCs comprises T cells, B cells, NK cells, macrophages and dendritic cells.

PBMCs can be isolated by means known in the art. For example, PBMCs can be derived from peripheral blood of an individual based on density of PBMCs compared to other blood cells. In some embodiments, PBMCs are derived from peripheral blood of an individual using Ficoll (e.g., a Ficoll gradient). In some embodiments, PBMCs are derived from peripheral blood of an individual using ELUTRA® cell separation system. PBMCs can be obtained from an individual undergoing apheresis.

In some embodiments, a population of PBMCs is isolated from an individual. In some embodiments, a plurality of PBMCs is an autologous population of PBMCs where the population is derived from a particular individual, manipulated by any of the methods described herein, and returned to the particular individual. In some embodiments, a plurality of PBMCs is an allogeneic population of PBMCs where the population is derived from one individual, manipulated by any of the methods described herein, and administered to a second individual.

In some embodiments, a plurality of PBMCs is a reconstituted preparation of PBMCs. In some embodiments, the plurality of PBMCs may be generated by mixing cells typically found in a population of PBMCs; for example, by mixing populations of two or more of T cells, B cells, NK cells, or monocytes.

As used herein “payload” refers to the material that is being delivered into, such as loaded in, the PBMCs. “Payload,” “cargo,” “delivery material,” and “compound” are used interchangeably herein. In some embodiments, a payload may refer to a protein, a small molecule, a nucleic acid (e.g., RNA and/or DNA), a lipid, a carbohydrate, a macromolecule, a vitamin, a polymer, fluorescent dyes and fluorophores, carbon nanotubes, quantum dots, nanoparticles, and steroids. In some embodiments, the payload may refer to a protein or small molecule drug. In some embodiments, the payload may comprise one or more compounds.

The term “heterologous” as it relates to nucleic acid sequences such as coding sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention. Allelic variation or naturally occurring mutational events do not give rise to heterologous DNA, as used herein.

The term “heterologous” as it relates to amino acid sequences such as peptide sequences and polypeptide sequences, denotes sequences that are not normally joined together, and/or are not normally associated with a particular cell. Thus, a “heterologous” region of a peptide sequence is a segment of amino acids within or attached to another amino acid molecule that is not found in association with the other molecule in nature. For example, a heterologous region of a peptide construct could include the amino acid sequence of the peptide flanked by sequences not found in association with the amino acid sequence of the peptide in nature. Another example of a heterologous peptide sequence is a construct where the peptide sequence itself is not found in nature (e.g., synthetic sequences having amino acids different as coded from the native gene). Similarly, a cell transformed with a vector that expresses an amino acid construct which is not normally present in the cell would be considered heterologous for purposes of this invention. Allelic variation or naturally occurring mutational events do not give rise to heterologous peptides, as used herein.

The term “exogenous” when used in reference to an agent, such as an antigen or an adjuvant, with relation to a cell refers to an agent outside of the cell or an agent delivered into the cell from outside the cell. The cell may or may not have the agent already present, and may or may not produce the agent after the exogenous agent has been delivered.

The term “homologous” as used herein refers to a molecule which is derived from the same organism. In some examples the term refers to a nucleic acid or protein which is normally found or expressed within the given organism.

As used herein, “treatment” or “treating” is an approach for obtaining beneficial or desired results, including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the invention contemplate any one or more of these aspects of treatment.

As used herein, the term “prophylactic treatment” refers to treatment, wherein an individual is known or suspected to have or be at risk for having a disorder but has displayed no symptoms or minimal symptoms of the disorder. An individual undergoing prophylactic treatment may be treated prior to onset of symptoms. In some embodiments, an individual may be treated if they have a precancerous lesion, particularly a precancerous lesion associated with HPV infection.

As used herein, by “combination therapy” is meant that a first agent be administered in conjunction with another agent. “In conjunction with” refers to administration of one treatment modality in addition to another treatment modality, such as administration of a composition of PMBCs as described herein in addition to administration of an immunoconjugate as described herein to the same individual. As such, “in conjunction with” refers to administration of one treatment modality before, during, or after delivery of the other treatment modality to the individual.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

In the context of cancer, the term “treating” includes any or all of killing cancer cells, inhibiting growth of cancer cells, inhibiting replication of cancer cells, lessening of overall tumor burden and ameliorating one or more symptoms associated with the disease.

As used herein, the term “modulate” may refer to the act of changing, altering, varying, or otherwise modifying the presence, or an activity of, a particular target. For example, modulating an immune response may refer to any act leading to changing, altering, varying, or otherwise modifying an immune response. In some examples, “modulate” refers to enhancing the presence or activity of a particular target. In some examples, “modulate” refers to suppressing the presence or activity of a particular target. In other examples, modulating the expression of a nucleic acid may include, but not limited to a change in the transcription of a nucleic acid, a change in mRNA abundance (e.g., increasing mRNA transcription), a corresponding change in degradation of mRNA, a change in mRNA translation, and so forth.

As used herein, the term “inhibit” may refer to the act of blocking, reducing, eliminating, or otherwise antagonizing the presence, or an activity of, a particular target. Inhibition may refer to partial inhibition or complete inhibition. For example, inhibiting an immune response may refer to any act leading to a blockade, reduction, elimination, or any other antagonism of an immune response. In other examples, inhibition of the expression of a nucleic acid may include, but not limited to reduction in the transcription of a nucleic acid, reduction of mRNA abundance (e.g., silencing mRNA transcription), degradation of mRNA, inhibition of mRNA translation, gene editing and so forth. In other examples, inhibition of the expression of a protein may include, but not be limited to, reduction in the transcription of a nucleic acid encoding the protein, reduction in the stability of mRNA encoding the protein, inhibition of translation of the protein, reduction in stability of the protein, and so forth. In another example, inhibit may refer to the act of slowing or stopping growth; for example, retarding or preventing the growth of a tumor cell.

As used herein, the term “suppress” may refer to the act of decreasing, reducing, prohibiting, limiting, lessening, or otherwise diminishing the presence, or an activity of, a particular target. Suppression may refer to partial suppression or complete suppression. For example, suppressing an immune response may refer to any act leading to decreasing, reducing, prohibiting, limiting, lessening, or otherwise diminishing an immune response. In other examples, suppression of the expression of a nucleic acid may include, but not limited to reduction in the transcription of a nucleic acid, reduction of mRNA abundance (e.g., silencing mRNA transcription), degradation of mRNA, inhibition of mRNA translation, and so forth. In other examples, suppression of the expression of a protein may include, but not be limited to, reduction in the transcription of a nucleic acid encoding the protein, reduction in the stability of mRNA encoding the protein, inhibition of translation of the protein, reduction in stability of the protein, and so forth.

As used herein, the term “enhance” may refer to the act of improving, boosting, heightening, or otherwise increasing the presence, or an activity of, a particular target. For example, enhancing an immune response may refer to any act leading to improving, boosting, heightening, or otherwise increasing an immune response. In one exemplary example, enhancing an immune response may refer to employing an antigen and/or adjuvant to improve, boost, heighten, or otherwise increase an immune response. In other examples, enhancing the expression of a nucleic acid may include, but not limited to increase in the transcription of a nucleic acid, increase in mRNA abundance (e.g., increasing mRNA transcription), decrease in degradation of mRNA, increase in mRNA translation, and so forth. In other examples, enhancing the expression of a protein may include, but not be limited to, increase in the transcription of a nucleic acid encoding the protein, increase in the stability of mRNA encoding the protein, increase in translation of the protein, increase in the stability of the protein, and so forth.

As used herein, the term “induce” may refer to the act of initiating, prompting, stimulating, establishing, or otherwise producing a result. For example, inducing an immune response may refer to any act leading to initiating, prompting, stimulating, establishing, or otherwise producing a desired immune response. In other examples, inducing the expression of a nucleic acid may include, but not limited to initiation of the transcription of a nucleic acid, initiation of mRNA translation, and so forth. In other examples, inducing the expression of a protein may include, but not be limited to, increase in the transcription of a nucleic acid encoding the protein, increase in the stability of mRNA encoding the protein, increase in translation of the protein, increase in the stability of the protein, and so forth.

The term “polynucleotide” or “nucleic acid” as used herein refers to a polymeric form of nucleotides of any length, including ribonucleotides and deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. The backbone of the polynucleotide can comprise repeating units, such as N-(2-aminoethyl)-glycine, linked by peptide bonds (i.e., peptide nucleic acid). Alternatively, the backbone of the polynucleotide can comprise a polymer of synthetic subunits such as phosphoramidates and phorphorthioates and thus can be an oligodeoxynucleoside phosphoramidate (P-NH2) or a mixed phosphorothioate-phosphorodiester oligomer or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

As used herein, the term “adjuvant” refers to a substance which modulates and/or engenders an immune response. Generally, the adjuvant is administered in conjunction with an antigen to effect enhancement of an immune response to the antigen as compared to antigen alone. Various adjuvants are described herein.

The terms “CpG oligodeoxynucleotide” and “CpG ODN” herein refer to DNA molecules of 10 to 30 nucleotides in length containing a dinucleotide of cytosine and guanine separated by a phosphate (also referred to herein as a “CpG” dinucleotide, or “CpG”). The CpG ODNs of the present disclosure contain at least one unmethylated CpG dinucleotide. That is, the cytosine in the CpG dinucleotide is not methylated (i.e., is not 5-methylcytosine). CpG ODNs may have a partial or complete phosphorothioate (PS) backbone.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug Administration.

For any of the structural and functional characteristics described herein, methods of determining these characteristics are known in the art.

As used herein, “microfluidic systems” refers to systems in which low volumes (e.g., mL, nL, pL, fL) of fluids are processed to achieve the discrete treatment of small volumes of liquids. Certain implementations described herein include multiplexing, automation, and high throughput screening. The fluids (e.g., a buffer, a solution, a payload-containing solution, or a cell suspension) can be moved, mixed, separated, or otherwise processed. In certain embodiments described herein, microfluidic systems are used to apply mechanical constriction to a cell suspended in a buffer, inducing perturbations in the cell (e.g., holes) that allow a payload or compound to enter the cytosol of the cell.

As used herein, a “constriction” may refer to a portion of a microfluidic channel defined by an entrance portion, a center point, and an exit portion, wherein the center point is defined by a width, a length, and a depth. In other examples, a constriction may refer to a pore or may be a portion of a pore. The pore may be contained on a surface (e.g., a filter and/or membrane).

For any of the structural and functional characteristics described herein, methods of determining these characteristics are known in the art.

### Methods of Treatment

In some aspects, provided are methods of treating a HPV-associated disease in an individual, the method comprising administering an effective amount of a composition comprising PBMCs to the individual, wherein the PBMCs comprise at least one HPV antigen delivered intracellularly. In some embodiments, the method further comprises administering an effective amount of one or more immune checkpoint inhibitors to the individual.

In some aspects, provided are methods of treating a HPV-associated disease in an individual, the method comprising administering an effective amount of a composition comprising PBMCs to the individual, wherein the PBMCs comprise at least one HPV antigen delivered intracellularly, and administering an effective amount of an antagonist of CTLA-4 and/or an antagonist of PD-1/PD-L1 to the individual.

In some aspects, provided are methods of treating a HPV-associated disease in an individual, the method comprising administering an effective amount of a composition comprising PBMCs to the individual, wherein the effective amount is about 0.5×10⁴ cells/kg to about 5.0×10⁹ cells/kg, and wherein the PBMCs comprise at least one HPV antigen delivered intracellularly.

In some aspects, provided are methods of treating a HPV-associated disease in an individual, the method comprising administering an effective amount of a composition comprising PBMCs to the individual, wherein the effective amount is about 0.5×10⁶ cells/kg to about 5.0×10⁶ cells/kg, and wherein the PBMCs comprise at least one HPV antigen delivered intracellularly.

In some embodiments, the HPV-associated disease is a HPV-associated cancer. In some embodiments, the HPV-associated cancer is cervical cancer, perianal cancer, anogenital cancer, oral cancer, salivary cancer, oropharyngeal cancer, vaginal cancer, vulvar cancer, penile cancer, skin cancer, esophageal cancer, or head and neck cancer. In some embodiments, the HPV-associated disease is a HPV-associated infectious disease.

In some embodiments, the effective amount of PBMCs is about any one of 0.5×10⁴, 1.0×10⁴, 0.5×10⁵, 1.0×10⁵, 0.5×10⁶, 1.0×10⁶, 0.5×10⁷, 1.0×10⁷, 0.5×10⁸, 1.0×10⁸, 0.5×10⁹, and 1.0×10⁹ cells/kg. In some embodiments, the effective amount is any one of about 0.5×10⁴ to about 1.0×10⁴, about 1.0×10⁵ to about 0.5×10⁵, about 0.5×10⁵ to about 1.0×10⁵, about 1.0×10⁵ to about 0.5×10⁶, about 0.5×10⁶ to about 1.0×10⁶, about 1.0×10⁶ to about 0.5×10⁷, about 0.5×10⁷ to about 1.0×10⁷, about 1.0×10⁷ to about 0.5×10⁸, about 0.5×10⁸ to about 1.0×10⁸, about 1.0×10⁸ to about 0.5×10⁹, or about 0.5×10⁹ to about 1.0×10⁹ cells/kg. In some embodiments, provided are methods of treating a HPV-associated cancer in an individual, the method comprising administering an effective amount of a composition comprising PBMCs to the individual wherein the effective amount is about 0.5×10⁶ to about 5×10⁶ PBMCs/kg, and wherein the PBMCs comprise at least one HPV antigen delivered intracellularly.

In some embodiments, the method further comprises administering an effective amount of one or more immune checkpoint inhibitors. Exemplary immune checkpoint inhibitor is an antagonist of, without limitation, PD-1, PD-L1, CTLA-4, LAG3, TIM-3, TIGIT, VISTA, TIM1, B7-H4 (VTCN1) or BTLA. In some embodiments, the immune checkpoint inhibitor is an antagonist of one or more of PD-1, PD-L1, CTLA-4, LAG3, TIM-3, TIGIT, VISTA, TIM1, B7-H4 (VTCN1) or BTLA. In some embodiments, the immune checkpoint inhibitor is one or more of: an antibody that binds to PD-1, an antibody that binds PD-L1, an antibody that binds CTLA-4, an antibody that binds LAG3, an antibody that binds TIM-3, an antibody that binds TIGIT, an antibody that binds VISTA, an antibody that binds TIM-1, an antibody that binds B7-H4, or an antibody that binds BTLA. In further embodiments, the antibody can be a full length antibody or any variants, for example but not limited to, an antibody fragment, a single chain variable fragment (ScFv), or a fragment antigen-binding (Fab). In further embodiments, the antibody can be bispecific, trispecific or multispecific. In some embodiments, the immune checkpoint inhibitor is one or more chemical compounds that binds to and/or inhibits one or more of PD-1, PD-L1, CTLA-4, LAG3, TIM-3, TIGIT, VISTA, TIM1, B7-H4 (VTCN1) or BTLA. In some embodiments, the immune checkpoint inhibitor is one or more peptides that binds to and/or inhibits one or more of PD-1, PD-L1, CTLA-4, LAG3, TIM-3, TIGIT, VISTA, TIM1, B7-H4 (VTCN1) or BTLA. In some embodiments, the immune checkpoint inhibitor is targeted to PD-1. In some embodiments, the immune checkpoint inhibitor is targeted to PD-L1. In some embodiments, the immune checkpoint inhibitor is targeted to CTLA-4.

In some embodiments, provided are methods of treating a HPV-associated cancer in an individual, the method comprising administering an effective amount of a composition comprising PBMCs to the individual, wherein the PBMCs comprise at least one HPV antigen delivered intracellularly, and administering an effective amount of one or more immune checkpoint inhibitors. In some embodiments, provided are methods of treating a HPV-associated cancer in an individual, the method comprising administering an effective amount of a composition comprising PBMCs to the individual wherein the effective amount is about 0.5×10⁶ to about 5×10⁶ PBMCs, and wherein the PBMCs comprise at least one HPV antigen delivered intracellularly, and administering an effective amount of one or more immune checkpoint inhibitors. In some embodiments, the immune checkpoint inhibitor is an antagonist of CTLA-4. In some embodiments, the immune checkpoint inhibitor is an antagonist of PD-1. In some embodiments, the immune checkpoint inhibitor is an antagonist of PD-L1. In some embodiments, the one or more immune checkpoint inhibitors comprise an antagonist of CTLA-4, an antagonist of PD-1, and/or an antagonist of PD-L1. In some embodiments, the immune checkpoint inhibitor is an antibody that binds CTLA-4. In some embodiments, the antagonist of PD-1 is an antibody that binds PD-1. In some embodiments, the antagonist of PD-L1 is an antibody that binds PD-L1. In some embodiments, the one or more immune checkpoint inhibitors comprise an antibody that binds CTLA-4, an antibody that binds PD-1, and/or an antibody that binds PD-L1.

In some embodiments, provided are methods of treating a HPV-associated cancer in an individual, the method comprising administering an effective amount of a composition comprising PBMCs to the individual, wherein the PBMCs comprise at least one HPV antigen delivered intracellularly, and administering an effective amount of: an antagonist of CTLA-4, an antagonist of PD-1, and/or an antagonist of PD-L1. In some embodiments, provided are methods of treating a HPV-associated cancer in an individual, the method comprising administering an effective amount of a composition comprising PBMCs to the individual, wherein the PBMCs comprise at least one HPV antigen delivered intracellularly, and administering an effective amount of: an antibody that binds CTLA-4, an antibody that binds PD-1, and/or an antibody that binds PD-L1. In some embodiments, the antibody that binds PD-1 is nivolumab. In some embodiments, the antibody that binds PD-1 is pembrolizumab. In some embodiments, the antibody that binds PD-L1 is atezolizumab. In some embodiments, the antibody that binds CTLA-4 is ipilimumab. In some embodiments, an antibody that binds CTLA-4 is administered to the individual. In some embodiments, an antibody that binds PD-L1 is administered to the individual. In some embodiments, an antibody that binds PD-1 is administered to the individual.

In some aspects, provided are methods for stimulating an immune response to a HPV antigen in an individual, the method comprising administering an effective amount of a composition comprising PBMCs to an individual, wherein the PBMCs comprise at least one HPV antigen; wherein the at least one HPV antigen is delivered to the PBMC intracellularly. In some embodiments, the PBMCs further comprise an adjuvant. In some embodiments, the method comprises administering an effective amount of any of the compositions described herein. In some embodiments, the individual has cancer.

In some aspects, provided are methods for reducing tumor growth in an individual, the method comprising administering an effective amount of a composition comprising PBMCs to an individual, wherein the PBMCs comprise at least one HPV antigen; wherein the at least one HPV antigen is delivered to the PBMCs intracellularly. In some embodiments, the PBMCs further comprise an adjuvant. In some embodiments, the method comprises administering an effective amount of any of the compositions described herein. In some embodiments, the individual has cancer.

In some aspects, provided are methods for vaccinating an individual in need thereof, the method comprising administering an effective amount of a composition comprising PBMCs to an individual, wherein the PBMCs comprise at least one HPV antigen; wherein the at least one HPV antigen is delivered to the PBMCs intracellularly. In some embodiments, the PBMCs further comprises an adjuvant. In some embodiments, the method comprises administering an effective amount of any of the compositions described herein. In some embodiments, the individual has cancer.

In some aspects, provided are methods for treating cancer in an individual, the method comprising administering an effective amount of a composition comprising PBMCs to an individual, wherein the PBMCs comprise at least one HPV antigen; wherein the at least one HPV antigen is delivered to the PBMCs intracellularly. In some embodiments, the PBMCs further comprises an adjuvant. In some embodiments, the method comprises administering an effective amount of any of the compositions described herein.

In some aspects, there is provided a method for stimulating an immune response to a HPV antigen in an individual, comprising: a) passing a cell suspension comprising input PBMCs through a cell-deforming constriction, wherein a diameter of the constriction is a function of a diameter of the input PBMCs in the suspension, thereby causing perturbations of the input PBMCs enough for a HPV antigen or the HPV antigen and an adjuvant to pass through to form perturbed input PBMCs; b) incubating the perturbed PBMCs with the HPV antigen or the HPV antigen and the adjuvant for a sufficient time to allow the HPV antigen or the HPV antigen and the adjuvant to enter the perturbed input PBMCs; thereby generating PBMCs comprising the HPV antigen or the HPV antigen and the adjuvant; and c) administering an effective amount of the PBMCs comprising the HPV antigen or the HPV antigen and the adjuvant to the individual.

In some aspects, there is provided a method for reducing tumor growth in an individual, comprising: a) passing a cell suspension comprising input PBMCs through a cell-deforming constriction, wherein a diameter of the constriction is a function of a diameter of the input PBMCs in the suspension, thereby causing perturbations of the input PBMCs enough for a HPV antigen or the HPV antigen and an adjuvant to pass through to form perturbed input PBMCs; b) incubating the perturbed PBMCs with the HPV antigen or the HPV antigen and the adjuvant for a sufficient time to allow the HPV antigen or the HPV antigen and the adjuvant to enter the perturbed input PBMCs; thereby generating PBMCs comprising the HPV antigen or the HPV antigen and the adjuvant; and c) administering an effective amount of the PBMCs comprising the HPV antigen or the HPV antigen and the adjuvant to the individual.

In some aspects, there is provided a method for vaccinating an individual in need thereof, comprising: a) passing a cell suspension comprising input PBMCs through a cell-deforming constriction, wherein a diameter of the constriction is a function of a diameter of the input PBMCs in the suspension, thereby causing perturbations of the input PBMCs enough for a HPV antigen or the HPV antigen and an adjuvant to pass through to form perturbed input PBMCs; b) incubating the perturbed PBMCs with the HPV antigen or the HPV antigen and the adjuvant for a sufficient time to allow the HPV antigen or the HPV antigen and the adjuvant to enter the perturbed input PBMCs; thereby generating PBMCs comprising the HPV antigen or the HPV antigen and the adjuvant; and c) administering an effective amount of the PBMCs comprising the HPV antigen or the HPV antigen and the adjuvant to the individual.

In some aspects, there is provided a method for treating cancer in an individual, comprising: a) passing a cell suspension comprising input PBMCs through a cell-deforming constriction, wherein a diameter of the constriction is a function of a diameter of the input PBMCs in the suspension, thereby causing perturbations of the input PBMCs enough for a HPV antigen or the HPV antigen and an adjuvant to pass through to form perturbed input PBMCs; b) incubating the perturbed PBMCs with the HPV antigen or the HPV antigen and the adjuvant for a sufficient time to allow the HPV antigen or the HPV antigen and the adjuvant to enter the perturbed input PBMCs; thereby generating PBMCs comprising the HPV antigen or the HPV antigen and the adjuvant; and c) administering an effective amount of the PBMCs comprising the HPV antigen or the HPV antigen and the adjuvant to the individual.

In some embodiments according to any of the methods, uses or compositions described herein, the methods comprises: a) passing a cell suspension comprising input PBMCs through a cell-deforming constriction, wherein a diameter of the constriction is a function of a diameter of the input PBMCs in the suspension, thereby causing perturbations of the input PBMCs large enough for a HPV antigen to pass through to form perturbed input PBMCs; b) incubating the perturbed input PBMCs with the HPV antigen for a sufficient time to allow the HPV antigen to enter the perturbed input PBMCs; thereby generating modified PBMCs comprising the HPV antigen; and c) administering an effective amount of the modified PBMCs comprising the HPV antigen to the individual.

In some embodiments, there is provided a composition for stimulating an immune response to HPV protein in an individual, wherein the composition comprises an effective amount of any one of the compositions comprising PBMCs comprising at least one HPV antigen as described herein. In some embodiments, there is provided a composition for reducing tumor growth, wherein the composition comprises an effective amount of any one of the compositions comprising PBMCs comprising at least one HPV antigen described herein. In some embodiments, the individual has cancer. In some embodiments, there is provided a composition for treating cancer in an individual, wherein the composition comprises an effective amount of any one of the compositions comprising PBMCs comprising at least one HPV antigen described herein. In some embodiments, the cancer is cervical cancer, perianal cancer, anogenital cancer, oral cancer, salivary cancer, oropharyngeal cancer, vaginal cancer, vulvar cancer, penile cancer, skin cancer, head and neck cancer or esophageal cancer.

In some embodiments, there is provided a composition for stimulating an immune response to HPV protein in an individual, wherein the composition comprises an effective amount of any one of the compositions comprising PBMCs comprising at least one HPV antigen described herein. In some embodiments, there is provided a composition for reducing tumor growth, wherein the composition comprises an effective amount of any one of the compositions comprising PBMCs comprising at least one HPV antigen described herein. In some embodiments, the individual has cancer. In some embodiments, there is provided a composition for treating cancer in an individual, wherein the composition comprises an effective amount of any one of the compositions comprising PBMCs comprising at least one HPV antigen described herein.

In some embodiments, there is provided the use of a composition comprising an effective amount of PBMCs in the manufacture of a medicament for stimulating an immune response to a HPV antigen, wherein the composition comprises an effective amount of any one of the compositions comprising PBMCs comprising at least one HPV antigen described herein. In some embodiments, there is provided the use of a composition comprising an effective amount of PBMCs in the manufacture of a medicament for reducing tumor growth in an individual, wherein the composition comprises an effective amount of any one of the compositions comprising PBMCs comprising at least one HPV antigen described herein. In some embodiments, the individual has cancer. In some embodiments, there is provided the use of a composition comprising an effective amount of PBMCs in the manufacture of a medicament for treating cancer in an individual, wherein the composition comprises an effective amount any one of the compositions comprising PBMCs comprising at least one HPV antigen described herein.

In some embodiments, there is provided the use of a composition comprising an effective amount of PBMCs in the manufacture of a medicament for stimulating an immune response to HPV antigen protein, wherein the composition comprises an effective amount of any one of the compositions comprising PBMCs comprising at least one HPV antigen described herein. In some embodiments, there is provided the use of a composition comprising an effective amount of PBMCs in the manufacture of a medicament for reducing tumor growth in an individual, wherein the composition comprises an effective amount of any one of the compositions comprising PBMCs comprising at least one HPV antigen described herein. In some embodiments, the individual has cancer. In some embodiments, there is provided the use of a composition comprising an effective amount of PBMCs in the manufacture of a medicament for treating cancer in an individual, wherein the composition comprises an effective amount of any one of the compositions comprising PBMCs comprising at least one HPV antigen described herein.

In some embodiments according to the methods, uses or compositions described herein, the individual has cancer. In some embodiments, the cancer is cervical cancer, perianal cancer, anogenital cancer, oral cancer, salivary cancer, oropharyngeal cancer, vaginal cancer, vulvar cancer, penile cancer, skin cancer, head and neck cancer or esophageal cancer. In some embodiments, the cancer is a cancer associated with HPV. In some embodiments, the cancer is a localized cancer. In some embodiments, the cancer is a localized cancer. In some embodiments, the cancer is a locally advanced cancer. In some embodiments, the cancer is a metastatic cancer. In some embodiments, the cancer is a solid tumor. In some embodiments, the cancer is a liquid tumor.

In some embodiments, the cancer is not amenable to curative treatment with surgery, radiation, and/or chemoradiation therapy. In some embodiments, the cancer has progressed after prior systemic chemotherapeutic treatment with a platinum-based regimen in the adjuvant or recurrent setting. In some embodiments, the cancer has progressed after prior chemotherapeutic treatment with one or more of: cisplatin, paclitaxel, carboplatin and/or SFU. In some embodiments, the cancer has progressed after prior immune checkpoint inhibitor treatment. In some embodiments, the cancer has progressed after prior treatment with one or more of: pembrolizumab, bevacizumab and/or pembrolizumab. In some embodiments, the cancer is platinum experienced. In some embodiments, the cancer is platinum resistant. In some embodiments, the individual has a progressive disease while receiving or after the completion of the most recent prior treatment. In some embodiments, the cancer is cervical cancer. In some embodiments, the cancer is recurrent or metastatic cervical cancer.

In some embodiments, the cancer is not amenable to curative treatment with surgery, radiation, and/or chemoradiation therapy. In some embodiments, the cancer has progressed following at least 1 (such as 1, 2, 3, 4, 5, or more) prior platinum-based chemotherapy in the primary, adjuvant or recurrent setting and been offered checkpoint immunotherapy. In some embodiments, the cancer has progressed after prior chemotherapeutic treatment with one or more of: cisplatin, paclitaxel, carboplatin and/or SFU. In some embodiments, the cancer has progressed after prior immune checkpoint inhibitor treatment. In some embodiments, the cancer has progressed after prior treatment with one or more of: pembrolizumab, bevacizumab and/or pembrolizumab. In some embodiments, the cancer is platinum experienced. In some embodiments, the cancer is platinum resistant. In some embodiments, the individual relapsed after platinum-containing definitive chemoradiation or after adjuvant chemoradiation and a platinum re-challenge at time of relapse is not seen as beneficial. In some embodiments, the cancer is head and neck cancer. In some embodiments, the cancer is recurrent or metastatic head and neck cancer.

In some embodiments, the width of the constriction is about 10% to about 99% of the mean diameter of the PBMCs. In some embodiments, the width of the constriction is any one of about 10% to about 90%, about 10% to about 80%, about 10% to about 70%, about 20% to about 60%, about 40% to about 60%, about 30% to about 45%, about 50% to about 99%, about 50% to about 90%, about 50% to about 80%, about 50% to about 70%, about 60% to about 90%, about 60% to about 80%, or about 60% to about 70% of the mean diameter of the input PBMCs having the smallest diameter within the population of PBMCs. In some embodiments, the width of the constriction about 3 μm to about 5 μm, about 3 μm to about 3.5 μm, about 3.5 μm to about 4 μm, about 4 μm to about 4.5 μm, about 3.2 μm to about 3.8 μm, about 3.8 μm to about 4.3 μm, about 4.2 μm to about 6 μm, or about 4.2 μm to about 4.8 μm. In some embodiments, the width of the constriction is about 4.5 μm. In some embodiments, the width of the constriction is about or less than any one of 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, 10.5 μm, 11 μm, 11.5 μm, 12 μm, 12.5 μm, 13 μm, 13.5 μm, 14 μm, 14.5 μm or 15 μm. In some embodiments, the cell suspension comprising the input PBMCs are passed through multiple constrictions wherein the multiple constrictions are arranged in series and/or in parallel.

In some embodiments, the input PBMCs are autologous to the individual. In some embodiments, the input PBMCs are allogeneic to the individual. In some embodiments, the modified PBMCs comprising the HPV antigen are autologous to the individual. In some embodiments, the modified PBMCs comprising the HPV antigen are allogeneic to the individual.

In some embodiments according to any one of the methods, uses or compositions described herein, the PBMCs are incubated with the adjuvant for a sufficient time for the PBMCs to condition. In some embodiments, the PBMCs are incubated with the adjuvant for about 1 to about 24 hours for the PBMCs to condition. In some embodiments, the PBMCs are incubated with the adjuvant for about 2 to about 10 hours for the PBMCs to condition. In some embodiments, the PBMCs are incubated with the adjuvant for about 3 to about 6 hours for the PBMCs to condition. In some embodiments, the PBMCs are incubated with the adjuvant for any one of about 1 hour, 2 hours, 3 hours, 3.5 hours, 4 hours, 4.5 hours, 5 hours, 5.5 hours, 6 hours, 8 hours, 12 hours, 16 hours, 20 hours, or 24 hours for the PBMCs to condition. In some embodiments, the PBMCs are incubated with the adjuvant for about 4 hours for the PBMCs to condition. In some embodiments, the PBMCs are conditioned before introducing the HPV antigen or the nucleic acid encoding the HPV antigen into the PBMCs. In some embodiments, the PBMCs are conditioned after introducing the HPV antigen or the nucleic acid encoding the HPV antigen into the PBMCs. In some embodiments, the adjuvant used for conditioning is a CpG oligodeoxynucleotide (ODN), LPS, IFN-α, IFN-β, IFN-γ, alpha-Galactosyl Ceramide, STING agonists, cyclic dinucleotides (CDN), RIG-I agonists, polyinosinic-polycytidylic (poly I:C), R837, R848, a TLR3 agonist, a TLR4 agonist or a TLR9 agonist. In some embodiments, the adjuvant is a CpG oligodeoxynucleotide (ODN). In some embodiments, the adjuvant is CpG 7909.

In some embodiments, wherein the PBMCs comprise B cells, one or more co-stimulatory molecules is upregulated in the B cells of the conditioned PBMCs compared to the B cells of the unconditioned PBMCs. In some embodiments, one or more co-stimulatory molecules is upregulated in the B cells of the conditioned plurality of PBMCs compared to the B cells of the unconditioned plurality of PBMCs. In some embodiments, the co-stimulatory molecule is CD80 and/or CD86. In some embodiments, the conditioned plurality of PBMCs has increased expression of one or more of IFN-γ, IL-6, MCP-1, MIP-1P, IP-10, or TNF-α compared to an unconditioned plurality of PBMCs. In some embodiments, the expression of one or more of IFN-γ, IL-6, MCP-1, MIP-1P, IP-10, or TNF-α is increased by more than about 1.2-fold, 1.5-fold, 1.8-fold, 2-fold, 3-fold, 4-fold, 5-fold, 8-fold, or more than 10-fold compared to an unconditioned plurality of PBMCs

In some embodiments, the PBMCs are human cells. In some embodiments, the PBMCs are human cells with a haplotype of HLA-A*02, HLA-A*01, HLA-A*03, HLA-A*24, HLA-A*11, HLA-A*26, HLA-A*32, HLA-A*31, HLA-A*68, HLA-A*29, HLA-A*23, HLA-B*07, HLA-B*44, HLA-B*08, HLA-B*35, HLA-B*15, HLA-B*40, HLA-B*27, HLA-B*18, HLA-B*51, HLA-B*14, HLA-B*13, HLA-B*57, HLA-B*38, HLA-C*07, HLA-C*04, HLA-C*03, HLA-C*06, HLA-C*05, HLA-C*12, HLA-C*02, HLA-C*01, HLA-C*08, or HLA-C*16. In some embodiments, the plurality of PBMCs comprises two or more of T cell, B cell, NK cell, monocytes, dendritic cells or NK-T cells. In some embodiments, the PBMCs are one or more of T cells, B cells, NK cells, monocytes, dendritic cells and/or NK-T cells.

In some embodiments, the plurality of PBMCs are further modified to increase expression of one or more of co-stimulatory molecules. In some embodiments, the co-stimulatory molecule is B7-H2 (ICOSL), B7-1 (CD80), B7-2 (CD86), CD70, LIGHT, HVEM, CD40, 4-1BBL, OX40L, TL1A, GITRL, CD30L, TIM4, SLAM, CD48, CD58, CD155, or CD112. In some embodiments, the plurality of PBMCs are further modified to increase expression of one or more cytokines. In some embodiments, the cytokine is IL-10, IL-15, IL-12, IL-2, IFN-α, IFN-γ, or IL 21.

In some embodiments, the HPV antigen is a pool of multiple polypeptides that elicit a response against the same and or different HPV antigens. In some embodiments, the HPV antigen is a polypeptide comprising one or more antigenic HPV epitope and one or more heterologous peptide sequences. In some embodiments, the HPV antigen complexes with other antigens or with an adjuvant. In some embodiments, the HPV antigen is capable of being processed into an MHC class I-restricted peptide. In some embodiments, the HPV antigen is capable of being processed into an MHC class II-restricted peptide.

In some embodiments, the method comprises multiple administrations of the PBMCs comprising the at least one HPV antigen. In some embodiments, the method comprises about 3 to about 9 administrations of the PBMCs comprising the HPV antigen. In some embodiments, the method comprises about any one of 1, 2, 3, 4, 5, 6, 7, 8, 9, 10, 11, 12, 13, 14 or 15 administrations of the PBMCs comprising the HPV antigen. In some embodiments, the method comprises continuous administrations of the PBMCs comprising the HPV antigen as needed. In some embodiments, the time interval between two successive administrations of the PBMCs comprising the HPV antigen is between about 1 day and about 30 days. In some embodiments, the time interval between two successive administrations of PBMCs comprising the HPV antigen is about 21 days. In some embodiments, the time the time interval between two successive administrations of the PBMCs comprising the HPV antigen is about any one of 1, 2, 3, 4, 5, 6, 7, 8, 10, 12, 14, 16, 20, 25, 30, 35, 40, 45, 50, 55, 60, 65, 70, 75, 80, 85, 90, 95, 100, or 150 days. In some embodiments, the time interval between the first two successive administrations of the PBMCs comprising the HPV antigen is 1 day or 2 days. In some embodiments, the time interval between the first two successive administrations of the PBMCs comprising the HPV antigen is 1 day or 2 days, wherein the method comprises more than 2 administration of the PBMCs comprising the HPV antigen (such as but not limited to 3, 4, 5, 6, 7, 8, 9, 10, 11, 12, 13, 14, 15 or more administrations). In some embodiments, the PBMCs comprising the HPV antigen are administered intravenously, intratumorally, orally and/or subcutaneously. In some embodiments, the PBMCs comprising the HPV antigen are administered intravenously.

In some embodiments, the composition further comprises an adjuvant. In some embodiments, the adjuvant is a CpG oligodeoxynucleotide (ODN), LPS, IFN-α, IFN-γ, alpha-Galactosyl Ceramide, STING agonists, cyclic dinucleotides (CDN), RIG-I agonists, polyinosinic-polycytidylic acid, R837, R848, a TLR3 agonist, a TLR4 agonist or a TLR9 agonist. In some embodiments, the adjuvant is a CpG oligodeoxynucleotide. In some embodiments, the adjuvant is poly I:C.

In some embodiments, the individual is positive for expression of HLA-A*02, HLA-A*01, HLA-A*03, HLA-A*24, HLA-A*11, HLA-A*26, HLA-A*32, HLA-A*31, HLA-A*68, HLA-A*29, HLA-A*23, HLA-B*07, HLA-B*44, HLA-B*08, HLA-B*35, HLA-B*15, HLA-B*40, HLA-B*27, HLA-B*18, HLA-B*51, HLA-B*14, HLA-B*13, HLA-B*57, HLA-B*38, HLA-C*07, HLA-C*04, HLA-C*03, HLA-C*06, HLA-C*05, HLA-C*12, HLA-C*02, HLA-C*01, HLA-C*08, or HLA-C*16.

Immune checkpoints are regulators of the immune system and keep immune responses in check. Immune checkpoint inhibitors can be employed to facilitate the enhancement of immune response. In some embodiments, the composition comprising the PBMCs comprising the HPV antigen is administered in combination with administration of an immune checkpoint inhibitor. In some embodiments, the composition comprising the PBMCs comprising HPV antigen and the immune checkpoint inhibitor are administered simultaneously. In some embodiments, the composition comprising the PBMCs comprising the HPV antigen and the immune checkpoint inhibitor are administered sequentially. In some embodiments, the immune checkpoint inhibitor and/or the PBMCs comprising the HPV antigen are administered intravenously, intratumorally, orally and/or subcutaneously. In some embodiments, the PBMCs comprising the HPV antigen are administered intravenously. In some embodiments, the immune checkpoint inhibitor is administered intravenously, intratumorally, orally and/or subcutaneously.

In some embodiments, the composition comprising the PBMCs comprising the HPV antigen is administered prior to administration of the immune checkpoint inhibitor. In some embodiments, the composition comprising the PBMCs comprising the HPV antigen is administered following administration of the immune checkpoint inhibitor. For example, the composition comprising the PBMCs comprising the HPV antigen is administered from about 1 hour to about 1 week prior to administration of the immune checkpoint inhibitor. For example, in some embodiments, the composition comprising the PBMCs comprising the HPV antigen is administered about 1 hour, about 2 hours, about 3 hours, about 4 hours, about 6 hours, about 8 hours, about 10 hours, about 12 hours, about 14 hours, about 16 hours, about 18 hours, about 20 hours, about 24 hours, about 30 hours, about 36 hours, about 42 hours, about 48 hours, about 60 hours, about 3 days, about 4 days, about 5 days, about 6 days, or about 7 days prior to administration of the immune checkpoint inhibitor. In some embodiments, the composition comprising the PBMCs comprising the HPV antigen is administered from between about 1 hour and about 2 hours, from between about 2 hours and about 3 hours, from between about 3 hours and about 4 hours, from between about 4 hours and about 6 hours, from between about 6 hours and about 8 hours, from between about 8 hours and about 10 hours, from between about 10 hours and about 12 hours, from between about 12 hours and about 14 hours, from between about 14 hours and about 16 hours, from between about 16 hours and about 18 hours, from between about 18 hours and about 20 hours, from between about 20 hours and about 24 hours, from between about 24 hours and about 30 hours, from between about 30 hours and about 36 hours, from between about 36 hours and about 42 hours, from between about 42 hours and about 48 hours, from between about 48 hours and about 60 hours, from between about 60 hours and about 3 days, from between about 3 days and about 4 days, from between about 4 days and about 5 days, from between about 5 days and about 6 days, from between about 6 days and about 7 days prior to administration of the immune checkpoint inhibitor.

In some embodiments, the composition comprising the PBMCs comprising the HPV antigen is administered about 7 days, about 10 days, about 14 days, about 18 days, about 21 days, about 24 days, about 28 days, about 30 days, about 35 days, about 40 days, about 45 days, or about 50 days prior to administration of the immune checkpoint inhibitor. In some embodiments, the composition comprising the PBMCs comprising the HPV antigen is administered from between about 7 days to about 10 days, from between about 10 days and about 14 days, from between about 14 days and about 18 days, from between about 18 days and about 21 days, from between about 21 days and about 24 days, from between about 24 days and about 28 days, from between about 28 days and about 30 days, from between about 30 days and about 35 days, from between about 35 days and about 40 days, from between about 40 days and about 45 days, or from between about 45 days and about 50 days prior to administration of the immune checkpoint inhibitor.

In some embodiments, the composition comprising the PBMCs comprising the HPV antigen is administered following administration of the immune checkpoint inhibitor. For example, the composition comprising the PBMCs comprising the HPV antigen is administered from about 1 hour to about 1 week following administration of the immune checkpoint inhibitor. For example, in some embodiments, the composition comprising the PBMCs comprising the HPV antigen is administered about 1 hour, about 2 hours, about 3 hours, about 4 hours, about 6 hours, about 8 hours, about 10 hours, about 12 hours, about 14 hours, about 16 hours, about 18 hours, about 20 hours, about 24 hours, about 30 hours, about 36 hours, about 42 hours, about 48 hours, about 60 hours, about 3 days, about 4 days, about 5 days, about 6 days, or about 7 days following administration of the immune checkpoint inhibitor. In some embodiments, the composition comprising the PBMCs comprising the HPV antigen is administered from between about 1 hour and about 2 hours, from between about 2 hours and about 3 hours, from between about 3 hours and about 4 hours, from between about 4 hours and about 6 hours, from between about 6 hours and about 8 hours, from between about 8 hours and about 10 hours, from between about 10 hours and about 12 hours, from between about 12 hours and about 14 hours, from between about 14 hours and about 16 hours, from between about 16 hours and about 18 hours, from between about 18 hours and about 20 hours, from between about 20 hours and about 24 hours, from between about 24 hours and about 30 hours, from between about 30 hours and about 36 hours, from between about 36 hours and about 42 hours, from between about 42 hours and about 48 hours, from between about 48 hours and about 60 hours, from between about 60 hours and about 3 days, from between about 3 days and about 4 days, from between about 4 days and about 5 days, from between about 5 days and about 6 days, from between about 6 days and about 7 days following administration of the immune checkpoint inhibitor.

In some embodiments, the composition comprising the PBMCs comprising the HPV antigen is administered about 7 days, about 10 days, about 14 days, about 18 days, about 21 days, about 24 days, about 28 days, about 30 days, about 35 days, about 40 days, about 45 days, or about 50 days following administration of the immune checkpoint inhibitor. In some embodiments, the composition comprising the PBMCs comprising the HPV antigen is administered from between about 7 days to about 10 days, from between about 10 days and about 14 days, from between about 14 days and about 18 days, from between about 18 days and about 21 days, from between about 21 days and about 24 days, from between about 24 days and about 28 days, from between about 28 days and about 30 days, from between about 30 days and about 35 days, from between about 35 days and about 40 days, from between about 40 days and about 45 days, or from between about 45 days and about 50 days following administration of the immune checkpoint inhibitor.

In some embodiments, the method comprises multiple administration of the composition comprising the PBMCs comprising the HPV antigen and/or multiple administration of the immune checkpoint inhibitor. For example, in some embodiments, the method comprises two administrations, three administrations, four administrations, five administrations, six administrations, seven administrations, eight administrations, nine administrations, ten administrations, eleven administrations, twelve administrations, thirteen administrations, fourteen administrations, or fifteen administrations of the composition comprising the PBMCs comprising the HPV antigen and/or the immune checkpoint inhibitor. For example, in some embodiments, the method comprises less than five administrations, less than ten administrations, less than fifteen administrations, less than twenty administrations, less than twenty-five administrations, less than thirty administrations, less than fifty administrations, less than seventy-five administrations, less than one hundred, or less than two hundred administrations of the composition comprising the PBMCs comprising the HPV antigen and/or the immune checkpoint inhibitor.

Exemplary immune checkpoint inhibitor is targeted to, without limitation, PD-1, PD-L1, CTLA-4, LAG3, TIM-3, TIGIT, VISTA, TIM1, B7-H4 (VTCN1) or BTLA. In some embodiments, the immune checkpoint inhibitor is targeted to one or more of PD-1, PD-L1, CTLA-4, LAG3, TIM-3, TIGIT, VISTA, TIM1, B7-H4 (VTCN1) or BTLA. In some embodiments, the immune checkpoint inhibitor is one or more of: an antibody that binds to PD-1, an antibody that binds PD-L1, an antibody that binds CTLA-4, an antibody that binds LAG3, or an antibody that binds TIM-3, an antibody that binds TIGIT, an antibody that binds VISTA, an antibody that binds TIM-1, an antibody that binds B7-H4, or an antibody that binds BTLA. In further embodiments, the antibody can be a full length antibody or any variants, for example but not limited to, an antibody fragment, a single chain variable fragment (ScFv), or a fragment antigen-binding (Fab). In further embodiments, the antibody can be bispecific, trispecific or multispecific. In some embodiments, the immune checkpoint inhibitor is one or more chemical compounds that binds to and/or inhibits one or more of PD-1, PD-L1, CTLA-4, LAG3, TIM-3, TIGIT, VISTA, TIM1, B7-H4 (VTCN1) or BTLA. In some embodiments, the immune checkpoint inhibitor is one or more peptides that binds to and/or inhibits one or more of PD-1, PD-L1, CTLA-4, LAG3, TIM-3, TIGIT, VISTA, TIM1, B7-H4 (VTCN1) or BTLA. In some embodiments, the immune checkpoint inhibitor is targeted to PD-1. In some embodiments, the immune checkpoint inhibitor is targeted to PD-L1.

In some embodiments, there is provided a plurality of PBMCs comprising at least one HPV antigen for use in a method of stimulating an immune response in an individual according to any one of the methods described herein.

In some embodiments according to any one of the methods of treating HPV-associated caner described herein, the treatment includes any or all of killing cancer cells, inhibiting growth of cancer cells, inhibiting replication of cancer cells, lessening of overall tumor burden and ameliorating one or more symptoms associated with the disease. In some embodiments, the method of treating HPV associated cancer is more efficacious in a patient with a lower tumor burden than in a patient with a higher tumor burden. In some embodiments, the method of treating HPV associated cancer results in a higher rate in one or more of: killing cancer cells, inhibiting growth of cancer cells, inhibiting replication of cancer cells, lessening of overall tumor burden and ameliorating one or more symptoms associated with the disease in a patient with a lower tumor burden as compared to that in a patient with a higher tumor burden.

### Compositions of PBMCs Comprising HPV Antigens

In some embodiments of the methods of treatment described herein, the PBMCs comprise a HPV antigen and an adjuvant delivered intracellularly. In some embodiments, the PBMCs comprising the at least one HPV antigen are conditioned. In some embodiments, the PBMCs comprising the at least one HPV antigen are conditioned by a process comprising incubating the PBMCs with an adjuvant for about 2 hours to about 10 hours, about 3 hours to about 6 hours, or about 4 hours at about 37° C. for the PBMCs to condition.

In some embodiments, the methods of treatment comprises administering an effective amount of PBMCs comprising at least one HPV antigen, wherein the PBMCs comprising the at least one HPV antigen t are prepared by: a) passing a cell suspension comprising input PBMCs through a cell-deforming constriction, wherein a diameter of the constriction is a function of a diameter of the input PBMCs in the suspension, thereby causing perturbations of the input PBMCs large enough for the at least one HPV antigen to pass through to form perturbed input PBMCs; and b) incubating the perturbed input PBMCs with the at least one HPV antigen for a sufficient time to allow the at least one HPV antigen to enter the perturbed input PBMCs; thereby generating modified PBMCs comprising the at least one HPV antigen. In some embodiments, the HPV antigen comprises the amino acid sequence of any one of SEQ ID NOs:1-4 and 18-25. In some embodiments, the HPV antigen comprises an amino acid sequence with at least 90% identity to any one of SEQ ID NOs:1-4 and 18-25.

In some embodiments, the method comprises administering an effective amount of PBMCs comprising a HPV antigen and an adjuvant, wherein the PBMCs comprising the HPV antigen and the adjuvant are prepared by: a) passing a cell suspension comprising input PBMCs through a cell-deforming constriction, wherein a diameter of the constriction is a function of a diameter of the input PBMCs in the suspension, thereby causing perturbations of the input PBMCs large enough for the HPV antigen and the adjuvant to pass through to form perturbed input PBMCs; and b) incubating the perturbed input PBMCs with the HPV antigen and the adjuvant for a sufficient time to allow the HPV antigen and the adjuvant to enter the perturbed input PBMCs; thereby generating modified PBMCs comprising the HPV antigen and the adjuvant. In some embodiments, the HPV antigen comprises the amino acid sequence of any one of SEQ ID NOs:1-4 and 18-25. In some embodiments, the HPV antigen comprises an amino acid sequence with at least 90% identity to any one of SEQ ID NOs:1-4 and 18-25.

In some aspects, there is provided a composition of PBMCs comprising at least one HPV antigen, wherein the PBMCs comprising the at least one HPV antigen are prepared by: a) passing a cell suspension comprising input PBMCs through a cell-deforming constriction, wherein a diameter of the constriction is a function of a diameter of the input PBMCs in the suspension, thereby causing perturbations of the input PBMCs large enough for the at least one HPV antigen to pass through to form perturbed input PBMCs; and b) incubating the perturbed input PBMCs with the at least one HPV antigen for a sufficient time to allow the at least one HPV antigen to enter the perturbed input PBMCs; thereby generating modified PBMCs comprising the at least one HPV antigen. In some embodiments, the HPV antigen comprises the amino acid sequence of any one of SEQ ID NOs: 1-4 and 18-25. In some embodiments, the HPV antigen comprises an amino acid sequence with at least 90% identity to any one of SEQ ID NOs:1-4 and 18-25.

In some embodiments, the width of the constriction is about 10% to about 99% of the mean diameter of the input PBMCs. In some embodiments, the width of the constriction is any one of about 10% to about 90%, about 10% to about 80%, about 10% to about 70%, about 20% to about 60%, about 40% to about 60%, about 30% to about 45%, about 50% to about 99%, about 50% to about 90%, about 50% to about 80%, about 50% to about 70%, about 60% to about 90%, about 60% to about 80%, or about 60% to about 70% of the mean diameter of the input PBMCs having the smallest diameter within the population of PBMCs. In some embodiments, the width of the constriction is any one of about 10% to about 90%, about 10% to about 80%, about 10% to about 70%, about 20% to about 60%, about 40% to about 60%, about 30% to about 45%, about 50% to about 99%, about 50% to about 90%, about 50% to about 80%, about 50% to about 70%, about 60% to about 90%, about 60% to about 80%, or about 60% to about 70% of the mean diameter of the input PBMCs. In some embodiments, the width of the constriction about 3 μm to about 5 μm, about 3 μm to about 3.5 μm, about 3.5 μm to about 4 μm, about 4 μm to about 4.5 μm, about 3.2 μm to about 3.8 μm, about 3.8 μm to about 4.3 μm, about 4.2 μm to about 6 μm, or about 4.2 μm to about 4.8 μm. In some embodiments, the width of the constriction is about 4.5 μm. In some embodiments, the width of the constriction is about or less than any one of 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, 10.5 μm, 11 μm, 11.5 μm, 12 μm, 12.5 μm, 13 μm, 13.5 μm, 14 μm, 14.5 μm or 15 μm. In some embodiments, the cell suspension comprising the input PMBCs are passed through multiple constrictions wherein the multiple constrictions are arranged in series and/or in parallel. In some embodiments, the cell suspension comprising the input PBMCs are passed through multiple constrictions wherein the multiple constrictions are arranged in series and/or in parallel.

In some embodiments, the HPV antigen is a pool of multiple polypeptides that elicit a response against the same and or different HPV antigens. In some embodiments, the HPV antigen is a polypeptide comprising one or more antigenic HPV epitope and one or more heterologous peptide sequences. In some embodiments, the HPV antigen complexes with other antigens or with an adjuvant. In some embodiments, the HPV antigen is capable of being processed into an MHC class I-restricted peptide. In some embodiments, the HPV antigen is capable of being processed into an MHC class II-restricted peptide.

In some embodiments, the composition further comprises an adjuvant. In some embodiments, the adjuvant is a CpG oligodeoxynucleotide (ODN), LPS, IFN-α, IFN-γ, alpha-Galactosyl Ceramide, STING agonists, cyclic dinucleotides (CDN), RIG-I agonists, polyinosinic-polycytidylic acid (poly I:C), R837, R848, a TLR3 agonist, a TLR4 agonist or a TLR9 agonist. In some embodiments, the adjuvant is polyinosinic-polycytidylic acid (poly I:C).

### Doses and Regimens

In some embodiments, provided are methods of treating a HPV-associated disease in an individual, the method comprising administering an effective amount of a composition comprising PBMCs to the individual wherein the effective amount is about 0.5×10⁶ to about 5×10⁶ cells/kg, and wherein the PBMCs comprise at least one HPV antigen delivered intracellularly. In some embodiments, the method further comprises administering an effective amount of one or more immune checkpoint inhibitors.

In some embodiments according to any one of the methods described herein the effective amount of PBMCs comprising the at least one HPV antigen 0.5×10⁶ to about 5×10⁶ cells/kg. In some embodiments, the effective amount of PBMCs comprising the at least one HPV antigen is about any one of 0.5×10⁴, 1.0×10⁴, 0.5×10⁵, 1.0×10⁵, 0.5×10⁶, 1.0×10⁶, 0.5×10⁷, 1.0×10⁷, 0.5×10⁸, 1.0×10⁸, 0.5×10⁹, and 1.0×10⁹ cells/kg. In some embodiments, the effective amount is any one of about 0.5×10⁴ to about 1.0×10⁴, about 1.0×10⁵ to about 0.5×10⁵, about 0.5×10⁵ to about 1.0×10⁵, about 1.0×10⁵ to about 0.5×10⁶, about 0.5×10⁶ to about 1.0×10⁶, about 1.0×10⁶ to about 0.5×10⁷, about 0.5×10⁷ to about 1.0×10⁷, about 1.0×10⁷ to about 0.5×10⁸, about 0.5×10⁸ to about 1.0×10⁸, about 1.0×10⁸ to about 0.5×10⁹, or about 0.5×10⁹ to about 1.0×10⁹ cells/kg. In some embodiments, provided are methods of treating a HPV-associated cancer in an individual, the method comprising administering an effective amount of a composition comprising PBMCs to the individual wherein the effective amount is about 0.5×10⁶ to about 5×10⁶ cells/kg, and wherein the PBMCs comprise at least one HPV antigen delivered intracellularly.

In some embodiments, wherein the method further comprises administering an effective amount of immune checkpoint inhibitor, wherein the immune checkpoint inhibitor is targeted to CTLA-4. In some embodiments, the immune checkpoint inhibitor is an antagonist of CTLA-4. In some embodiments, the immune checkpoint inhibitor is an antibody that binds CTLA-4. In some embodiments, the immune checkpoint inhibitor is ipilimumab. In some embodiments, the effective amount of ipilimumab is about 0.1 mg/kg to about 30 mg/kg. In some embodiments, the effective amount of ipilimumab is any one of about 1 mg/kg to about 3 mg/kg. In some embodiments, the effective amount of ipilimumab is about any one of 0.1, 0.2, 0.5, 1.0, 1.2, 1.4, 1.6, 1.8, 2.0, 2.2, 2.4, 2.6, 2.8, 3, 4, 5, 6, 7, 8, 9, 10, 12, 14, 16, 18, 20, 25, or 30 mg/kg. In some embodiments, the effective amount of ipilimumab is about any one of 0.1 to 0.2, 0.2 to 0.5, 0.5 to 1.0, 1.0 to 1.2, 1.2 to 1.4, 1.4 to 1.6, 1.6 to 1.8, 1.8 to 2.0, 2.0 to 2.2, 2.2 to 2.4, 2.4 to 2.6, 2.6 to 2.8, 2.8 to 3, 3 to 4, 4 to 5, 5 to 6, 6 to 7, 7 to 8, 8 to 9, 9 to 10, 10 to 12, 12 to 14, 14 to 16, 16 to 18, 18 to 20, 20 to 25, or 25 to 30 mg/kg.

In some embodiments, wherein the method further comprises administering an effective amount of immune checkpoint inhibitor, wherein the immune checkpoint inhibitor is targeted to PD-1. In some embodiments, the immune checkpoint inhibitor is an antagonist of PD-1. In some embodiments, the immune checkpoint inhibitor is an antibody that binds PD-1. In some embodiments, the immune checkpoint inhibitor is nivolumab. In some embodiments, the effective amount of nivolumab is about 30 mg to about 1000 mg. In some embodiments, the effective amount of nivolumab is any one of about 300 mg to about 400 mg. In some embodiments, the effective amount of nivolumab is about 360 mg. In some embodiments, the effective amount of nivolumab is about any one of 30, 50, 100, 150, 200, 250, 300, 320, 340, 360, 380, 400, 450, 500, 550, 600, 700, 800, 900 or 1000 mg. In some embodiments, the effective amount of ipilimumab is about any one of 30 to 50, 50 to 100, 100 to 150, 150 to 200, 200 to 250, 250 to 300, 300 to 320, 320 to 340, 340 to 360, 360 to 380, 380 to 400, 400 to 450, 500 to 550, 550 to 600, 600 to 700, 700 to 800, 800 to 900, 900 to 1000 mg.

In some embodiments, wherein the method further comprises administering an effective amount of immune checkpoint inhibitor, wherein the immune checkpoint inhibitor is targeted to PD-L1. In some embodiments, the immune checkpoint inhibitor is an antagonist of PD-L1. In some embodiments, the immune checkpoint inhibitor is an antibody that binds PD-L1. In some embodiments, the immune checkpoint inhibitor is atezolizumab. In some embodiments, the effective amount of atezolizumab is about 100 mg to about 2500 mg. In some embodiments, the effective amount of atezolizumab is about 900 mg to about 1500 mg. In some embodiments, the effective amount of atezolizumab is any one of about 1200 mg. In some embodiments, the effective amount of atezolizumab is about any one of 100, 200, 300, 400, 500, 600, 700, 800, 900, 1000, 1100, 1150, 1200, 1250, 1300, 1400, 1500, 1600, 1800, 2000, 2200 or 2500 mg. In some embodiments, the effective amount of atezolizumab is about any one of 100 to 200, 200 to 300, 300 to 400, 400 to 500, 500 to 600, 600 to 700, 700 to 800, 800 to 900, 900 to 1000, 1000 to 1100, 1100 to 1200, 1200 to 1300, 1300 to 1400, 1400 to 1500, 1500 to 1600, 1600 to 1800, 1800 to 2000, 2000 to 2200, 2200 to 2500 mg.

In some embodiments, the method of treatment comprises multiple (such as any of 2, 3, 4, 5, 6, 7, 8, 9, 10 or more) cycles of administering the PBMCs as described herein to the individual. For example, in some embodiments, there is provided a method of vaccinating an individual against an antigen by administering an PBMCs comprising at least one HPV antigen, generated by passing input PBMCs through a constriction to form perturbed input PBMCs such that the at least one HPV antigen enters the PBMCs, to the individual 2, 3, 4, 5, 6, 7, 8, 9, 10 or more times. In some embodiments, the duration of time between any two consecutive administrations of the modified PBMCs is at least about 1 day (such at least about any of 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 1 year, or longer, including any ranges between these values).

In some embodiments according to any one of the methods described herein, the composition comprising the PBMCs is administered in any one of a 1-, 2-, 3-, 4-, 5-, 6-, 7-, 8-, 9-, or 10-week cycle. In some embodiments, the composition comprising the PBMCs is administered on day 1 in any one of a 1-, 2-, 3-, 4-, 5-, 6-, 7-, 8-, 9-, or 10-week cycle. In some embodiments, the composition comprising the PBMCs is administered in a 3-week cycle. In some embodiments, the composition comprising the PBMCs is administered in a 6-week cycle. In some embodiments, the composition comprising the PBMCs is administered on one or more of day 1, 2, 3, 4, 5, 6, 7, 8, 9, 10, 11, 12, 13, 14, 15, 16, 17, 18, 19, or 20 in the treatment cycle. In some embodiments, the composition comprising the PBMCs is administered on day 1 of a treatment cycle. In some embodiments, the composition comprising the PBMCs is administered on day 2 of a treatment cycle. In some embodiments, the composition comprising the PBMCs is administered on day 1 and day 2 of a treatment cycle. In some embodiments, the composition comprising the PBMCs is administered on day 1 and day 3 of a treatment cycle. In some embodiments, the composition comprising the PBMCs is administered on day 8 of a treatment cycle. In some embodiments, the composition comprising the PBMCs is administered on day 1 of a three-week cycle. In some embodiments, the composition comprising the PBMCs is further administered on day 2 of a three-week cycle. In some embodiments, the composition comprising the PBMCs is administered in 3-week cycles until the PBMC composition supply is exhausted, or for one year. In some embodiments, the composition comprising PBMCs is administered to the individual for at least about three months, six months, nine months, one year, or two years.

In some embodiments, any one of about 0.5×10⁴, 1.0×10⁴, 0.5×10⁵, 1.0×10⁵, 0.5×10⁶, 1.0×10⁶, 0.5×10⁷, 1.0×10⁷, 0.5×10⁸, 1.0×10⁸, 0.5×10⁹, and 1.0×10⁹ cells/kg of PBMCs are administered on day 1 of each three-week cycle. In some embodiments, about 0.5×10⁶ cells/kg to about 5×10⁶ cells/kg are administered on day 1 of each three-week cycle. In some embodiments, about 0.5×10⁶ cells/kg, about 2.5×10⁶ cells/kg, or about 5.0×10⁶ cells/kg are administered on day 1 of each three-week cycle. In some embodiments, any one of about 0.5×10⁴ 1.0×10⁴, 0.5×10⁵, 1.0×10⁵, 0.5×10⁶, 1.0×10⁶, 0.5×10⁷, 1.0×10⁷, 0.5×10⁸, 1.0×10⁸, 0.5×10⁹, and 1.0×10⁹ cells/kg are administered on day 2 of each three-week cycle. In some embodiments, any one of about 0.5×10⁴, 1.0×10⁴, 0.5×10⁵, 1.0×10⁵, 0.5×10⁶, 1.0×10⁶, 0.5×10⁷, 1.0×10⁷, 0.5×10⁸, 1.0×10⁸, 0.5×10⁹, and 1.0×10⁹ cells/kg are administered on day 1 and on day 2 of a first three-week cycle and any one of about 0.5×10⁴, 1.0×10⁴, 0.5×10⁵, 1.0×10⁵, 0.5×10⁶, 1.0×10⁶, 0.5×10⁷, 1.0×10⁷, 0.5×10⁸, 1.0×10⁸, 0.5×10⁹, and 1.0×10⁹ cells/kg are administered on day 1 of subsequent three-week cycles. In some embodiments, about 0.5×10⁶ cells/kg, about 2.5×10⁶ cells/kg, or about 5.0×10⁶ cells/kg are administered on days 1 and 2 of a first three-week cycle and about 0.5×10⁶ cells/kg, about 2.5×10⁶ cells/kg, or about 5.0×10⁶ cells/kg are administered on day 1 of subsequent three-week cycles. In some embodiments, about 0.5×10⁶ cells/kg to about 5×10⁶ cells/kg are administered on day 2 of each three-week cycle. In some embodiments, about 0.5×10⁶ cells/kg, about 2.5×10⁶ cells/kg, or about 5.0×10⁶ cells/kg are administered on day 2 of each three-week cycle. In some embodiments, 0.5×10⁶ cells/kg are administered on day 1 of each three-week cycle. In some embodiments, 0.5×10⁶ cells/kg are administered on day 1 of each three-week cycle, and 0.5×10⁶ cells/kg are administered on day 2 of each three-week cycle. In some embodiments, 0.5×10⁶ cells/kg are administered on day 1 of each three-week cycle, and 0.5×10⁶ cells/kg are administered on day 3 of each three-week cycle. In some embodiments, 2.5×10⁶ cells/kg are administered on day 1 of each three-week cycle. In some embodiments, 2.5×10⁶ cells/kg are administered on day 1 of each three-week cycle, and 2.5×10⁶ cells/kg are administered on day 2 of each three-week cycle. In some embodiments, 2.5×10⁶ cells/kg are administered on day 1 of each three-week cycle, and 2.5×10⁶ cells/kg are administered on day 3 of each three-week cycle. In some embodiments, 2.5×10⁶ cells/kg are administered on day 1 of each three-week cycle. In some embodiments, 5×10⁶ cells/kg are administered on day 1 of each three-week cycle, and 5×10⁶ cells/kg are administered on day 2 of each three-week cycle. In some embodiments, 5×10⁶ cells/kg are administered on day 1 of each three-week cycle, and 5×10⁶ cells/kg are administered on day 3 of each three-week cycle.

In some embodiments, wherein the method further comprises administering an effective amount of one or more immune checkpoint inhibitors, the immune checkpoint inhibitors are targeted to CTLA-4. PD-1 and/or PD-L1. In some embodiments, the antibody that binds CTLA-4 and/or the antibody that binds PD-1 and/or the antibody that binds PD-L1 is administered 1, 2, 3, 4, 5, 6 or more times per cycle. In some embodiments, the antibody that binds CTLA-4 and/or the antibody that binds PD-1 and/or the antibody that binds PD-L1 is administered once per three-week cycle. In some embodiments, the antibody that binds CTLA-4 is administered once per three-week cycle. In some embodiments, the antibody that binds PD-1 is administered once per three-week cycle. In some embodiments, the antibody that binds PD-L1 is administered once per three-week cycle. In some embodiments, the antibody that binds CTLA-4 is administered once per two three-week cycles. In some embodiments, the antibody that binds PD-1 is administered once per two three-week cycles. In some embodiments, the antibody that binds PD-L1 is administered once per two three-week cycles.

In some embodiments according to any one of the methods described herein, the immune checkpoint inhibitor is administered in any one of a 1-, 2-, 3-, 4-, 5-, 6-, 7-, 8-, 9-, or 10-week cycle. In some embodiments, the immune checkpoint inhibitor is administered on day 1 in any one of a 1-, 2-, 3-, 4-, 5-, 6-, 7-, 8-, 9-, or 10-week cycle. In some embodiments, the immune checkpoint inhibitor is administered on one or more times on day 1, 2, 3, 4, 5, 6, 7, 8, 9, 10, 11, 12, 13, 14, 15, 16, 17, 18, 19, or 20 in the treatment cycle.

In some embodiments, the immune checkpoint inhibitor is an antibody binding CTLA-4, wherein the antibody that binds CTLA-4 is administered on day 1 of each three-week cycle. In some embodiments, the antibody that binds CTLA-4 is administered for a maximum of four doses. In some embodiments, the effective amount of the antibody that binds CTLA-4 is about 1 mg/kg to about 3 mg/kg. In some embodiments, the antibody that binds CTLA-4 is ipilimumab. In some embodiments, the ipilimumab is administered at a dose of about 1 mg/kg to about 3 mg/kg. In some embodiments, the antibody that binds CTLA-4 is ipilimumab, wherein the ipilimumab is administered on day 1 of every three-week cycle at a dose of about 3 mg/kg.

In some embodiments, the immune checkpoint inhibitor is an antibody binding PD-1, wherein the antibody that binds PD-1 is administered on day 8 of the first three-week cycle and on day 1 of each subsequent three-week cycle. In some embodiments, the antibody that binds PD-1 is nivolumab. In some embodiments, the nivolumab is administered at a dose of about 360 mg. In some embodiments, the antibody that binds PD-1 is nivolumab, wherein the nivolumab is administered on day 8 of the first three-week cycle and day 1 of each subsequent cycle at a dose of about 360 mg.

In some embodiments, the one of more immune checkpoint inhibitors comprise an antibody binding CTLA-4 and an antibody binding PD-1, wherein the antibody that binds CTLA-4 is administered on day 1 of every alternate three-week cycle (i.e. day 1 of every 6-week cycle, or day 1 of the first three-week cycle of two three-week cycles) and wherein the antibody that binds PD-1 is administered on day 8 of the first three-week cycle and on day 1 of each subsequent three-week cycle. In some embodiments, the antibody that binds CTLA-4 is ipilimumab and the antibody that binds PD-1 is nivolumab. In some embodiments, the ipilimumab is administered at a dose of about 1 mg/kg. In some embodiments, the nivolumab is administered at a dose of about 360 mg. In some embodiments, the antibody that binds CTLA-4 is ipilimumab, wherein the ipilimumab is administered on day 1 of every alternate three-week cycle (i.e. day 1 of every 6-week cycle, or day 1 of the first three-week cycle of two three-week cycles) at a dose of about 1 mg/kg and the antibody that binds PD-1 is nivolumab, wherein the nivolumab is administered on day 8 of the first three-week cycle and day 1 of each subsequent cycle at a dose of about 360 mg.

In some embodiments, the immune checkpoint inhibitor is an antibody binding PD-L1, wherein the antibody that binds PD-L1 is administered on day 8 of the first three-week cycle and on day 1 of each subsequent three-week cycle. In some embodiments, the antibody that binds PD-L1 is atezolizumab. In some embodiments, the atezolizumab is administered at a dose of about 1200 mg. In some embodiments, the antibody that binds PD-1 is atezolizumab, wherein the atezolizumab is administered on day 8 of the first three-week cycle and day 1 of each subsequent cycle at a dose of about 1200 mg.

### Methods of Generating Compositions of PBMCs Comprising HPV Antigen

In some embodiments, provided are methods for generating a composition comprising PBMCs comprising at least one HPV antigen, wherein the at least one HPV antigen is delivered to the PBMCs intracellularly. For example, methods for generating a composition for use in the methods of treatment as described herein. In some embodiments, provided are methods for generating a composition comprising PBMCs comprising a HPV antigen and an adjuvant, wherein the HPV antigen and the adjuvant is delivered to the PBMCs intracellularly.

In some embodiments, the PBMCs comprising the at least one HPV antigen are prepared by a process comprising: a) passing a cell suspension comprising a population of input PBMCs through a cell-deforming constriction, wherein a diameter of the constriction is a function of a diameter of the input PBMCs in the suspension, thereby causing perturbations of the input PBMCs large enough for the at least one HPV antigen to pass through to form perturbed input PBMCs; and b) incubating the population of perturbed input PBMCs with the at least one HPV antigen and the adjuvant for a sufficient time to allow the antigen to enter the perturbed input PBMCs, thereby generating the modified PBMCs comprising the at least one HPV antigen.

In some embodiments, the HPV antigen comprises a peptide derived from HPV E6. In some embodiments, the HPV antigen comprises a peptide derived from HPV E7. In some embodiments, the HPV antigen comprises a peptide derived from HPV E6

In some embodiments, the width of the constriction is about 10% to about 99% of the mean diameter of the input PBMCs. In some embodiments, the width of the constriction is any one of about 10% to about 90%, about 10% to about 80%, about 10% to about 70%, about 20% to about 60%, about 40% to about 60%, about 30% to about 45%, about 50% to about 99%, about 50% to about 90%, about 50% to about 80%, about 50% to about 70%, about 60% to about 90%, about 60% to about 80%, or about 60% to about 70% of the mean diameter of the input PBMCs. In some embodiments, the width of the constriction is about 3 μm to about 15 μm. In some embodiments, the width of the constriction is about 3 μm to about 10 μm. In some embodiments, the width of the constriction is about 3 μm to about 6 μm. In some embodiments, the width of the constriction is about 4.2 μm to about 6 μm. In some embodiments, the width of the constriction is about 4.2 μm to about 4.8 μm. In some embodiments, the width of the constriction is about 3 μm to about 5 μm. In some embodiments, the width of the constriction is about 3 μm to about 3.5 μm. In some embodiments, the width of the constriction is about 3.5 μm to about 4 μm. In some embodiments, the width of the constriction is about 4 μm to about 4.5 μm. In some embodiments, the width of the constriction is about 3.2 μm to about 3.8 μm. In some embodiments, the width of the constriction is about 3.8 μm to about 4.3 μm. In some embodiments, the width of the constriction is about or less than any one of 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, 10.5 μm, 11 μm, 11.5 μm, 12 μm, 12.5 μm, 13 μm, 13.5 μm, 14 μm, 14.5 μm or 15 μm. In some embodiments, the width of the constriction is about or less than any one of 3.0 μm, 3.1 μm, 3.2 μm, 3.3 μm, 3.4 μm, 3.5 μm, 3.6 μm, 3.7 μm, 3.8 μm, 3.9 μm, 4.0 μm, 4.1 μm, 4.2 μm, 4.3 μm, 4.4 μm, 4.5 μm, 4.6 μm, 4.7 μm, 4.8 μm, 4.9 μm, or 5.0 μm. In some embodiments, the width of the constriction is about 4.5 μm. In some embodiments, the cell suspension comprising the input PBMCs are passed through multiple constrictions wherein the multiple constrictions are arranged in series and/or in parallel.

In some embodiments, the HPV antigen is a pool of multiple polypeptides that elicit a response against the same and or different HPV antigens. In some embodiments, the HPV antigen is a polypeptide comprising one or more antigenic HPV epitope and one or more heterologous peptide sequences. In some embodiments, the HPV antigen is delivered with other antigens or with an adjuvant. In some embodiments, the HPV antigen is a polypeptide comprising an antigenic HPV epitope and one or more heterologous peptide sequences. In some embodiments, the HPV antigen complexes with itself, with other antigens, or with the adjuvant. In some embodiments, the HPV is HPV-16 or HPV-18. In some embodiments, the HPV antigen is comprised of an HLA-A2-specific epitope. In some embodiments, the HPV antigen is a HPV E6 antigen or a HPV E7 antigen. In some embodiments, the antigen comprises a peptide derived from HPV E6 and/or E7. In some embodiments, the antigen comprises an HLA-A2-restricted peptide derived from HPV E6 and/or E7. In some embodiments, the HPV antigen is capable of being processed into an MHC class I-restricted peptide. In some embodiments, the HPV antigen is capable of being processed into an MHC class II-restricted peptide.

In some embodiments, the composition further comprises an adjuvant. In some embodiments, the adjuvant is a CpG oligodeoxynucleotide (ODN), LPS, IFN-α, IFN-β, IFN-γ, alpha-Galactosyl Ceramide, STING agonists, cyclic dinucleotides (CDN), RIG-I agonists, polyinosinic-polycytidylic acid (poly I:C), R837, R848, a TLR3 agonist, a TLR4 agonist or a TLR9 agonist. In some embodiments, the adjuvant is polyinosinic-polycytidylic acid (poly I:C).

### HPV Antigens

In some embodiments according to the methods described herein, the exogenous antigen is a HPV antigen. Papillomaviruses are small nonenveloped DNA viruses with a virion size of ˜55 nm in diameter. More than 100 HPV genotypes are completely characterized, and a higher number is presumed to exist. HPV is a known cause of cervical cancers, as well as some vulvar, vaginal, penile, oropharyngeal, anal, and rectal cancers. Although most HPV infections are asymptomatic and clear spontaneously, persistent infections with one of the oncogenic HPV types can progress to precancer or cancer. Other HPV-associated diseases can include common warts, plantar warts, flat warts, anogenital warts, anal lesions, epidermodysplasia, focal epithelial hyperplasia, mouth papillomas, verrucous cysts, laryngeal papillomatosis, squamous intraepithelial lesions (SILs), cervical intraepithelial neoplasia (CIN), vulvar intraepithelial neoplasia (VIN) and vaginal intraepithelial neoplasia (VAIN). Many of the known HPV types cause benign lesions with a subset being oncogenic. Based on epidemiologic and phylogenetic relationships, HPV types are classified into fifteen “high-risk types” (HPV 16, 18, 31, 33, 35, 39, 45, 51, 52, 56, 58, 59, 68, 73, and 82) and three “probable high-risk types” (HPV 26, 53, and 66), which together are known to manifest as low and high grade cervical changes and cancers, as well as other anogenital cancers such as vulval, vaginal, penile, anal, and perianal cancer, as well as head and neck cancers. Recently, the association of high-risk types HPV 16 and 18 with breast cancer was also described. Eleven HPV types classified as “low-risk types” (HPV 6, 11, 40, 42, 43, 44, 54, 61, 70, 72, and 81) are known to manifest as benign low-grade cervical changes, genital warts and recurrent respiratory papillomatosis. Cutaneous HPV types 5, 8, and 92 are associated with skin cancer. In some HPV-associated cancers, the immune system is depressed and correspondingly, the antitumor response is significantly impaired. See Suresh and Burtness, Am J Hematol Oncol 13(6):20-27 (2017). In some embodiments, the exogenous antigen is a pool of multiple polypeptides that elicit a response against the same and or different antigens. In some embodiments, an antigen in the pool of multiple antigens does not decrease the immune response directed toward other antigens in the pool of multiple antigens. In some embodiments, the HPV antigen is a polypeptide comprising an antigenic HPV epitope and one or more heterologous peptide sequences. In some embodiments, the HPV antigen complexes with itself, with other antigens, or with the adjuvant. In some embodiments, the HPV is HPV-16 or HPV-18. In some embodiments, the HPV antigen is comprised of an HLA-A2-specific epitope. In some embodiments, the HPV antigen is a HPV E6 antigen or a HPV E7 antigen. In some embodiments, the antigen comprises a peptide derived from HPV E6 and/or E7. In some embodiments, the antigen comprises an HLA-A2-restricted peptide derived from HPV E6 and/or E7. In some embodiments, the antigen comprises an HLA-A2-restricted peptide derived from HPV E6 and/or E7, wherein the HLA-A2 restricted peptide comprises the amino acid sequence of any one of SEQ ID NOs: 1-4. In some embodiments, the HLA-A2 restricted peptide comprises the amino acid sequence of SEQ ID NO: 1. In some embodiments, the HLA-A2 restricted peptide comprises the amino acid sequence of SEQ ID NO: 2. In some embodiments, the HLA-A2 restricted peptide comprises the amino acid sequence of SEQ ID NO: 3. In some embodiments, the HLA-A2 restricted peptide comprises the amino acid sequence of SEQ ID NO: 4. In some embodiments, the HLA-A2-restricted peptide comprises the amino acid sequence of any one of SEQ ID NOs:18-25. In some embodiments, the HPV antigen comprises an amino acid sequence with at least 90% similarity to any one of SEQ ID NOs:18-25. In some embodiments, the HPV antigen comprises an amino acid sequence with at least 90% similarity to SEQ ID NO:18. In some embodiments, the HPV antigen comprises an amino acid sequence with at least 90% similarity to SEQ ID NO:19. In some embodiments, the HPV antigen comprises the amino acid sequence of SEQ ID NO:20. In some embodiment, the HPV antigen consists of the amino acid sequence of SEQ ID NO:21. In some embodiments, the HPV antigen comprises the amino acid sequence of SEQ ID NO:22. In some embodiments, the HPV antigen consists of the amino acid sequence of SEQ ID NO:23. In some embodiments, the HPV antigen consists of the amino acid sequence of SEQ ID NO:24. In some embodiments, the HPV antigen consists of the amino acid sequence of SEQ ID NO:25. In some embodiments, the HPV antigen comprises the amino acid sequence of any one of SEQ ID NOs:18-25. In some embodiments, the HPV antigen is a plurality of antigens comprising at least one of the amino acid sequences of any one of SEQ ID NOs:18-25. In some embodiments, the exogenous antigen is a plurality of antigens comprising 2, 3, 4, 5, 6, 7 or 8 of the amino acid sequences of any one of SEQ ID Nos:18-25. In some embodiments, the exogenous antigen is a plurality of antigens comprising an amino acid sequence with at least 90% similarity to SEQ ID NO:19 and an amino acid sequence with at least 90% similarity to SEQ ID NO:23 In some embodiments, the exogenous antigen is a plurality of antigens comprising the amino acid sequence of SEQ ID NO:19 and the amino acid sequence of SEQ ID NO:23. In some embodiments, the plurality of antigens is contained within a pool of non-covalently linked peptides. In some embodiments, the plurality of antigens is contained within a pool of non-covalently linked peptides, wherein each peptide comprises no more than one antigen. In some embodiments, the plurality of antigens is contained within a pool of non-covalently linked peptides, wherein the amino acid sequence of SEQ ID NO:19 and the amino acid sequence of SEQ ID NO:25 are contained within separate peptides

In some embodiments, the HPV antigen is within a pool of multiple polypeptides that elicit a response against the same and or different HPV antigens. In some embodiments, an antigen in the pool of multiple antigens does not decrease the immune response directed toward other antigens in the pool of multiple antigens. In some embodiments, the HPV antigen is a polypeptide comprising an antigenic HPV antigen and one or more heterologous peptide sequences. In some embodiments, the HPV antigen complexes with itself, with other antigens, or with the adjuvant. In some embodiments, the HPV antigen is comprised of an HLA-A2-specific epitope. In some embodiments, the HPV antigen is comprised of an HLA-A11-specific epitope. In some embodiments, HPV antigen is comprised of an HLA-B7-specific epitope. In some embodiments, the HPV antigen is comprised of an HLA-C8-specific epitope. In some embodiments, the HPV antigen comprises part or all of the N-terminal domain of a full-length HPV protein.

In some embodiments according to any one of the methods described herein, the PBMCs comprise a plurality of HPV antigens that comprise a plurality of immunogenic epitopes. In further embodiments, following administration to an individual of the PBMCs comprising the plurality of antigens that comprise the plurality of immunogenic epitopes, none of the plurality of immunogenic epitopes decreases an immune response in the individual to any of the other immunogenic epitopes. In some embodiments, the HPV antigen is a polypeptide and the immunogenic epitope is an immunogenic peptide epitope. In some embodiments, the immunogenic peptide epitope is fused to an N-terminal flanking polypeptide and/or a C-terminal flanking polypeptide. In some embodiments, the HPV antigen is a polypeptide comprising an immunogenic peptide epitope and one or more heterologous peptide sequences. In some embodiments, the HPV antigen is a polypeptide comprising an immunogenic peptide epitope that is flanked on the N-terminus and/or the C-terminus by heterologous peptide sequences. In some embodiments, the flanking heterologous peptide sequences are derived from disease-associated immunogenic peptides. In some embodiments, the flanking heterologous peptide sequences are non-naturally occurring sequence. In some embodiments, the flanking heterologous peptide sequences are derived from an immunogenic synthetic long peptide (SLP). In some embodiments, the HPV antigen is capable of being processed into an MHC class I-restricted peptide and/or an MHC class II-restricted peptide.

### Adjuvants

As used herein, the term “adjuvant” can refer to a substance which either directly or indirectly modulates and/or engenders an immune response. In some embodiments of the invention, an adjuvant is delivered intracellularly to a population of PBMCs to form modified PBMCs comprising the adjuvant. In some instances, the adjuvant is administered in conjunction with PBMCs comprising a HPV antigen to effect enhancement of an immune response to the HPV antigen as compared to HPV antigen alone. In some embodiments, the PBMCs are incubated with the adjuvant before, during, or after the passage of PBMCs through the constrictions, to facilitate conditioning (for example but not limited to maturation) of the PBMCs. Adjuvants can be used to boost elicitation of an immune cell response (e.g. T cell response) to a HPV antigen. Exemplary adjuvants include, without limitation, stimulator of interferon genes (STING) agonists, retinoic acid-inducible gene I (RIG-I) agonists, and agonists for TLR3, TLR4, TLR7, TLR8 and/or TLR9. Exemplary adjuvants include, without limitation, CpG ODN, interferon-α (IFN-α), polyinosinic:polycytidylic acid (polyI:C), imiquimod (R837), resiquimod (R848), or lipopolysaccharide (LPS). In some embodiments, the adjuvant is CpG ODN, LPS, IFN-α, IFN-β, IFN-γ, alpha-Galactosyl Ceramide, STING agonists, cyclic dinucleotides (CDN), RIG-I agonists, polyinosinic:polycytidylic acid (polyI:C), R837, R848, a TLR3 agonist, a TLR4 agonist or a TLR9 agonist. In particular embodiments, the adjuvant is a CpG ODN. In some embodiments, the adjuvant is a CpG ODN. In some embodiments, the CpG ODN is a Class A CpG ODN, a Class B CpG ODN, or a Class C CpG ODN. In some embodiments, the CpG ODN adjuvant comprise of a selection from the group of CpG ODN 1018, CpG ODN 1585, CpG ODN 2216, CpG ODN 2336, CpG ODN 1668, CpG ODN 1826, CPG ODN 2006, CpG ODN 2007, CpG ODN BW006, CpG ODN D-SL01, CpG ODN 2395, CpG ODN M362, CpG ODN D-SL03. In some embodiments, the CpG ODN adjuvant is CpG ODN 1826 (TCCATGACGTTCCTGACGTT (SEQ ID NO:30)) or CpG ODN 2006 (also known as CpG 7909) (TCGTCGTTTTGTCGTTTTGTCGTT (SEQ ID NO:31)) oligonucleotide. In some embodiments, the adjuvant is CpG 7909. In some embodiments, the RIG-I agonist comprises polyinosinic:polycytidylic acid (polyI:C). Multiple adjuvants can also be used in conjunction with HPV antigens to enhance the elicitation of immune response. In some embodiments, the PBMCs comprising the HPV antigen further comprise more than one adjuvant. In some embodiments, the PBMCs comprising the HPV antigen is conditioned by more than one adjuvant. Multiple adjuvants can also be used in conjunction with HPV antigens to enhance the elicitation of immune response. In some embodiments, the PBMCs comprising the HPV antigen further comprise more than one adjuvant. In some embodiments, the PBMCs comprising the HPV antigen further comprise any combination of the adjuvants CpG ODN, LPS, IFN-α, IFN-β,IFN-γ, alpha-Galactosyl Ceramide, STING agonists, cyclic dinucleotides (CDN), RIG-I agonists, polyinosinic:polycytidylic acid (polyI:C), R837, R848, a TLR3 agonist, a TLR4 agonist or a TLR9 agonist. In some embodiments, the PBMCs comprising the HPV antigen are conditioned by any combination of the adjuvants CpG ODN, LPS, IFN-α, IFN-β, IFN-γ, alpha-Galactosyl Ceramide, STING agonists, cyclic dinucleotides (CDN), RIG-I agonists, polyinosinic:polycytidylic acid (polyI:C), R837, R848, a TLR3 agonist, a TLR4 agonist or a TLR9 agonist.

Constituent Cells within the PBMCs

In some embodiments, the methods disclosed herein provide for the administration to an individual in need thereof an effective amount of compositions of PBMCs comprising at least one HPV antigen, wherein the at least one HPV antigen is delivered intracellularly. In some embodiments, the composition of PMBCs is a composition of immune cells. In some embodiments, the composition of PBMCs comprises a plurality of PBMCs. In some embodiments, the PBMCs are one or more of T cells, B cells, NK cells, monocytes, dendritic cells and/or NK-T cells.

In a particular embodiment of the invention, the cells comprising a HPV antigen of the composition are PBMCs. As used herein, PBMCs may be isolated by apheresis such as leukapheresis from whole blood obtained from an individual. Also provided are PBMC compositions reconstituted by mixing different pools of PBMCs from the same individual or different individuals. In other examples, PBMCs may also be reconstituted by mixing different populations of cells into a mixed cell composition with a generated profile. In some embodiments, the populations of cells used for reconstituting PBMCs are mixed populations of cells (such as a mixture of one or more of T cells, B cells, NK cells or monocytes). In some embodiments, the populations of cells used for reconstituting PBMCs are purified populations of cells (such as purified T cells, B cells, NK cells or monocytes). In additional examples, the different populations of cells used in reconstituting a PBMC composition can be isolated from the same individual (e.g. autologous) or isolated from different individuals (e.g. allogenic and/or heterologous).

Therefore, in some embodiments according to the methods described herein, the plurality of PBMCs comprises one or more of T cells, B cells, NK cells, monocytes, dendritic cells or NK-T cells. In some embodiments, the plurality of PBMCs comprises T cells, B cells, NK cells, monocytes, dendritic cells or NK-T cells. In some embodiments, the plurality of PBMCs comprises one or more of CD3+ T cells, CD20+ B cells, CD14+ monocytes, CD56+ NK cells. In some embodiments, the plurality of PBMCs comprises T cells, B cells, NK cells and monocytes, and the ratio of T cells, B cells, NK cells and monocytes to the total number of PBMCs in the plurality of PBMCs is essentially the same as the ratio of T cells, B cells, NK cells and monocytes to the total number of PBMCs in whole blood. In some embodiments, the plurality of PBMCs comprises T cells, B cells, NK cells and monocytes, and the ratio of T cells, B cells, NK cells and monocytes to the total number of PBMCs in the plurality of PBMCs is essentially the same as the ratio of T cells, B cells, NK cells and monocytes to the total number of PBMCs in a leukapheresis product from whole blood. In some embodiments, the plurality of PBMCs comprises T cells, B cells, NK cells and monocytes, and the ratio of T cells, B cells, NK cells and monocytes to the total number of PBMCs in the plurality of PBMCs differs by not more than any one of 1%, 2%, 5%, 10% 15%, 20%, 25%, 30%, 40%, or 50% from the ratio of T cells, B cells, NK cells and monocytes to the total number of PBMCs in whole blood. In some embodiments, the plurality of PBMCs comprises T cells, B cells, NK cells and monocytes, and the ratio of T cells, B cells, NK cells and monocytes to the total number of PBMCs in the plurality of PBMCs differs by not more than any one of 10% from the ratio of T cells, B cells, NK cells and monocytes to the total number of PBMCs in whole blood. In some embodiments, the plurality of PBMCs comprises T cells, B cells, NK cells and monocytes, and the ratio of T cells, B cells, NK cells and monocytes to the total number of PBMCs in the plurality of PBMCs differs by not more than any one of 1%, 2%, 5%, 10% 15%, 20%, 25%, 30%, 40%, or 50% from the ratio of T cells, B cells, NK cells and monocytes to the total number of PBMCs in a leukapheresis product from whole blood. In some embodiments, the plurality of PBMCs comprises T cells, B cells, NK cells and monocytes, and the ratio of T cells, B cells, NK cells and monocytes to the total number of PBMCs in the plurality of PBMCs differs by not more than any one of 10% from the ratio of T cells, B cells, NK cells and monocytes to the total number of PBMCs in a leukapheresis product from whole blood.

In some embodiments according to the methods described herein, about 25% to about 80% of the modified PBMCs are T cells. In some embodiments, about 1.5% to about 30% of the modified PBMCs are B cells. In some embodiments, about 3% to about 35% of the modified PBMCs are NK cells. In some embodiments, about 4% to about 45% of the modified PBMCs are NK cells.

In some embodiments according to the methods described herein, at least about any one of 15%, 20%, 25%, 30%, 35%, 40%, 45%, 50%, 55%, 60%, 65%, 70%, 75%, or 80% of the PBMCs are T cells. In some embodiments, at least about 25% of the PBMCs are T cells. In some embodiments, at least about any one of 0.5%, 1%, 1.5%, 2%, 2.5%, 3%, 4%, 5%, 6%, 7%, 7.5%, 8%, 9%, 10%, 11%, 12%, 13%, 14%, 15%, 16%, 17%, 18%, 19%, 20%, 25%, or 30% of the PBMCs are B cells. In some embodiments, at least about 1.5% of the PBMCs are B cells. In some embodiments, at least about any one of 0.5%, 1%, 1.5%, 2%, 2.5%, 3%, 4%, 5%, 6%, 7%, 7.5%, 8%, 9%, 10%, 11%, 12%, 13%, 14%, 15%, 16%, 17%, 18%, 19%, 20%, 25%, or 30% of the PBMCs are NK cells. In some embodiments, at least about 3% of the PBMCs are NK cells. In some embodiments, at least about any one of 1%, 2%, 3%, 4%, 5%, 6%, 7%, 8%, 9%, 10%, 12%, 14%, 16%, 18%, 20%, 25%, 30%, 35%, 40% or 45% of the PBMCs are monocytes. In some embodiments, at least about 4% of the PBMCs are monocytes. In some embodiments, at least about 25% of the PBMCs are T cells; at least about 1.5% of the PBMCs are B cells; at least about 3% of the PBMCs are NK cells; and at least about 4% of the PBMCs are monocytes.

In some embodiments according to the methods described herein, not more than about any one of 40%, 45%, 50%, 55%, 60%, 65%, 70%, 75%, 80%, 85%, or 90% of the PBMCs are T cells. In some embodiments, not more than about 80% of the PBMCs are T cells. In some embodiments, not more than about any one of 5%, 10%, 12%, 14%, 16%, 18%, 20%, 22%, 25%, 30%, 35%, 40%, or 50% of the PBMCs are B cells. In some embodiments, not more than about 30% of the PBMCs are B cells. In some embodiments, not more than about any one of 10%, 15%, 20%, 25%, 30%, 35%, 40%, 45%, 50% or 60% of the PBMCs are NK cells. In some embodiments, not more than about 35% of the PBMCs are NK cells. In some embodiments, not more than about any one of 5%, 10%, 12%, 14%, 16%, 18%, 20%, 22%, 25%, 30%, 35%, 40%, or 50% of the PBMCs are monocytes. In some embodiments, not more than about 45% of the PBMCs are monocytes. In some embodiments, not more than about 80% of the PBMCs are T cells; not more than about 30% of the PBMCs are B cells; not more than about 20% of the PBMCs are NK cells; and not more than about 45% of the PBMCs are monocytes.

In some embodiments according to the methods described herein, about any one of 20% to 25%, 25% to 30%, 30% to 35%, 35% to 40%, 40% to 45%, 45% to 50%, 50% to 55%, 55% to 60%, 60% to 65%, 65% to 70%, 70% to 75%, or 75% to 80% of the modified PBMCs are T cells. In some embodiments, about 25% to about 80% of the modified PBMCs are T cells. In some embodiments, about any one of 1% to 1.5%, 1.5% to 2.5%, 2.5% to 4%, 4% to 6%, 6% to 8%, 8% to 10%, 10% to 12%, 12% to 14%, 14% to 16%, 16% to 20%, 20% to 25%, or 25% to 30% of the modified PBMCs are B cells. In some embodiments, about 2.5% to about 14% of the modified PBMCs are B cells. In some embodiments, about any one of 1% to 2%, 2% to 3%, 3% to 5%, 5% to 8%, 8% to 10%, 10% to 12%, 12% to 14%, 14% to 16%, 16% to 20%, 20% to 25%, 25% to 30%, 30% to 35%, or 35% to 40% of the modified PBMCs are NK cells. In some embodiments, about 3.0% to about 35% of the modified PBMCs are NK cells. In some embodiments, about any one of 2% to 4%, 4% to 6%, 6% to 8%, 8% to 10%, 10% to 12%, 12% to 14%, 14% to 16%, 16% to 20%, 20% to 25%, 25% to 30%, 30% to 35%, 35% to 40%, or 30% to 45% of the modified PBMCs are monocytes. In some embodiments, about 4% to about 45% of the modified PBMCs are monocytes. In some embodiments, about 25% to about 80% of the modified PBMCs are T cells, about 1.5% to about 30% of the modified PBMCs are B cells, about 3% to about 35% of the modified PBMCs are NK cells, and about 4% to about 45% of the modified PBMCs are monocytes. In some embodiments, about 25% to about 80% of the modified PBMCs are T cells, about 1.5% to about 30% of the modified PBMCs are B cells, about 3% to about 20% of the modified PBMCs are NK cells, and about 4% to about 45% of the modified PBMCs are monocytes. In some embodiments, about 25% to about 70% of the modified PBMCs are T cells, about 2.5% to about 14% of the modified PBMCs are B cells, about 3.5% to about 20% of the modified PBMCs are NK cells, and about 4% to about 25% of the modified PBMCs are monocytes.

As used herein, PBMCs can also be generated after manipulating the composition of a mixed cell population of mononuclear blood cells (such as lymphocytes and monocytes). In some instances, the PBMCs are generated after reducing (such as depleting) certain subpopulations (such as B cells) within a mixed cell population of mononuclear blood cells. The composition in a mixed cell population of mononuclear blood cells in an individual can be manipulated to make the cell population more closely resemble a leukapheresis product from whole blood in the same individual. In other examples, the composition in a mixed cell population of mononuclear blood cells (for example, mouse splenocytes) can also be manipulated to make the cell population more closely resemble human PBMCs isolated from a leukapheresis product from human whole blood.

In some embodiments of the invention, the composition of PMBCs comprising at least one HPV antigen is a population of cells found in PBMCs. In some embodiments, the composition of PMBCs comprising at least one HPV antigen comprises one or more of T cells, B cells, NK cells, monocytes, dendritic cells or NK-T cells. In some embodiments, the composition of PMBCs comprising at least one HPV antigen comprises one or more of CD3+ T cells, CD20+ B cells, CD14+ monocytes, CD56+ NK cells. In some embodiments, the composition of PMBCs comprising at least one HPV antigen comprises at least about any of 70%, 75%, 80%, 85%, 90%, 95%, or 99% T cells. In some embodiments, the composition of PMBCs comprising at least one HPV antigen comprises 100% T cells. In some embodiments, the composition of PMBCs comprising at least one HPV antigen comprises at least about any of 70%, 75%, 80%, 85%, 90%, 95%, or 99% B cells. In some embodiments, the composition of PMBCs comprising at least one HPV antigen comprises 100% B cells. In some embodiments, the composition of PMBCs comprising at least one HPV antigen comprises at least about any of 70%, 75%, 80%, 85%, 90%, 95%, or 99% NK cells. In some embodiments, the composition of PMBCs comprising at least one HPV antigen comprises 100% NK cells. In some embodiments, the composition of PMBCs comprising at least one HPV antigen comprises at least about any of 70%, 75%, 80%, 85%, 90%, 95%, or 99% monocytes. In some embodiments, the composition of PMBCs comprising at least one HPV antigen comprises 100% monocytes. In some embodiments, the composition of PMBCs comprising at least one HPV antigen comprises at least about any of 70%, 75%, 80%, 85%, 90%, 95%, or 99% dendritic cells. In some embodiments, the composition of PMBCs comprising at least one HPV antigen comprises 100% dendritic cells. In some embodiments, the composition of PMBCs comprising at least one HPV antigen comprises at least about any of 70%, 75%, 80%, 85%, 90%, 95%, or 99% NK-T cells. In some embodiments, the composition of PMBCs comprising at least one HPV antigen comprises 100% NK-T cells.

### Manufacturability of PBMCs Comprising the at Least One HPV Antigen

In some embodiments according to any one of the methods or compositions described herein, the viability of PBMCs comprising at least one HPV antigen is at least about any one of: 30%, 40%, 50%, 60%, 70%, 80%, 90%, 95% or 98%. In some embodiments, the viability of PBMCs comprising at least one HPV antigen is at least about 90%.

In some embodiments, the methods of treatment comprises administering an effective amount of PBMCs comprising at least one HPV antigen, wherein the PBMCs comprising the at least one HPV antigens are prepared by: a) passing a cell suspension comprising input PBMCs through a cell-deforming constriction, wherein a diameter of the constriction is a function of a diameter of the input PBMCs in the suspension, thereby causing perturbations of the input PBMCs large enough for the at least one HPV antigen to pass through to form perturbed input PBMCs; and b) incubating the perturbed input PBMCs with the at least one HPV antigen for a sufficient time to allow the at least one HPV antigen to enter the perturbed input PBMCs; thereby generating modified PBMCs comprising the at least one HPV antigen, wherein the viability of PBMCs comprising at least one HPV antigen is at least about any one of: 30%, 40%, 50%, 60%, 70%, 80%, 90%, 95% or 98%.

In some embodiments according to any one of the methods or compositions described herein, the end-to-end processing time for the PBMCs comprising the at least one HPV antigen (such as processing including one or more of: elutriation of patient leukopak, manipulating the composition of the PBMCs, generating and/or conditioning PBMCs comprising the at least one HPV antigens) is about any one of: 4, 5, 6, 7, 8, 9, 10, 11, 12, 13, 14, 15, 16, 17, 18, 19, 20, 21, 22, 23 or 24 hours. In some embodiments, the end-to-end processing time for PBMCs comprising the at least one HPV antigen is about 15 hours.

In some embodiments, the methods of treatment comprises administering an effective amount of PBMCs comprising at least one HPV antigen, wherein the PBMCs comprising the at least one HPV antigen are prepared by: a) passing a cell suspension comprising input PBMCs through a cell-deforming constriction, wherein a diameter of the constriction is a function of a diameter of the input PBMCs in the suspension, thereby causing perturbations of the input PBMCs large enough for the at least one HPV antigen to pass through to form perturbed input PBMCs; and b) incubating the perturbed input PBMCs with the at least one HPV antigen for a sufficient time to allow the at least one HPV antigen to enter the perturbed input PBMCs; thereby generating modified PBMCs comprising the at least one HPV antigen, wherein the end-to-end processing time for the PBMCs comprising the at least one HPV antigen is about any one of: 4, 5, 6, 7, 8, 9, 10, 11, 12, 13, 14, 15, 16, 17, 18, 19, 20, 21, 22, 23 or 24 hours.

In some embodiments, according to any one of the methods or compositions described herein, the modified PBMCs comprising at least one HPV antigen can stimulate at least about 300 pg/mL IFNγ secretion when co-culturing with the HPV antigen-specific responder T cells. In some embodiments, the modified PBMCs comprising at least one HPV antigen can stimulate at least about any one of: 300, 500, 750, 1000, 1500, 2000, 3000, 4000, 5000, 6000, 7000, or 10000 pg/mL IFNγ secretion when co-culturing with the HPV antigen-specific responder T cells. In some embodiments, at least about 90% of batches of the modified PBMCs comprising at least one HPV antigen can stimulate at least about 300 pg/mL IFNγ secretion when co-culturing with the HPV antigen-specific responder T cells. In some embodiments, at least about any one of: 50%, 60%, 70%, 80%, 90%, 95% or 98% of batches of the modified PBMCs comprising at least one HPV antigen can stimulate at least about 300 pg/mL IFNγ secretion when co-culturing with HPV antigen-specific responder T cells. In some embodiments, 100% of batches of the modified PBMCs comprising at least one HPV antigen can stimulate at least about 300 μg/mL IFNγ secretion when co-culturing with HPV antigen-specific responder T cells.

In some embodiments, the methods of treatment comprises administering an effective amount of PBMCs comprising at least one HPV antigen, wherein the PBMCs comprising the at least one HPV antigen are prepared by: a) passing a cell suspension comprising input PBMCs through a cell-deforming constriction, wherein a diameter of the constriction is a function of a diameter of the input PBMCs in the suspension, thereby causing perturbations of the input PBMCs large enough for the at least one HPV antigen to pass through to form perturbed input PBMCs; and b) incubating the perturbed input PBMCs with the at least one HPV antigen for a sufficient time to allow the at least one HPV antigen to enter the perturbed input PBMCs; thereby generating modified PBMCs comprising the at least one HPV antigen, wherein the modified PBMCs comprising at least one HPV antigen can stimulate at least about any one of: 300, 500, 750, 1000, 1500, 2000, 3000, 4000, 5000, 6000, 7000, or 10000 μg/mL IFNγ secretion when co-culturing with HPV antigen-specific responder T cells.

In some embodiments, the methods of treatment comprises administering an effective amount of PBMCs comprising at least one HPV antigen, wherein the PBMCs comprising the at least one HPV antigen are prepared by: a) passing a cell suspension comprising input PBMCs through a cell-deforming constriction, wherein a diameter of the constriction is a function of a diameter of the input PBMCs in the suspension, thereby causing perturbations of the input PBMCs large enough for the at least one HPV antigen to pass through to form perturbed input PBMCs; and b) incubating the perturbed input PBMCs with the at least one HPV antigen for a sufficient time to allow the at least one HPV antigen to enter the perturbed input PBMCs; thereby generating modified PBMCs comprising the at least one HPV antigen, wherein at least two or more batches of the modified PBMCS are prepared, wherein at least about any one of: 50%, 60%, 70%, 80%, 90%, 95% or 98% of batches of the modified PBMCs comprising at least one HPV antigen can stimulate at least about 300 μg/mL IFNγ secretion when co-culturing with HPV antigen-specific responder T cells.

### Further Modifications of PBMCs Comprising the at Least One HPV Antigen

In some embodiments according to any one of the methods described herein, the composition of the PBMCs further comprises an agent that enhances the viability and/or function of the PMBCs as compared to a corresponding composition of PMBCs that does not comprise the agent. In some embodiments, the composition of PBMCs further comprises an agent that enhances the viability and/or function of the PBMCs upon freeze-thaw cycle as compared to a corresponding composition of PBMCs that does not comprise the agent. In some embodiments, the agent is a cryopreservation agent and/or a hypothermic preservation agent. In some embodiments, the cryopreservation agent nor the hypothermic preservation agent cause not more than 10% or 20% of cell death in a composition of PBMCs comprising the agent compared to a corresponding composition of PBMCs that does not comprise the agent before any freeze-thaw cycles. In some embodiments, freeze-thaw cycles of PBMC compositions comprising the cryopreservation agent and/or the hypothermic preservation agent causes 10%, 20%, 30%, 40%, or 50% less loss of viable cells when compared to freeze-thaw cycles of a corresponding PBMC without the cryopreservation agent and the hypothermic preservation agent. In some embodiments, at least about 70%, about 80%, about 90%, or about 95% of the PBMCs are viable after up to 1, 2, 3, 4, 5 freeze-thaw cycles. In some embodiments, at least about 70%, about 80%, or about 90% of the PBMCs are viable after up to 1, 2, 3, 4, 5 freeze-thaw cycles. In some embodiments, the agent is a compound that enhances endocytosis, a stabilizing agent or a co-factor. In some embodiments, the agent is albumin. In some embodiments, the albumin is mouse, bovine, or human albumin. In some embodiments, the agent is one or more of mouse, bovine, or human albumin. In some embodiments, the agent is human albumin. In some embodiments, the agent is one or more of: a divalent metal cation, glucose, ATP, potassium, glycerol, trehalose, D-sucrose, PEG1500, L-arginine, L-glutamine, or EDTA. In some embodiments, the divalent metal cation is one more of Mg2+, Zn2+ or Ca2+. In some embodiments, the agent is one or more of: sodium pyruvate, adenine, trehalose, dextrose, mannose, sucrose, human serum albumin (HSA), DMSO, HEPES, glycerol, glutathione, inosine, dibasic sodium phosphate, monobasic sodium phosphate, sodium metal ions, potassium metal ions, magnesium metal ions, chloride, acetate, gluoconate, sucrose, potassium hydroxide, or sodium hydroxide. In some embodiments, the agent is one or more of: Sodium pyruvate, adenine, Rejuvesol®, trehalose, dextrose, mannose, sucrose, human serum albumin (HSA), PlasmaLyte®, DMSO, Cryostor® CS2, Cryostor® CS5, Cryostor® CS10, Cryostor® CS15, HEPES, glycerol, glutathione, HypoThermosol®.

In some embodiments according to any one of the methods described herein, the composition of PBMCs comprises a plurality of modified PBMCs that are further modified to increase expression of one or more of co-stimulatory molecules. In some embodiments, the co-stimulatory molecule is B7-H2 (ICOSL), B7-1 (CD80), B7-2 (CD86), CD70, LIGHT, HVEM, CD40, 4-1BBL, OX40L, TL1A, GITRL, CD30L, TIM4, SLAM, CD48, CD58, CD155, or CD112. In some embodiments, the plurality of modified PBMCs comprises a nucleic acid that results in increased expression of the one or more co-stimulatory molecules. In some embodiments, the plurality of modified PBMCs comprises an mRNA that results in increased expression of the one or more co-stimulatory molecules. In some embodiments, the co-stimulatory molecule is a Signal 2 effector in stimulating T cell activation.

In some embodiments according to any one of the methods described herein, the modified PBMCs are further modified to increase expression of one or more cytokines. In some embodiments, the cytokine is one or more of IL-2, IL-12, IL-21, or IFNα2. In some embodiments, the plurality of modified PBMCs comprises a nucleic acid that results in increased expression and/or secretion of the one or more cytokines. In some embodiments, the cytokine is a Signal 3 effector in stimulating T cell activation.

In some embodiments according to any one of the methods described herein, at least one cell in the plurality of modified PBMCs is positive for expression of HLA-A2. In some embodiments, the modified PBMCs comprise a further modification to modulate MHC class I expression. In some embodiments, the modified PBMCs comprise a further modification to modulate expression of HLA-A02 MHC class I. In some embodiments, the modified PBMCs comprise a further modification to modulate expression of HLA-A*11 MHC class I. In some embodiments, the modified PBMCs comprise a further modification to modulate expression of HLA-B*07 MHC class I. In some embodiments, the modified PBMCs comprise a further modification to modulate expression of HLA-C*08 MHC class I. Agents that can lead to the upregulation of HLA expression include, but are not limited to, IFNγ, IFNα, IFNβ and radiation.

In some embodiments, the modified PBMCs comprise a further modification to modulate MHC class II expression. In some embodiments, an innate immune response mounted in an individual in response to administration, in an allogeneic context, of the modified PBMCs is reduced compared to an innate immune response mounted in an individual in response to administration, in an allogeneic context, of corresponding modified PBMCs that do not comprise the further modification. In some embodiments, the circulating half-life of the modified PBMCs in an individual to which they were administered is increased compared to the circulating half-life of corresponding modified PBMCs that do not comprise the further modification in an individual to which they were administered. In some embodiments, the circulating half-life of the modified PBMCs in an individual to which they were administered is increased by about any one of 10%, 25%, 50%, 75%, 100%, 2-fold, 3-fold, 4-fold, 5-fold, 10-fold, 25-fold, 50-fold, 100-fold, 200-fold, or 500-fold or more compared to the circulating half-life of corresponding modified PBMCs that do not comprise the further modification in an individual to which they were administered. In some embodiments, the circulating half-life of the modified PBMCs in an individual to which they were administered is essentially the same as the circulating half-life of corresponding modified PBMCs that do not comprise the further modification in an individual to which they were administered.

In some embodiments according to any one of the methods described herein, the process further comprises a step of incubating the composition of PBMCs with an agent that enhances the viability and/or function of the PBMCs compared to corresponding PBMCs prepared without the further incubation step.

In some embodiments, the composition comprises about any one of 0.5×10⁴, 1.0×10⁴, 0.5×10⁵, 1.0×10⁵, 0.5×10⁶, 1.0×10⁶, 0.5×10⁷, 1.0×10⁷, 0.5×10⁸, 1.0×10⁸, 0.5×10⁹, 1.0×10⁹, 0.5×10¹⁰, 1.0×10¹⁰ PBMCs per mL. In some embodiments, the effective amount is any one of about 0.5×10⁴ to about 1.0×10⁴, about 1.0×10⁵ to about 0.5×10⁵, about 0.5×10⁵ to about 1.0×10⁵, about 1.0×10⁵ to about 0.5×10⁶, about 0.5×10⁶ to about 1.0×10⁶, about 1.0×10⁶ to about 0.5×10⁷, about 0.5×10⁷ to about 1.0×10⁷, about 1.0×10⁷ to about 0.5×10⁸, about 0.5×10⁸ to about 1.0×10⁸, about 1.0×10⁸ to about 0.5×10⁹, or about 0.5×10⁹ to about 1.0×10⁹ PBMCs/mL. In some embodiments, the composition comprise about any one of 1×10⁴, 1×10⁵, 1×10⁶, 2×10⁶, 3×10⁶, 4×10⁶, 5×10⁶, 6×10⁶, 7×10⁶, 8×10⁶, 9×10⁶, 1×10⁷, 1×10⁸ PBMCs/mL. In some embodiments, the composition comprises 1×10⁶ PBMCs/mL to about 1×10⁷ PBMCs/mL.

In some embodiments, the composition comprises about 5×10⁴ to about 5×10⁹ PBMCs. In some embodiments, the composition comprises about 5×10⁶ to about 5×10⁷ PBMCs. In some embodiments, the composition comprises about any one of 0.5×10⁴, 1.0×10⁴, 0.5×10⁵, 1.0×10⁵, 0.5×10⁶, 1.0×10⁶, 0.5×10⁷, 1.0×10⁷, 0.5×10⁸, 1.0×10⁸, 0.5×10⁹, 1.0×10⁹ and 5.0×10⁹ PBMCs. In some embodiments, the composition comprises any one of 0.5×10⁴ to about 1.0×10⁴, about 1.0×10⁵ to about 0.5×10⁵, about 0.5×10⁵ to about 1.0×10⁵, about 1.0×10⁵ to about 0.5×10⁶, about 0.5×10⁶ to about 1.0×10⁶, about 1.0×10⁶ to about 0.5×10⁷, about 0.5×10⁷ to about 1.0×10⁷, about 1.0×10⁷ to about 0.5×10⁸, about 0.5×10⁸ to about 1.0×10⁸, about 1.0×10⁸ to about 0.5×10⁹, about 0.5×10⁹ to about 1.0×10⁹, or about 1.0×10⁹ to about 5×10⁹ PBMCs. In some embodiments, the composition comprises about any one of 1×10⁷, 2×10⁷, 3×10⁷, 4×10⁷, 5×10⁷, 6×10⁷, 7×10⁷, 8×10⁷, 9×10⁷, and 1×10⁸ PBMCs. In some embodiments, the composition comprises about 2×10⁷ PBMCs to about 3×10⁷ PBMCs. In some embodiments, the composition comprises about any one of 2.1×10⁷, 2.2×10⁷, 2.3×10⁷, 2.4×10⁷, 2.5×10⁷, 2.6×10⁷, 2.7×10⁷, 2.8×10⁷, 2.9×10⁷, and 3.0×10⁷ PBMCs. In some embodiments, the composition comprises about 2.75×10⁷ PBMCs. In some embodiments, the composition comprises about 2.5×10⁷ PBMCs.

In some embodiments, the composition comprises a cryopreservation medium. In some embodiments, the composition comprises cryopreservation medium at a concentration of about any one of 20%, 25%, 30%, 35%, 40%, 45%, 50%, 55%, 60%, 65%, 70%, 75%, 80%, or 85% (w/w). In some embodiments, the composition comprises cryopreservation medium at a concentration of about any one of 20% to 25%, 25% to 30%, 30% to 35%, 35% to 40%, 40% to 45%, 45% to 50%, 50% to 55%, 55% to 60%, 60% to 65%, 65% to 70%, 70% to 75%, 75% to 80% or 80% to 85% (w/w).

In some embodiments, the composition comprises a hypothermic preservation medium. In some embodiments, the composition comprises hypothermic preservation medium at a percentage of about any one of 10%, 15%, 20%, 25%, 30%, 35%, 40%, 45%, 50%, 55%, 60%, 65%, or 70% (w/w). In some embodiments, the composition comprises hypothermic preservation medium at a percentage of about any one of 10% to 15%, 15% to 20%, 20% to 25%, 25% to 30%, 30% to 35%, 35% to 40%, 40% to 45%, 45% to 50%, 50% to 55%, 55% to 60%, 60% to 65%, or 65% to 70% (w/w).

In some embodiments, the composition comprises human serum albumin at a percentage of about any one of 2%, 3%, 4%, 5%, 8%, or 10% (w/w). In some embodiments, the composition comprises human serum albumin at a percentage of about any one of 2% to 3%, 3% to 5%, 5% to 8%, or 8% to 10% (w/w). In some embodiments, the human serum albumin is added to the formulation as a human serum albumin formulation. In some embodiments, the percentage of the human serum albumin solution in the formulation is about any one of 15%, 20%, 25%, 30%, 35%, 40%, 45%, or 50% (w/w). In some embodiments, the percentage of the human serum albumin solution in the formulation is about any of 10% to 15%, 15% to 20%, 20% to 25%, 25% to 30%, 30% to 35%, 35% to 40%, 40% to 45%, or 45% to 50% (w/w).

In some embodiments, the pH of the formulation is about 5.0 to about 9.5. In some embodiments, the pH of the formulation is about 6.0 to about 8.5. In some embodiments, the pH of the formulation is about 7.4. In some embodiments, the pH of the formulation is any one of about 5, 5.5, 6, 6.5, 7, 7.5, 8, 8.5, 9, 9.5 or 10. In some embodiments, the pH of the formulation is any one of about 7, 7.1, 7.2, 7.3, 7.4, 7.5, 7.6, 7.7, 7.8, 7.9, or 8.0. In some embodiments, the pH of the formulation is any one of about 5 to about 6, about 6 to about 7, about 7 to about 8, about 8 to about 9, or about 9 to about 10. In some embodiments, the pH of the formulation is any one of about 7 to about 7.1, about 7.1 to about 7.2, about 7.2 to about 7.3, about 7.3 to about 7.4, about 7.4 to about 7.5, about 7.5 to about 7.6, about 7.6 to about 7.7, about 7.7 to about 7.8, about 7.8 to about 7.9, or about 7.9 to about 8.0.

In some embodiments, the cryopreservation medium comprises CryoStor® CS10. In some embodiments, the composition comprising PBMCs comprise about 5×10⁶ to about 5×10⁷ PBMCs in CryoStor® CS10.

In some embodiments, the composition comprising PBMCs comprises a) about 5×10⁶ PBMCs to about 5×10⁷ PBMCs; b) cryopreservation medium at a percentage of about 40% to about 60% (w/w); c) hypothermic preservation medium about 25% to about 35% (w/w); and d) human serum albumin about 3% to about 8% (w/w), wherein the pH of the formulation is about pH 6.0 to about pH 8.5.

In some embodiments, the composition comprising PBMCs comprises: a) about 1×10⁶ PBMCs/mL to about 1×10⁷ PBMCs/mL; b) cryopreservation medium at a percentage of about 40% to about 60% (w/w); c) hypothermic preservation medium about 25% to about 35% (w/w); and d) human serum albumin about 3% to about 8% (w/w), wherein the pH of the formulation is about pH 6.0 to about pH 8.5.

In some embodiments, the composition comprising PBMCs comprises: a) about 2.75×10⁷ PBMCs; b) cryopreservation medium at a percentage of about 50% (w/w); c) hypothermic preservation medium at a percentage of about 30% (w/w); and d) human serum albumin at a percentage of about 5% (w/w), wherein the pH of the formulation is about pH 7.4.

In some embodiments, the composition comprising PBMCs comprises: a) about 5×10⁶ PBMCs/mL, b) cryopreservation medium at a percentage of about 50% (w/w), c) hypothermic preservation medium at a percentage of about 30% (w/w), and d) human serum albumin at a percentage of about 5% (w/w), wherein the pH of the formulation is about pH 7.4.

In some embodiments, the composition comprising PBMCs comprises a) about 5×10⁶ PBMCs to about 5×10⁷ PBMCs, b) cryopreservation medium at a percentage of about 65% to about 95% (w/w), c) human serum albumin at a percentage of about 3% to about 8% (w/w), wherein the pH of the formulation is about pH 6.0 to about pH 8.5.

In some embodiments, the composition comprising PBMCs comprises; a) about 1×10⁶ PBMCs/mL to about 1×10⁷ PBMCs/mL, b) cryopreservation medium at a percentage of about 65% to about 95% (w/w), c) human serum albumin at a percentage of about 3% to about 8% (w/w), wherein the pH of the formulation is about pH 6.0 to about pH 8.5.

In some embodiments, the composition comprising PBMCs comprises: a) about 2.75×10⁷ PBMCs, b) cryopreservation medium at a percentage of about 80% (w/w), c) human serum albumin at a percentage of about 5% (w/w), wherein the pH of the formulation is about pH 7.4.

In some embodiments, the composition comprising PBMCs comprises: a) about 5×10⁶ PBMCs/mL, b) cryopreservation medium at a percentage of about 80% (w/w), c) human serum albumin at a percentage of about 5% (w/w), wherein the pH of the formulation is about pH 7.4.

### Constrictions Used in Generating Compositions of PBMCs Comprising HPV Antigen

In some embodiments, the invention provides compositions of PBMCs comprising a HPV antigen for stimulating an immune response. In some embodiments, the HPV antigen is delivered to the PBMCs intracellularly. Methods of introducing payloads to PBMCs are known in the art.

In some embodiments, the HPV antigen is introduced into the PBMCs by passing the cell through a constriction such that transient pores are introduced to the membrane of the cell thereby allowing the HPV antigen to enter the cell. Examples of constriction-based delivery of compounds into a cell are provided by WO 2013/059343, WO 2015/023982, WO 2016/070136, WO2017041050, WO2017008063, WO 2017/192785, WO 2017/192786, WO 2019/178005, WO 2019/178006, WO 2020/072833, WO 2020/154696, and WO 2020/176789.

In some embodiments, the HPV antigen and adjuvant are delivered into the PBMCs to produce the PBMCs of the invention by passing a cell suspension comprising the PBMCs through a constriction, wherein the constriction deforms the input PBMCs thereby causing a perturbation of the input PBMCs such that a HPV antigen and an adjuvant enter the perturbed input PBMCs. In some embodiments, the constriction is contained within a microfluidic channel. In some embodiments, multiple constrictions can be placed in parallel and/or in series within the microfluidic channel.

In some embodiments, the constriction within the microfluidic channel includes an entrance portion, a center point, and an exit portion. In some embodiments, the length, depth, and width of the constriction within the microfluidic channel can vary. In some embodiments, the width of the constriction within the microfluidic channel is a function of the diameter of the PBMCs cells. Methods to determine the diameter of PBMCs are known in the art; for example, high-content imaging, cell counters or flow cytometry.

In some embodiments, the width of the constriction is about 10% to about 99% of the mean diameter of the input PBMCs. In some embodiments, the width of the constriction is any one of about 10% to about 90%, about 10% to about 80%, about 10% to about 70%, about 20% to about 60%, about 40% to about 60%, about 30% to about 45%, about 50% to about 99%, about 50% to about 90%, about 50% to about 80%, about 50% to about 70%, about 60% to about 90%, about 60% to about 80%, or about 60% to about 70% of the mean diameter of the input PBMCs having the smallest diameter within the population of PBMCs. In some embodiments, the width of the constriction is any one of about 10% to about 90%, about 10% to about 80%, about 10% to about 70%, about 20% to about 60%, about 40% to about 60%, about 30% to about 45%, about 50% to about 99%, about 50% to about 90%, about 50% to about 80%, about 50% to about 70%, about 60% to about 90%, about 60% to about 80%, or about 60% to about 70% of the mean diameter of the input PBMCs.

In some embodiments of the constriction-based delivery of a HPV antigen to PBMCs, the width of the constriction is about 3 μm to about 15 μm. In some embodiments, the width of the constriction is about 3 μm to about 10 μm. In some embodiments, the width of the constriction is about 3 μm to about 6 μm. In some embodiments, the width of the constriction is about 4.2 μm to about 6 μm. In some embodiments, the width of the constriction is about 4.2 μm to about 4.8 μm. In some embodiments, the width of the constriction is about 3 μm to about 5 μm. In some embodiments, the width of the constriction is about 3 μm to about 3.5 μm. In some embodiments, the width of the constriction is about 3.5 μm to about 4 μm. In some embodiments, the width of the constriction is about 4 μm to about 4.5 μm. In some embodiments, the width of the constriction is about 3.2 μm to about 3.8 μm. In some embodiments, the width of the constriction is about 3.8 μm to about 4.3 μm. In some embodiments, the width of the constriction is about or less than any one of 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, 10.5 μm, 11 μm, 11.5 μm, 12 μm, 12.5 μm, 13 μm, 13.5 μm, 14 μm, 14.5 μm or 15 μm.

In some embodiments, the width of the constriction is about or less than any one of 3.0 μm, 3.1 μm, 3.2 μm, 3.3 μm, 3.4 μm, 3.5 μm, 3.6 μm, 3.7 μm, 3.8 μm, 3.9 μm, 4.0 μm, 4.1 μm, 4.2 μm, 4.3 μm, 4.4 μm, 4.5 μm, 4.6 μm, 4.7 μm, 4.8 μm, 4.9 μm, or 5.0 μm. In some embodiments, the width of the constriction is about 4.5 μm.

Examples of parameters that may influence the delivery of the compound into the PBMCs include, but are not limited to, the dimensions of the constriction, the entrance angle of the constriction, the surface properties of the constrictions (e.g., roughness, chemical modification, hydrophilic, hydrophobic, etc.), the operating flow speeds (e.g., cell transit time through the constriction), the cell concentration, the concentration of the compound in the cell suspension, buffer in the cell suspension, and the amount of time that PBMCs recover or incubate after passing through the constrictions can affect the passage of the delivered compound into the PBMCs. Additional parameters influencing the delivery of the compound into the PBMCs can include the velocity of the input PBMCs in the constriction, the shear rate in the constriction, the viscosity of the cell suspension, the velocity component that is perpendicular to flow velocity, and time in the constriction. In addition, multiple chips comprising channels in series and/or in parallel may impact delivery to PBMCs. Multiple chips in parallel may be useful to enhance throughput. Such parameters can be designed to control delivery of the compound. In some embodiments, the cell concentration ranges from about 10 to at least about 10¹² cells/mL or any concentration or range of concentrations therebetween. In some embodiments, delivery compound concentrations can range from about 10 ng/mL to about 1 g/mL or any concentration or range of concentrations therebetween. In some embodiments, delivery compound concentrations can range from about 1 pM to at least about 2 M or any concentration or range of concentrations therebetween.

In some embodiments, the concentration of HPV antigen incubated with the PBMCs is between about 0.01 μM and about 10 mM. For example, in some embodiments, the concentration of HPV antigen incubated with the PBMCs is any of less than about 0.01 μM, about 0.1 μM, about 1 μM, about 10 μM, about 100 μM, about 1 mM or about 10 mM. In some embodiments, the concentration of HPV antigen incubated with the PBMCs is greater than about 10 mM. In some embodiments, the concentration of HPV antigen incubated with the PBMCs is any of between about 0.01 μM and about 0.1 μM, between about 0.1 μM and about 1 μM, between about 1 μM and about 10 μM, between about 10 μM and about 100 μM, between about 100 μM and about 1 mM, or between 1 mM and about 10 mM. In some embodiments, the concentration of HPV antigen incubated with the PBMCs is between about 0.1 μM and about 1 mM. In some embodiments, the concentration of HPV antigen incubated with the PBMCs is between about 0.1 μM and about 10 μM. In some embodiments, the concentration of HPV antigen incubated with the PBMCs is 1 μM.

In some embodiments, the molar ratio of antigen to adjuvant incubated with the perturbed input PBMCs is any of between about 10000:1 to about 1:10000. For example, in some embodiments, the molar ratio of antigen to adjuvant incubated with the perturbed input PBMCs is about any of 10000:1, about 1000:1, about 100:1, about 10:1, about 1:1, about 1:10, about 1:100, about 1:1000, or about 1:10000. In some embodiments, the molar ratio of antigen to adjuvant incubated with the perturbed input P is any of between about 10000:1 and about 1000:1, between about 1000:1 and about 100:1, between about 100:1 and about 10:1, between about 10:1 and about 1:1, between about 1:1 and about 1:10, between about 1:10 and about 1:100, between about 1:100 and about 1:1000, between about 1:1000 and about 1:10000. In some embodiments, the molar ratio of antigen to adjuvant incubated with the perturbed input PBMCs is about 200:1. In some embodiments, the molar ratio of antigen to adjuvant incubated with the perturbed input PBMCs is about 20:1.

In some embodiments, the modified PBMCs comprise the adjuvant at a concentration between about 1 nM and about 1 mM. For example, in some embodiments, the modified PBMCs comprise the adjuvant at a concentration of any of less than about 0.01 μM, about 0.1 μM, about 1 μM, about 10 μM, about 100 μM, about 1 mM or about 10 mM. In some embodiments, the modified PBMCs comprise the adjuvant at a concentration of greater than about any of 10 mM. in some embodiments, the modified PBMCs comprise the adjuvant at a concentration of any of between about 1 nM to about 10 nM, about 0.1 μM and about 1 μM, between about 1 μM and about 10 μM, between about 10 μM and about 100 μM, between about 100 μM and about 1 mM, or between 1 mM and about 10 mM. In some embodiments, the modified PBMCs comprise the adjuvant at a concentration between about 0.1 μM and about 1 mM. In some embodiments, the Modified PBMCs comprise the adjuvant at a concentration of about 1 μM.

In some embodiments, the Modified PBMCs comprise the antigen at a concentration between about 1 nM and about 1 mM. For example, in some embodiments, the Modified PBMCs comprises the antigen at a concentration of any of less than about 0.01 μM, about 0.1 μM, about 1 μM, about 10 μM, about 100 μM, about 1 mM or about 10 mM. In some embodiments, the Modified PBMCs comprise the antigen at a concentration of greater than about any of 10 mM. in some embodiments, the Modified PBMCs comprise the antigen at a concentration of any of between about 1 nM to about 10 nM, about 0.1 μM and about 1 μM, between about 1 μM and about 10 μM, between about 10 μM and about 100 μM, between about 100 μM and about 1 mM, or between 1 mM and about 10 mM. In some embodiments, the Modified PBMCs comprise the antigen at a concentration between about 0.1 μM and about 1 mM. In some embodiments, the Modified PBMCs comprise the antigen at a concentration of about 1 μM.

In some embodiments, the molar ratio of antigen to adjuvant in the modified PBMCs is any of between about 10000:1 to about 1:10000. For example, in some embodiments, the molar ratio of antigen to adjuvant in the modified PBMCs is about any of 10000:1, about 1000:1, about 100:1, about 10:1, about 1:1, about 1:10, about 1:100, about 1:1000, or about 1:10000. In some embodiments, the molar ratio of antigen to adjuvant in the modified PBMCs is any of between about 10000:1 and about 1000:1, between about 1000:1 and about 100:1, between about 100:1 and about 10:1, between about 10:1 and about 1:1, between about 1:1 and about 1:10, between about 1:10 and about 1:100, between about 1:100 and about 1:1000, between about 1:1000 and about 1:10000. In some embodiments, the molar ratio of antigen to adjuvant in the modified PBMCs is about 200:1. In some embodiments, the molar ratio of antigen to adjuvant in the modified PBMCs is about 20:1.

### Conditioning of PBMCs

In some embodiments according to any one of methods described herein, the PBMCs comprising at least one HPV antigen are conditioned. In further embodiments, the PBMCs are matured. In some embodiments, the PBMCs are conditioned subsequent to constriction mediated delivery. In some embodiments, the PBMCs comprising the at least one HPV antigen are incubated with an adjuvant for a sufficient time for the cells comprising the constriction-delivered HPV antigens to condition, thereby generating a composition of conditioned cells comprising the at least one HPV antigen. In some embodiments, the PBMCs are conditioned subsequent to constriction-mediated delivery. In some embodiments, the PBMCs comprising the constriction-delivered HPV antigens are incubated with an adjuvant for a sufficient time for the PBMCs comprising the constriction-delivered mutated HPV antigens to condition, thereby generating a composition of conditioned PBMCs comprising the at least one HPV antigen. In some embodiments, the adjuvant is a CpG oligodeoxynucleotide (ODN), LPS, IFN-α, STING agonists, RIG-I agonists, poly I:C, R837, R848, a TLR3 agonist, a TLR4 agonist or a TLR 9 agonist. In some embodiments, the adjuvant is CpG ODN 2006 (also known as CpG 7909) (TCGTCGTTTTGTCGTTTTGTCGTT (SEQ ID NO:31)). In some embodiments, the adjuvant is CpG 7909. In some embodiments, the adjuvant is a CpG 7909 oligodeoxynucleotide (ODN).

In some aspects, there is provided a composition of conditioned PBMCs comprising at least one HPV antigen, prepared by a process comprising the steps of: a) passing a cell suspension comprising a population of input PBMCs through a cell-deforming constriction, wherein a width of the constriction is a function of the input PBMCs in the suspension, thereby causing perturbations of the input PBMCs large enough for the at least one HPV antigen to pass through to form perturbed input PBMCs; b) incubating the perturbed input PBMCs with the at least one HPV antigen for a sufficient time to allow the at least one HPV antigen to enter the perturbed PBMCs, thereby generating modified PBMCs comprising the at least one HPV antigen; and c) incubating the modified PBMCs comprising the constriction-delivered HPV antigens with an adjuvant for a sufficient time for the modified PBMCs comprising the constriction-delivered HPV antigens to condition, thereby generating the composition of conditioned PBMCs comprising the at least one HPV antigen. In some embodiments, the process further comprises isolating the modified PBMCs comprising the HPV antigen from the cell suspension before incubation with the adjuvant to condition the modified PBMCs. In some embodiments, the adjuvant is a CpG 7909 oligodeoxynucleotide (ODN).

In some embodiments, the PBMCs are conditioned prior to constriction-mediated delivery. In some embodiments, the PBMCs are incubated with an adjuvant for a sufficient time for the PBMCs to condition, thereby conditioning the PBMCs. In some embodiments, there is provided a composition of conditioned PBMCs comprising at least one HPV antigen, prepared by a process comprising the steps of: a) incubating PBMCs with an adjuvant for a sufficient time for the PBMCs to condition, thereby generating conditioned PBMCs; b) passing a cell suspension comprising the conditioned PBMCs through a cell-deforming constriction, wherein a width of the constriction is a function of a diameter of the PBMCs in the suspension, thereby causing perturbations of the PBMCs large enough for the at least one HPV antigen to pass through to form conditioned perturbed PBMCs; and c) incubating the conditioned perturbed PBMCs with the at least one HPV antigen for a sufficient time to allow the at least one HPV antigen to enter the conditioned perturbed PBMCs, thereby generating the conditioned PBMCs comprising the at least one HPV antigen. In some embodiments, the process further comprises isolating the conditioned PBMCs from the adjuvant before passing the conditioned PBMCs through a cell-deforming constriction. In some embodiments, the adjuvant is a CpG 7909 oligodeoxynucleotide (ODN).

In some embodiments according to any one of methods described herein, the PBMCs comprising the at least one HPV antigen are incubated with the adjuvant for about 1 to about 24 hours for the PBMCs to condition. In some embodiments, the PBMCs are incubated with the adjuvant for about 2 to about 10 hours for the PBMCs to condition. In some embodiments, the PBMCs are incubated with the adjuvant for about 3 to about 6 hours for the PBMCs to condition. In some embodiments, the PBMCs are incubated with the adjuvant for any one of about 1 hour, 2 hours, 3 hours, 3.5 hours, 4 hours, 4.5 hours, 5 hours, 5.5 hours, 6 hours, 8 hours, 12 hours, 16 hours, 20 hours, or 24 hours for the PBMCs to condition. In some embodiments, the PBMCs are incubated with the adjuvant for about 4 hours for the PBMCs to condition. In some embodiments, the PBMCs are incubated with the adjuvant at a temperature of about any one of: 4, 8, 12, 16, 20, 25, 26, 27, 28, 29, 30, 31, 32, 33, 34, 35, 36, 37, 38, 39 or 40° C. In some embodiments, the PBMCs are incubated with the adjuvant at about 37° C. In some embodiments, the PBMCs are incubated with the adjuvant for about 4 hours for the PBMCs to condition. In some embodiments, the PBMCs are incubated with the adjuvant at about 37° C. for about 4 hours for the PBMCs to condition. In some embodiments, the PBMCs are incubated with CpG 7909 for about 4 hours for the PBMCs to condition. In some embodiments, the PBMCs are incubated with the CpG 7909 at about 37° C. for about 4 hours for the PBMCs to condition. In some embodiments, the PBMCs are incubated with the CpG 7909 at a concentration of about any of 0.20 mg/mL, 0.25 mg/mL, 0.30 mg/mL, 0.35 mg/mL, 0.40 mg/mL, 0.45 mg/mL, or 0.50 mg/mL, or any concentration therebetween. In some embodiments, the PBMCs are incubated with the CpG 7909 at a concentration of about 0.35 mg/mL. In some embodiments, the PBMCs are incubated with the CpG 7909 at a concentration of about 0.35 mg/mL and at about 37° C. for about 4 hours for the PBMCs to condition.

In some embodiments, there is provided a conditioned plurality of PBMCs comprising at least one HPV antigen, prepared by incubating the plurality of PBMCs comprising the at least one HPV antigen with an adjuvant for a sufficient time for the PBMCs to condition, thereby generating the conditioned plurality of PBMCs comprising the at least one HPV antigen. In some embodiments, there is provided a conditioned plurality of PBMCs comprising at least one HPV antigen, prepared by incubating the plurality of PBMCs with an adjuvant for a sufficient time for the PBMCs to condition prior to introducing the at least one HPV antigen to the PBMCs, thereby generating the conditioned plurality of PBMCs comprising the at least one HPV antigen.

In some embodiments according to any of the conditioned plurality of PBMCs described herein, the plurality of PBMCs is incubated with the adjuvant for about 1 to about 24 hours for the PBMCs to condition. In some embodiments, the plurality of PBMCs is incubated with the adjuvant for about 2 to about 10 hours for the PBMCs to condition. In some embodiments, the plurality of PBMCs is incubated with the adjuvant for about 3 to about 6 hours for the PBMCs to condition. In some embodiments, the plurality of PBMCs is incubated with the adjuvant for any one of about 1 hour, 2 hours, 3 hours, 3.5 hours, 4 hours, 4.5 hours, 5 hours, 5.5 hours, 6 hours, 8 hours, 12 hours, 16 hours, 20 hours, or 24 hours for the PBMCs to condition. In some embodiments, the plurality of PBMCs is incubated with the adjuvant for about 4 hours for the PBMCs to condition. In some embodiments, the PBMCs are incubated with the adjuvant at a temperature of about any one of: 4, 8, 12, 16, 20, 25, 26, 27, 28, 29, 30, 31, 32, 33, 34, 35, 36, 37, 38, 39 or 40° C. In some embodiments, the PBMCs are incubated with the adjuvant at about 37° C. In some embodiments, the PBMCs comprising the at least one HPV antigen are conditioned by a process comprising incubating the PBMCs with an adjuvant for about 2 hours to about 10 hours, about 3 hours to about 6 hours, or about 4 hours at about 37° C. for the PBMCs to condition.

In some embodiments according to any one of the conditioned plurality of PBMCs described herein, one or more co-stimulatory molecules are upregulated in the conditioned plurality of modified PBMCs compared to an unconditioned plurality of modified PBMCs. In some embodiments, one or more co-stimulatory molecules are upregulated in a subpopulation of cells in the conditioned plurality of modified PBMCs compared to the subpopulation of cells in an unconditioned plurality of modified PBMCs. In some embodiments, one or more co-stimulatory molecules are upregulated in the B cells of the conditioned plurality of modified PBMCs compared to the B cells in an unconditioned plurality of modified PBMCs. In some embodiments, the co-stimulatory molecule is CD80 and/or CD86. In some embodiments, the co-stimulatory molecule is CD86. In some embodiments, the CD80 and/or CD86 is upregulated in the B cells of the conditioned plurality of modified PBMCs by more than about 1.2-fold, 1.5-fold, 1.8-fold, 2-fold, 3-fold, 4-fold, 5-fold, 8-fold, or more than 10-fold compared to the B cells in an unconditioned plurality of modified PBMCs. In some embodiments, the CD80 and/or CD86 is unregulated in the B cells of the conditioned plurality of modified PBMCs by any of about 1.2-fold to about 1.5-fold, about 1.5-fold to about 1.8-fold, about 1.8-fold to about 2-fold, about 2-fold to about 3-fold, about 3-fold to about 4-fold, about 4-fold to about 5-fold, about 5-fold to about 8-fold, about 8-fold to about 10-fold, about 10-fold to about 20-fold, about 20-fold to about 50-fold, about 50-fold to about 100-fold, about 100-fold to about 200-fold, about 200-fold to about 500-fold, or more than about 500-fold compared to the B cells in an unconditioned plurality of modified PBMCs. In some embodiments, the expression of one or more of IFN-γ, IL-6, MCP-1, MIP-1β, IP-10, or TNF-α is increased in the conditioned plurality of modified PBMCs compared to an unconditioned plurality of modified PBMCs. In some embodiments, the expression of one or more of IFN-γ, IL-6, MCP-1, MIP-1β, IP-10, or TNF-α is increased a subpopulation of cells in the conditioned plurality compared to the subpopulation of cells in an unconditioned plurality of modified PBMCs. In some embodiments, the expression of one or more of IFN-γ, IL-6, MCP-1, MIP-1β, IP-10, or TNF-α is increased by about 1.2-fold, 1.5-fold, 1.8-fold, 2-fold, 3-fold, 4-fold, 5-fold, 8-fold, or more than 10-fold in the conditioned plurality of modified PBMCs compared to an unconditioned plurality of modified PBMCs. In some embodiments, the expression of one or more of IFN-γ, IL-6, MCP-1, MIP-1β, IP-10, or TNF-α is increased by any of about 1.2-fold to about 1.5-fold, about 1.5-fold to about 1.8-fold, about 1.8-fold to about 2-fold, about 2-fold to about 3-fold, about 3-fold to about 4-fold, about 4-fold to about 5-fold, about 5-fold to about 8-fold, about 8-fold to about 10-fold, about 10-fold to about 20-fold, about 20-fold to about 50-fold, about 50-fold to about 100-fold, about 100-fold to about 200-fold, about 200-fold to about 500-fold, or more than about 500-fold in the conditioned plurality of modified PBMCs compared to an unconditioned plurality of modified PBMCs.

### Systems and Kits

In some aspects, the invention provides a system comprising one or more of the constriction, a PBMC cell suspension, HPV antigens or adjuvants for use in the methods disclosed herein. The system can include any embodiment described for the methods disclosed above, including microfluidic channels or a surface having pores to provide cell-deforming constrictions, cell suspensions, cell perturbations, delivery parameters, compounds, and/or applications etc. In some embodiment, the cell-deforming constrictions are sized for delivery to PBMCs. In some embodiments, the delivery parameters, such as operating flow speeds, cell and compound concentration, velocity of the cell in the constriction, and the composition of the cell suspension (e.g., osmolarity, salt concentration, serum content, cell concentration, pH, etc.) are optimized for maximum response of a compound for suppressing an immune response or inducing tolerance.

Also provided are kits or articles of manufacture for use in treating individuals with a cancer associated with HPV. In some embodiments, the kit comprises PBMCs comprising intracellularly a HPV antigen and intracellularly an adjuvant. In some embodiments, the kit comprises one or more of the constriction, a PBMC suspension, HPV antigens or adjuvants for use in generating PBMCs for use in treating an individual with a disease associated with HPV, such as cancer. In some embodiments, the kits comprise the compositions described herein (e.g. a microfluidic channel or surface containing pores, cell suspensions, and/or compounds) in suitable packaging. Suitable packaging materials are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

The invention also provides kits comprising components of the methods described herein and may further comprise instructions for performing said methods treat an individual with a cancer associated with HPV and/or instructions for introducing at least one HPV antigen into PBMCs. The kits described herein may further include other materials, including other buffers, diluents, filters, needles, syringes, and package inserts with instructions for performing any methods described herein; e.g., instructions for treating an individual with a cancer associated with HPV or instructions for generating PBMCs to contain intracellularly at least one HPV antigen.

### EXEMPLARY EMBODIMENTS

Embodiment 1. A method for treating a human papilloma virus (HPV)-associated cancer in an individual, the method comprising:

administering an effective amount of a composition comprising peripheral blood mononuclear cells (PBMCs) to the individual, wherein the PBMCs comprise at least one HPV antigen delivered intracellularly, and

administering an effective amount of an antagonist of CTLA-4 and/or an antagonist of PD-1/PD-L1 to the individual.

Embodiment 2. The method of embodiment 1, wherein the antagonist of CTLA4 is an antibody that binds CTLA4.

Embodiment 3. The method of embodiment 1 or 2, wherein the antagonist of PD-1/PD-L1 is an antibody that binds PD-1 or an antibody that binds PD-L1.

Embodiment 4. The method of any one of embodiments 1-3, wherein an antibody that binds CTLA-4 and an antibody that binds PD-1 are administered to the individual.

Embodiment 5. The method of any one of embodiments 1-3, wherein an antibody that binds CTLA-4 is administered to the individual and an antibody that binds PD-L1 is administered to the individual.

Embodiment 6. The method of any one of embodiments 2-5, wherein the antibody that binds CTLA-4 is ipilimumab.

Embodiment 7. The method of any one of embodiments 3, 4 and 6, wherein the antibody that binds PD-1 is nivolumab.

Embodiment 8. The method of any one of embodiments 3, 4 and 6, wherein the antibody that binds PD-1 is pembrolizumab.

Embodiment 9. The method of any one of embodiments 3, 4 and 6, wherein the antibody that binds PD-L1 is atezolizumab.

Embodiment 10. A method for treating a HPV+ recurrent, locally advanced or metastatic tumor in an individual, the method comprising administering an effective amount of a composition comprising peripheral blood mononuclear cells (PBMCs) to the individual, wherein the PBMCs comprise at least one HPV antigen delivered intracellularly.

Embodiment 11. The method of embodiment 10, wherein the composition comprising PBMCs is administered in conjunction with one or more immune checkpoint inhibitors.

Embodiment 12. The method of embodiment 11, wherein the checkpoint inhibitor is an antagonist of CTLA-4 and/or an antagonist of PD-1/PD-L1 to the individual.

Embodiment 13. The method of embodiment 11 or 12, wherein the one or more immune checkpoint inhibitor is an antibody that binds PD-L1, CTLA-4, or PD-1.

Embodiment 14. The method of any or of embodiments 11-13, wherein the composition comprising PBMCs is administered in conjunction with an antibody that binds CTLA-4 and an antibody that binds PD 1.

Embodiment 15. The method of embodiment 13, wherein the antibody that binds PD-L1 is atezolizumab.

Embodiment 16. The method of any one of embodiments 13-15, wherein the antibody that binds CTLA-4 is ipilimumab.

Embodiment 17. The method of any one of embodiments 13, 14 and 16, wherein the antibody that binds PD-1 is nivolumab.

Embodiment 18. The method of any one of embodiments 13, 14 and 16, wherein the antibody that binds PD-1 is pembrolizumab.

Embodiment 19. The method of any one of embodiments 1-18, wherein the at least one HPV antigen is an HPV-16 antigen or an HPV-18 antigen.

Embodiment 20. The method of embodiment 19, wherein the at least one HPV antigen comprises a peptide derived from HPV E6 and/or E7.

Embodiment 21. The method of any one of embodiments 1-20, wherein the at least one HPV antigen comprises an HLA-A2-restricted peptide derived from HPV E6 and/or E7.

Embodiment 22. The method of embodiment 21, wherein the HLA-A2-restricted peptide comprises the amino acid sequence of any one of SEQ ID NOs:1-4.

Embodiment 23. The method of any one of embodiments 1-20, wherein the at least one HPV antigen comprises the amino acid sequence of any one of SEQ ID NOs:18-25.

Embodiment 24. The method on any one of embodiments 1-23, wherein the PBMCs comprise an antigen comprising the amino acid sequence of SEQ ID NO:19 and an antigen comprising the amino acid sequence of SEQ ID NO:23.

Embodiment 25. The method of any one of embodiments 1-24, where the individual is human.

Embodiment 26. The method of any one of embodiments 1-25, wherein the individual is positive for HLA-A*02.

Embodiment 27. The method of any one of embodiments 1-26, wherein the PBMCs are positive for HLA-A*02.

Embodiment 28. The method of any one of embodiments 1-27, where the PBMCs are autologous to the individual.

Embodiment 29. The method of any one of embodiments 1-28, wherein the individual is positive for human immunodeficiency virus (HIV).

Embodiment 30. The method of any one of embodiments 1-29, wherein the HPV-associated cancer is head and neck cancer, cervical cancer, anal cancer or esophageal cancer.

Embodiment 31. The method of any one of embodiments 1-30, wherein the composition comprising PBMCs are administered intravenously.

Embodiment 32. The method of any one of embodiments 1-9 and 12-31, wherein the antagonist of CTLA-4 and/or antagonist of PD-1/PD-L1 is administered intravenously, orally, or subcutaneously.

Embodiment 33. The method of any one of embodiments 2-9 and 13-32, wherein the antibody that binds CTLA-4 and/or the antibody that binds PD-1 and/or the antibody that binds PD-L1 is administered intravenously.

Embodiment 34. The method of any one of embodiments 1-33, wherein the effective amount of PBMCs comprising the at least one HPV antigen is about 0.5×10⁶ cells/kg to about 5.0×10⁶ cells/kg.

Embodiment 35. The method of any one of embodiments 6-9 and 16-34, wherein the effective amount of ipilimumab is about 1 mg/kg to about 3 mg/kg.

Embodiment 36. The method of any one of embodiments 7 and 17-35, wherein the effective amount of nivolumab is about 360 mg.

Embodiment 37. The method of any one of embodiments 9, 15, 16, and 19-36, wherein the effective amount of atezolizumab is about 1200 mg.

Embodiment 38. The method of any one of embodiments 1-37, wherein the composition comprising the PBMCs is delivered on day 1 of a three-week cycle.

Embodiment 39. The method of any one of embodiments 1-38, wherein the composition comprising the PBMCs is further administered on day 2 of a first three-week cycle.

Embodiment 40. The method of embodiment 38 or 39, wherein about 0.5×10⁶ cells/kg, about 2.5×10⁶ cells/kg about 5.0×10⁶ cells/kg are administered on day 1 of each three-week cycle.

Embodiment 41. The method of embodiment 39 or 40, wherein about 0.5×10⁶ cells/kg, about 2.5×10⁶ cells/kg or about 5.0×10⁶ cells/kg are administered on day 2 of the first three-week cycle.

Embodiment 42. The method of any one of embodiments 2-9 and 13-41, wherein the antibody that binds CTLA 4 and/or the antibody that binds PD-1 and/or the antibody that binds PD-L1 is administered once per three-week cycle.

Embodiment 43. The method of any one of embodiments 38-42, wherein the antibody that binds CTLA-4 is administered on day 1 of each three-week cycle.

Embodiment 44. The method of any one of embodiments 38-42, wherein the antibody that binds CTLA-4 is administered once per two three-week cycles.

Embodiment 45. The method of any one of embodiments 38-44, wherein the antibody that binds CTLA-4 is ipilimumab, wherein the ipilimumab is administered at a dose of about 3 mg/kg.

Embodiment 46. The method of any one of embodiments 42-45, wherein the antibody that binds PD-1 is administered on day 8 of the first three-week cycle and day 1 of each subsequent cycle.

Embodiment 47. The method of embodiment 46, wherein the antibody that binds PD-1 is nivolumab, wherein the nivolumab is administered at a dose of about 360 mg.

Embodiment 48. The method of any one of embodiments 38-42, wherein the antibody that binds CTLA-4 is ipilimumab, wherein the ipilimumab is administered on day 1 of the first three-week cycle of two three-week cycles at a dose of about 1 mg/kg and the antibody that binds PD-1 is administered on day 8 of the first three-week cycle and day 1 of each subsequent cycle at a dose of about 360 mg.

Embodiment 49. The method of any one of embodiments 38-45, wherein the antibody that binds PD-L1 is administered on day 8 of the first three-week cycle and day 1 of each subsequent cycle.

Embodiment 50. The method of embodiment 48, wherein the antibody that binds PD-L1 is atezolizumab, wherein the atezolizumab is administered at a dose of about 1200 mg.

Embodiment 51. The method of any one of embodiments 1-49, wherein the composition comprising PBMCs is administered to the individual for at least about three months, six months, nine months or one year.

Embodiment 52. The method of any one of embodiments 1-51, wherein the composition comprising PBMCs comprises

a) about 5×10⁶ PBMCs to about 5×10⁷ PBMCs,

b) cryopreservation medium at a percentage of about 40% to about 60% (w/w),

c) hypothermic preservation medium at a percentage of about 25% to about 35% (w/w), and

d) human serum albumin about 3% to about 8% (w/w),

wherein the pH of the formulation is about pH 6.0 to about pH 8.5.

Embodiment 53. The method of any one of embodiments 1-52, wherein the composition comprising PBMCs comprises

a) about 2.75×10⁷ PBMCs,

b) cryopreservation medium at a percentage of about 50% (w/w),

c) hypothermic preservation medium at a percentage of about 30% (w/w), and

d) human serum albumin at a percentage of about 5% (w/w),

wherein the pH of the formulation is about pH 7.4.

Embodiment 54. The method of any one of embodiments 1-53, wherein the composition comprising PBMCs comprises

a) about 1×10⁶ PBMCs/mL to about 1×10⁷ PBMCs/mL,

b) cryopreservation medium at a at a percentage of about 40% to about 60% (w/w),

c) hypothermic preservation medium at a percentage of about 25% to about 35% (w/w), and

d) human serum albumin at a percentage of about 3% to about 8% (w/w),

wherein the pH of the formulation is about pH 6.0 to about pH 8.5.

Embodiment 55. The method of any one of embodiments 1-54, wherein the composition comprising PBMCs comprises

a) about 5×10⁶ PBMCs/mL,

b) cryopreservation medium at a percentage of about 50% (w/w),

c) hypothermic preservation medium at a percentage of about 30% (w/w), and

d) human serum albumin at a percentage of about 5% (w/w),

wherein the pH of the formulation is about pH 7.4.

Embodiment 56. The method of any one of embodiments 1-51, wherein the composition comprising PBMCs comprises

a) about 5×10⁶ PBMCs to about 5×10⁷ PBMCs,

b) cryopreservation medium at a percentage of about 65% to about 95% (w/w),

c) human serum albumin at a percentage of about 3% to about 8% (w/w),

wherein the pH of the formulation is about pH 6.0 to about pH 8.5.

Embodiment 57. The method of any one of embodiments 1-51, wherein the composition comprising PBMCs comprises

a) about 1×10⁶ PBMCs/mL to about 1×10⁷ PBMCs/mL,

b) cryopreservation medium at a percentage of about 65% to about 95% (w/w),

c) human serum albumin at a percentage of about 3% to about 8% (w/w),

wherein the pH of the formulation is about pH 6.0 to about pH 8.5.

Embodiment 58. The method of any one of embodiments 1-51, wherein the composition comprising PBMCs comprises

a) about 2.5×10⁷ PBMCs,

b) cryopreservation medium at a percentage of about 80% (w/w),

c) human serum albumin at a percentage of about 5% (w/w),

wherein the pH of the formulation is about pH 7.4.

Embodiment 59. The method of any one of embodiments 1-51, wherein the composition comprising PBMCs comprises

a) about 5×10⁶ PBMCs/mL,

b) cryopreservation medium at a percentage of about 80% (w/w),

c) human serum albumin at a percentage of about 5% (w/w),

wherein the pH of the formulation is about pH 7.4.

Embodiment 60. The method of any one of embodiments 52-59, wherein the cryopreservation medium is CryoStor® CS10.

Embodiment 61. The method of any one of embodiments 52-55, wherein the hypothermic preservation medium is HypoThermasol® FRS.

Embodiment 62. The method of any one of embodiments 1-61, wherein the PBMCs comprises two or more of T cells, B cells, NK cells or monocytes.

Embodiment 63. The method of any one of embodiments 1-62, wherein the PBMCs comprises T cells, B cells, NK cells and monocytes.

Embodiment 64. The method of any one of embodiments 1-63, wherein

(a) about 25% to about 80% of the PBMCs are T cells;

(b) about 1.5% to about 30% of the PBMCs are B cells;

(c) about 3.0% to about 20% of the PBMCs are NK cells; or

(d) about 4.0% to about 45% of the PBMCs are monocytes.

Embodiment 65. The method of any one of embodiments 1-64, wherein the PBMCs comprising the at least one HPV antigen are prepared by a process comprising:

a) passing a cell suspension comprising a population of input PBMCs through a cell-deforming constriction, wherein a diameter of the constriction is a function of a diameter of the input PBMCs in the suspension, thereby causing perturbations of the input PBMCs large enough for the at least one HPV antigen to pass through to form perturbed input PBMCs; and

b) incubating the population of perturbed input PBMCs with the at least one HPV antigen for a sufficient time to allow the antigen to enter the perturbed input PBMCs, thereby generating the PBMCs comprising the at least one HPV antigen.

Embodiment 66. The method of embodiment 65, wherein the diameter of the constriction is about 4.2 μm to about 6 μm or about 4.2 μm to about 4.8 μm.

Embodiment 67. The method of any one of embodiments 1-66, wherein the PBMCs comprising the at least one HPV antigen are conditioned.

Embodiment 68. The method of embodiment 67, wherein the PBMCs comprising the at least one HPV antigen are conditioned by a process comprising incubating the PBMCs with an adjuvant for about 2 hours to about 10 hours, about 3 hours to about 6 hours, or about 4 hours at about 37° C. for the PBMCs to condition.

Embodiment 69. The method of embodiment 68, wherein the adjuvant is a CpG oligodeoxynucleotide (ODN), LPS, IFN-α, STING agonists, RIG-I agonists, poly I:C, R837, R848, a TLR3 agonist, a TLR4 agonist or a TLR 9 agonist.

Embodiment 70. The method of embodiment 68 or 69, wherein the adjuvant is a CpG 7909 oligodeoxynucleotide (ODN).

### EXAMPLES

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### Example 1. Phase I Study of Safety and Tolerability of SQZ-PBMC-HPV

A Phase 1 open-label, multicenter study of the safety and tolerability, antitumor activity, and immunogenic and pharmacodynamic effects of SQZ-PBMC-HPV as monotherapy and in combination with (1) atezolizumab, (2) ipilimumab, (3) nivolumab, and (4) nivolumab plus ipilimumab, in HLA A*02+ patients with recurrent, locally advanced or metastatic HPV16+ solid tumors is conducted.

SQZ PBMC HPV is an antigen presenting peripheral blood mononuclear cell (PBMC) product for a treatment for human papillomavirus (HPV) strain 16 positive (HPV16+) cancer in human leukocyte antigen (HLA) serotype within the HLA-A serotype group positive (HLA-A*02+) patients. SQZ PBMC HPV consists of autologous PBMCs presenting immunogenic epitopes of the E6 and E7 proteins of HPV16. The PBMCs consist primarily of T cells, monocytes, natural killer cells, and B cells. The PBMC-HPV drug substance is formulated into SQZ PBMC HPV, which contains a cryogenic medium, and then cryopreserved. SQZ PBMC HPV is stored under cryopreservation and thawed at the time of use.

SQZ-PBMC-HPV drug substances consists of autologous PBMCs that have synthetic long peptides (SLPs) containing HLA-A*02-restricted E6 and E7 epitopes of HPV16 delivered cytosolically during the manufacturing process.

E6 SLP:
(SEQ ID NO: 19)
QLCTELQTTIHDIILECVYCKQQLL
E7 SLP:
(SEQ ID NO: 23)
QLCTELQTYMLDLQPETTYCKQQLL

The PBMC-HPV cells are then matured with CpG 7909, a CpG oligodeoxynucleotide. This maturation of PBMC HPV during the manufacturing process facilitates endogenous T cells to be stimulated with antigen in an appropriate context.

### Overview

The study population consists of patients who are HLA-A*02+ with advanced-stage HPV16+ solid tumors (head and neck, cervical cancer, and other tumor types). HLA A*02+ status and HPV16+ tumor status must be confirmed via laboratory reports, and all eligibility criteria must be met, prior to the patient's leukapheresis. Patients with locally confirmed HPV16+ status may have central confirmation done from the fresh tumor biopsy collected at Screening if documentation of laboratory accreditation is deemed by the Sponsor to be insufficient.

Eligible patients undergo a single leukapheresis at the study sites. The leukapheresis product is sent to the manufacturer for manufacture of each patient's personalized autologous cellular therapy. Frozen vials of SQZ PBMC HPV are then sent to the study sites for administration.

This study is conducted in three parts, with Part 1 consisting of dose escalation to determine the safety profile and RP2D of SQZ-PBMC-HPV monotherapy. Part 2 of the study will evaluate the safety and preliminary efficacy of SQZ-PBMC-HPV when combined with immune checkpoint inhibitors. Part 3 will evaluate the SQZ-PBMC-HPV monotherapy RP2D in four dose expansion cohorts. Up to 29 patients will be enrolled in each Part 3 cohort.

The four dose expansion cohorts will be evaluated using an optimal Simon 2-stage design. In the first stage, up to 10 HIV negative patients are enrolled. If at least one response is observed in those 10 patients, an additional 19 patients are enrolled, for a total of 29.

In all cohorts, SQZ PBMC-HPV are administered at 3 week intervals for a maximum of 1 year or until the SQZ-PBMC-HPV supply is exhausted or until treatment discontinuation criteria are met, whichever comes first.

All patients in Part 1 and Part 2 are observed for at least 4 hours after each administration of SQZ PBMC HPV. In addition, the first 2 patients in each cohort will undergo a minimum 23 hours of observation following the first administration of SQZ PBMC-HPV.

Tumor assessments are performed throughout the study per RECIST 1.1 and iRECIST until disease progression, unacceptable toxicity, withdrawal of consent, death, or for 2 years from the date of the first administration of SQZ PBMC HPV, whichever occurs first. Patients who experience disease progression per RECIST 1.1 may continue dosing if considered in their best interest by the treating Investigator to allow for confirmation of disease progression; i.e., iCPD according to iRECIST (Seymour et al, 2017).

After the last dose of investigational product, follow-up visits will occur to monitor safety and tolerability and evaluate overall survival.

### Part 1: Escalation Phase (SQZ-PBMC-HPV Monotherapy)

Planned dose cohorts for the Escalation Phase are shown in Table 1. While the traditional 3+3 design is intended to assess safety and tolerability, it may be prudent to treat up to 6 additional patients in a cohort to further investigate safety and tolerability, immunogenic effects, and antitumor activity. There will be a maximum of 12 patients per cohort in this modified 3+3 design.

For the monotherapy RP2D regimen, the DLT assessment in all cohorts is completed. The RP2D regimen is selected based on review of all available safety, tolerability, immunogenic, and other pharmacodynamic and antitumor data.

Once the RP2D regimen is defined, Part 2 (Combination Safety Phase) and Part 3 (Monotherapy Dose Expansion Phase) is initiated.

TABLE 1
Summary of Monotherapy Cohorts Planned During the Escalation Phase

| | Dose of | | Potential Number of |
|---|---|---|---|
| | SQZ-PBMC-HPV | Total | Patients/Cohort based |
| Cohort | live cells/kg | Doses^{a} | on DLT |
| 1 | 0.5 × 10⁶ | ≥3 | 3-12 |
| 2 | 2.5 × 10⁶ | ≥3 | 3-12 |
| 3 | 2.5 × 10⁶ | ≥4^{b} | 3-12 |

DLT = dose-limiting toxicity; iRECIST = modified RECIST criteria for incorporation into solid tumor studies of immunotherapeutics; RECIST = Response Evaluation Criteria for Solid Tumours version 1.1
^{a}Dosing with SQZ-PBMC-HPV will continue every 3 weeks until treatment discontinuation criteria are met, or until the SQZ-PBMC-HPV supply is exhausted, or for a maximum of 1 year, whatever comes first. Patients who experience disease progression per RECIST 1.1 may continue dosing if considered in their best interest by the treating Investigator to allow for confirmation of disease progression; ie, iCPD according to iRECIST (Seymour et al. 2017).
^{b}In Cycle 1, patients will receive SQZ-PBMC-HPV on Days 1 and 2.

Tumor assessments are performed throughout the study until disease progression per RECIST 1.1 or iRECIST. After the last dose of study drug, follow-up visits will occur to monitor safety and tolerability, immunogenic effects, and for tumor assessments.

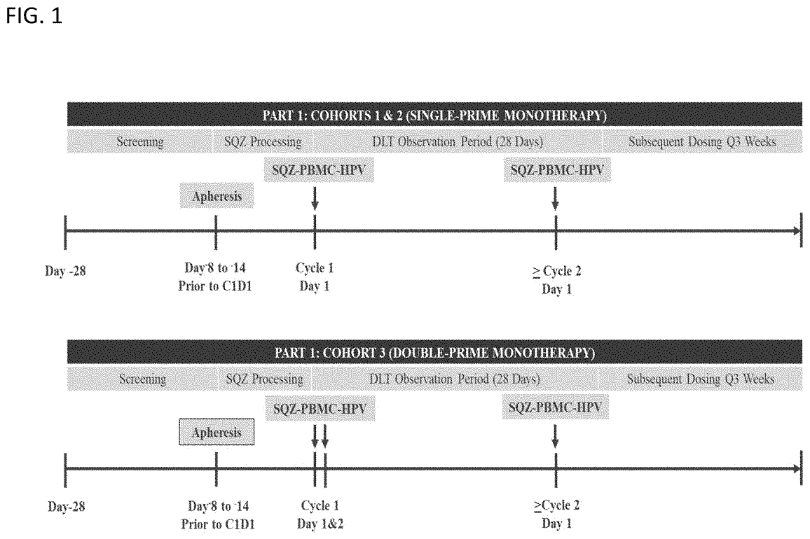

Patients are evaluated in a modified 3+3 dose-escalation design. At least 2 dose levels (0.5×10⁶ live cells/kg and 2.5×10⁶ live cells/kg) of SQZ PBMC HPV are evaluated as monotherapy (Cohorts 1, 2, and 3). Patients in Cohorts 1 and 2 receive SQZ-PBMC-HPV on Day 1 of each 21-day cycle (single priming); patients in Cohort 3 receive SQZ-PBMC-HPV on Days 1 and 2 of Cycle 1 (double priming) and Day 1 of each subsequent cycle (see FIG. 1). In each cohort, the first 2 patients must complete Cycle 1 Day 8 before additional patients in the cohort can be treated in that cohort.

Patients must have sufficient autologous drug product to achieve at least 3 full SQZ-PBMC-HPV dose administrations to be dosed in a cohort or will get assigned to a lower dose cohort.

While the traditional 3+3 design is intended to assess safety and tolerability, it may be prudent to treat up to 6 additional patients in a cohort to further investigate safety and tolerability, immunogenic effects, and antitumor activity. There are a maximum of 12 patients per cohort in this modified 3+3 design.

The RP2D regimen is selected based on review of all available safety, tolerability, immunogenic, and other pharmacodynamic and antitumor data. For the monotherapy RP2D regimen, the DLT assessment in all cohorts are completed.

Patients are enrolled in a staggered manner across all investigative sites, meaning no more than 1 patient in a cohort receives the first administration of SQZ PBMC-HPV within 1 week.

Patients are monitored for the occurrence of DLTs for 28 days after the first dose of SQZ PBMC-HPV in monotherapy cohorts. Following the modified 3+3 rules, the minimum number of patients needed to confirm a cohort as safe with respect to DLTs is 0 DLTs in 3 patients, ≤1 DLT in 6 patients, ≤2 DLTs in 9 patients or ≤3 DLTs in 12 patients.

The dose regimens are listed below, but intermediate dose levels may be selected if necessary, based on review of available safety data:
5×10⁶ live cells/kg (single prime)
5×10⁶ live cells/kg (double prime)
A lower dose level (single or double prime)

Dose escalation or expansion to 6 to 12 patients is considered after the first 3 patients at a given dose level have completed the DLT observation period and are found to be evaluable for safety upon review of safety data. The DLT observation period is defined as 28 days for Part 1.

If there are no DLTs observed in any of the first 3 enrolled patients at a given dose level through the DLT observation period, then the next higher dose level cohort is opened for enrollment. If 1 of the first 3 patients experiences DLT, then 3 additional patients are enrolled (total of 6 evaluable patients at the same dose level). If >1 of the first 3 patients or ≥2 of 6 patients experience DLT, then no further dose escalation will be considered and this is the maximum administered dose (MAD). The RP2D may be a previously evaluated, lower dose level or an alternative intermediate dose level may be selected for further evaluation. The RP2D determination is made based on safety data from at least 6 patients. The RP2D is further evaluated in Part 2 (Combination Safety Phase) and Part 3 (Monotherapy Dose Expansion Phase) of the study. Alternatively, the RP2D is declared, based on pharmacodynamic assessment, where it is determined that the maximum biologic effect has been achieved, and that patients would not benefit from further dose escalation.

A patient is considered non-evaluable if, for any reason other than safety, the patient is unable to complete the DLT observation period or if the pharmacodynamic assessments are insufficient to define the biological effect of study treatment. Patients in Part 1 considered non-evaluable may be replaced after consultation between the investigators and Sponsor.

Adverse events that develop after any administered dose should have resolved to <Grade 2 at time of subsequent administrations. Similarly, AESIs that develop after any administered dose should have resolved to <Grade 2 at time of subsequent administration. In Cohort 3, if these retreatment criteria are met, the second SQZ PBMC-HPV administration should be given during the ≥23-hour observation period (i.e., between 16 and 24 hours post first dose). Patients will be observed for a minimum of 4 hours after the second priming administration. The minimum interval between the 2 administrations should be 16 hours.

### Part 2: Combination Safety Phase (SQZ-PBMC-HPV+Checkpoint Inhibitor[s])

Once the SQZ-PBMC-HPV monotherapy RP2D is defined, the Combination Safety Phase is initiated. The SQZ-PBMC-HPV dose evaluated during Combination Safety exploration is selected based on review of all available safety, tolerability, immunogenic, and other pharmacodynamic and antitumor data.

The cohorts are defined by the SQZ PBMC HPV RP2D and the combination partner. SQZ-PBMC-HPV is administered in the RP2D in Cohorts 4, 5, 6, and 7.

Cohort 4: SQZ-PBMC-HPV (RP2D) plus atezolizumab (1200 mg every 3 weeks)

Cohort 5: SQZ-PBMC-HPV (RP2D) plus ipilimumab (3 mg/kg every 3 weeks for a maximum of 4 doses if tolerability allows)

Cohort 6: SQZ-PBMC-HPV (RP2D) plus nivolumab (360 mg every 3 weeks)

Cohort 7 (contingent on the safety assessment of 6 patients each treated in Cohorts 5 and 6): SQZ-PBMC-HPV (RP2D) plus nivolumab (360 mg every 3 weeks) and ipilimumab (1 mg/kg every 6 weeks)

Enrollment in Part 2 begins with Cohorts 4, 5, and 6. Once 6 patients each in Cohorts 5 and 6 are enrolled and successfully complete the 42-day DLT evaluation period; i.e., <33% of patients experience DLT, then Cohort 7 opens for enrollment. Based on the available safety data from both cohorts, it is decided whether the SQZ-PBMC-HPV dose regimen selected for Cohorts 5 and 6 are selected for Cohort 7 or whether to start at lower dose regimen. If a lower dose of SQZ-PBMC-HPV for Cohort 7 is decided, 6 patients are enrolled initially and observed for 42 days. If the SSC deems the combination safe, with <33% of patients experiencing DLT, the dose of SQZ-PBMC-HPV may be escalated to the full monotherapy RP2D and enrollment may continue until up to 12 patients have been enrolled.

All patients are evaluated for safety and tolerability as well as preliminary evidence of antitumor response.

Cohort 4—SQZ-PBMC-HPV plus Atezolizumab

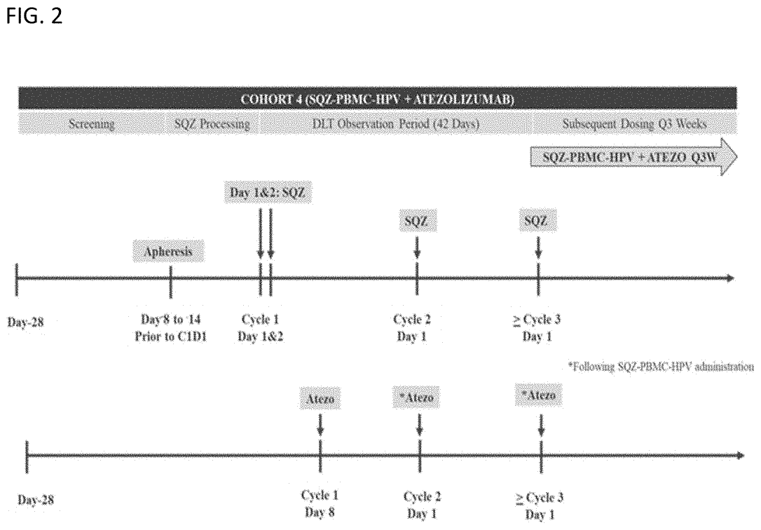

In Cycle 1, SQZ-PBMC-HPV is administered IV in accordance with the RP2D determined in Part 1; i.e., either as double priming on Days 1 and 2, or as a single-prime dose on Day 1. Atezolizumab 1200 mg is administered IV over 60 minutes on Day 8 of Cycle 1. In subsequent cycles, atezolizumab is administered on Day 1 of each 3-week cycle, following the administration of SQZ PBMC HPV and continued for a maximum of 2 years or until 1 of the criteria for treatment discontinuation are met (FIG. 2). SQZ PBMC HPV is administered in 3-week cycles until treatment discontinuation criteria are met, or the SQZ-PBMC-HPV supply has been exhausted, or for a maximum of 1 year, whichever comes first.

Cohort 5—SQZ-PBMC-HPV plus Ipilimumab

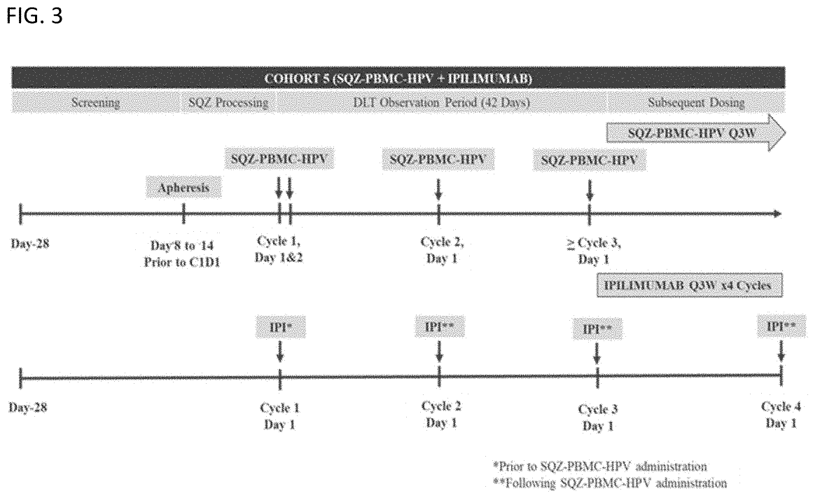

In Cycle 1, SQZ-PBMC-HPV is administered IV in accordance with the RP2D determined in Part 1; i.e., either as double priming on Days 1 and 2, or as a single-prime dose on Day 1. Ipilimumab, 3 mg/kg, is administered IV over 90 minutes, prior to SQZ PBMC HPV on Day 1. In Cycles 2, 3, and 4, ipilimumab is given on Day 1 following the administration of SQZ-PBMC-HPV. Ipilimumab is administered for a maximum of 4 cycles. SQZ-PBMC-HPV is given in 3-week cycles until discontinuation criteria are met or until the SQZ-PBMC-HPV supply has been exhausted, or for up to 1 year, whichever comes first (FIG. 3).

Cohort 6—SQZ-PBMC-HPV plus Nivolumab

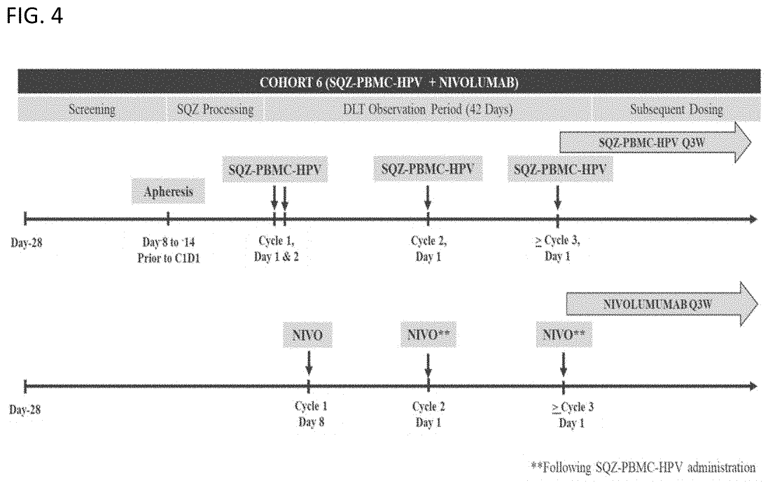

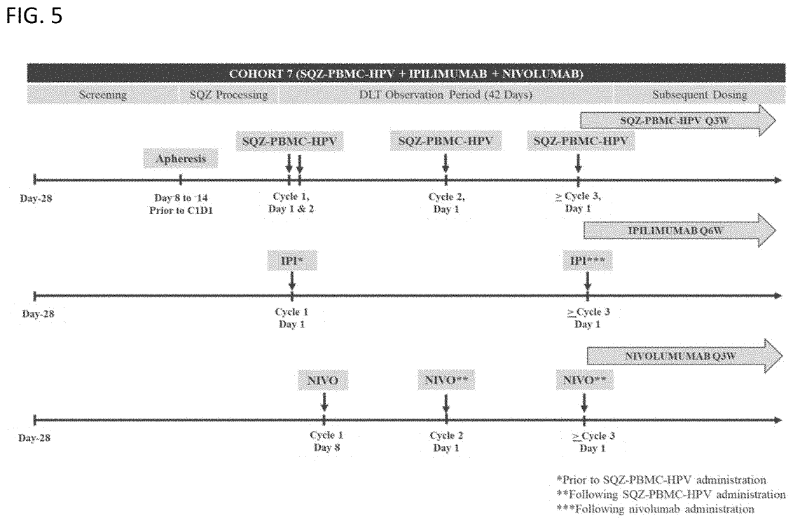

In Cycle 1, SQZ-PBMC-HPV is administered IV in accordance with the RP2D determined in Part 1; i.e., either as double priming on Days 1 and 2, or as a single-prime dose on Day 1. On Cycle 1 Day 8, nivolumab is administered at a dose of 360 mg IV, over 30 minutes. In subsequent cycles, SQZ-PBMC-HPV followed by nivolumab is administered on Day 1, every 3 weeks. Nivolumab may be given every 3 weeks for up to 2 years or until discontinuation criteria are met. SQZ-PBMC-HPV is administered in 3-week cycles until discontinuation criteria are met or the SQZ-PBMC-HPV supply has been exhausted, or for a maximum of 1 year, whichever comes first (FIG. 4).

Cohort 7—SQZ-PBMC-HPV plus Nivolumab plus Ipilimumab

In Cycle 1, SQZ-PBMC-HPV is administered IV in accordance with the RP2D determined in Part 1; i.e., either as double priming on Days 1 and 2, or as a single-prime dose on Day 1. Ipilimumab is administered IV at a dose of 1 mg/kg, over 30 minutes on Day 1, prior to SQZ-PBMC-HPV. On Cycle 1, Day 8, nivolumab 360 mg IV will be administered over 30 minutes. Nivolumab is given on Day 1 in subsequent, 3-week cycles, following administration of SQZ-PBMC-HPV. Ipilimumab is administered every 6 weeks, following administration of SQZ-PBMC-HPV and nivolumab in subsequent cycles. Nivolumab and ipilimumab may be given for 2 years from Cycle 1 Day 1 until 1 of the criteria for treatment discontinuation are met. SQZ-PBMC-HPV will be administered in 3-week cycles until discontinuation criteria are met or SQZ-PBMC-HPV supply has been exhausted, or for a maximum of 1 year, whichever comes first.

If, due to an immune-mediated AE, a patient meets criteria for discontinuation of checkpoint inhibitors, and the investigator is unable to determine whether the event is related to nivolumab or ipilimumab, the patient should discontinue both drugs, and may continue on SQZ-PBMC-HPV.

Cohort 7—SQZ-PBMC-HPV plus Nivolumab plus Ipilimumab

In Cycle 1, SQZ-PBMC-HPV is administered IV in accordance with the RP2D determined in Part 1; i.e., either as double priming on Days 1 and 2, or as a single-prime dose on Day 1. Ipilimumab is administered IV at a dose of 1 mg/kg, over 30 minutes on Day 1, prior to SQZ-PBMC-HPV. On Cycle 1, Day 8, nivolumab 360 mg IV is administered over 30 minutes. Nivolumab is given on Day 1 in subsequent, 3-week cycles, following administration of SQZ-PBMC-HPV. Ipilimumab is administered every 6 weeks, following administration of SQZ-PBMC-HPV and nivolumab in subsequent cycles. Nivolumab and ipilimumab may be given for 2 years from Cycle 1 Day 1 until 1 of the criteria for treatment discontinuation are met. SQZ-PBMC-HPV is administered in 3-week cycles until discontinuation criteria are met or SQZ-PBMC-HPV supply has been exhausted, or for a maximum of 1 year, whichever comes first.

If, due to an immune-mediated AE, a patient meets criteria for discontinuation of checkpoint inhibitors, and the investigator is unable to determine whether the event is related to nivolumab or ipilimumab, the patient should discontinue both drugs, and may continue on SQZ-PBMC-HPV.

For all cohorts in Part 2, the second SQZ-PBMC-HPV administration on Cycle 1 Day 2 is given during the ≥23-hour observation. Adverse events that develop after any administered dose resolved to ≤Grade 2 at time of subsequent administration. Similarly, AESIs that develop after any administered dose resolve to <Grade 2 at time of subsequent administration. If these retreatment criteria are met, the second SQZ PBMC HPV administration is given during the ≥23-hour observation period (i.e., between 16 and 24 hours post first dose). Patients are observed for a minimum of 4 hours after the second priming administration. The minimum interval between the 2 administrations is 16 hours. In each cohort, the first 2 patients must complete Cycle 1 Day 14 before additional patients in the cohort can be treated.

Patients are monitored for the occurrence of DLTs for 42 days after the first dose of SQZ PBMC-HPV in combination therapy cohorts.

In case of a DLT or other significant toxicity in individual patients, de-escalation to a lower SQZ PBMC-HPV dose occurs. Following review of the available safety, efficacy, and pharmacodynamic data from patients in individual combination safety cohorts, double priming may be determined as not advisable for 1 or more dose combinations. In this case, dropping the second (Cycle 1 Day 2) SQZ-PBMC-HPV dose may be recommended. Alternatively, a lower dose level for SQZ-PBMC-HPV may be explored (dose de-escalation). For instance, if DLT is observed in >33% of patients in individual combination safety cohorts, a cohort evaluating a lower SQZ-PTMC-HPV level is opened and explored.

### Part 3: Monotherapy Dose Expansion

Once the RP2D regimen is defined for SQZ PBMC HPV monotherapy, the Monotherapy Dose Expansion Phase is initiated. Patients are enrolled in disease-specific cohorts to further evaluate safety and tolerability as well as preliminary evidence of antitumor response. SQZ PBMC HPV is administered at the RP2D for monotherapy. Enrollment in Part 3 can occur in parallel with Part 2.

Cohort 8: locally advanced or metastatic HPV16+ head and neck cancer.

Cohort 9: locally advanced or metastatic HPV16+ cervical cancer.

Cohort 10: locally advanced or metastatic HPV16+ anal cancer.

Cohort 11: locally advanced or metastatic other HPV16+ cancer.

Cohorts 8, 9, 10, and 11 enroll in parallel. Each of the 4 dose expansion cohorts are enrolled using an optimal Simon 2-stage design. In the first stage, up to 10 patients are enrolled. If at least one response is observed in those 10 HIV negative patients, an additional 19 patients are enrolled, for a total of 29.

### Dosing Schedule and Study Duration

All patients undergo a single leukapheresis prior to the start of treatment. Patients undergo leukapheresis at the study sites; typically 8 to 14 days prior to the initial administration of SQZ PBMC HPV. Scheduling of the first administration of SQZ PBMC HPV takes into account site location and shipping logistics.

A cycle is defined as a treatment period of 21 days.

Patients receive SQZ PBMC HPV at 3-week intervals for up to 1 year, until investigational product is exhausted, or until treatment discontinuation criteria are met, whichever comes first.

Accumulating clinical evidence indicates some subjects treated with immune system stimulating agents may reveal signs of progression of disease (by conventional response criteria) before demonstrating clinical objective responses and/or stable disease. Two hypotheses have been put forth to explain this phenomenon. First, enhanced inflammation within tumors could lead to an increase in tumor size which would appear as enlarged index lesions and as newly visible small non-index lesions. Over time, both the malignant and inflammatory portions of the mass may then decrease leading to overt signs of clinical improvement (Wolchok et al, 2009). Alternatively, in some individuals, the kinetics of tumor growth may initially outpace anti-tumor immune activity. With sufficient time, the anti-tumor activity will dominate and become clinically apparent. Thus, it is important to assess RECIST 1.1 and iRECIST in parallel at each time point.

Patients may continue study therapy after initial RECIST 1.1 defined progression, and therefore allow for confirmation of disease progression according to iRECIST (Seymour et al, 2017) if the following criteria are met:

1. Investigator-assessed clinical benefit, and lack rapid disease progression

2. Tolerance of study drug as defined by the investigator

3. Stable performance status

4. Treatment beyond progression will not delay an imminent intervention to prevent serious consequence from rapidly progressing disease.

5. Lack of complications of disease progression (e.g., CNS metastases)

The assessment of clinical benefit takes into account whether the subject is clinically deteriorating and unlikely to receive further benefit from continued treatment.

The duration of treatment for the Monotherapy Dose Expansion Phase (Part 3) is dependent on the selected RP2D regimen.

SQZ PBMC-HPV is administered at 3 week intervals until treatment discontinuation criteria are met, or until investigational product is exhausted, or for a maximum of 1 year, whichever comes first. Treatment with the immune checkpoint inhibitor(s) in Cohorts 4, 6, and 7 may be continued for 2 years from Cycle 1 Day 1. Patients in Cohort 5 may complete 4 cycles of ipilimumab prior to exhausting their supply of SQZ-PBMC-HPV; in this case, patients may continue to receive single agent SQZ PBMC-HPV until treatment discontinuation criteria are met or until investigational product is exhausted, or for a maximum of 1 year, whichever comes first.

### Dose-Limiting Toxicity

A patient is considered evaluable for DLT assessment if he or she 1) experiences a DLT during the DLT assessment period, regardless of the cell dose received or 2) does not experience a DLT during the DLT assessment period after having received at least 70% of the intended dose of SQZ-PBMC-HPV during the DLT assessment period. Patients who do not experience a DLT yet received less than 70% of the intended SQZ-PBMC-HPV dose during the DLT assessment period are not considered evaluable for DLT and are replaced.

Patients experiencing a DLT that is not an IRR are discontinued from the study. If it is in the patient's best interest to continue treatment on investigational product, then the subsequent treatment will be determined by the Investigator in consultation with the Sponsor. For IRRs, the premedication or rate of administration is adjusted to enable the patient to remain on study.

A DLT is defined as an AE or abnormal laboratory value assessed by the Principal Investigator and confirmed by the SSC as unrelated to disease, disease progression, intercurrent illness, concomitant medications/procedures, or environmental factors, but related to SQZ-PBMC-HPV (either alone or in combination), occurring within either the first 28 days of treatment with monotherapy or the first 42 days of treatment with combination therapy, and which meets any of the pre-defined criteria as listed below using National Cancer Institute (NCI) Common Terminology Criteria for Adverse Events (CTCAE) version 5.0. Grading of CRS and neurotoxicity will use the American Society for Transplantation and Cellular Therapy (ASTCT) Consensus Grading, as referenced in Section 6.2.2 and Section 6.2.3, respectively.

Non-Hematologic Toxicity
Grade 4 or Grade 5.
Grade 3 toxicity that does not resolve to ≤Grade 1 or Baseline within 7 days despite optimal supportive care, except for Grade 3 CRS or neurotoxicity that does not resolve to ≤Grade 2 within 24 hours.
Grade 3 laboratory value that persists >7 days and requires medical intervention.
>Grade 3 hepatic toxicity lasting for >48 hours with the following exception: For patients with Grade 2 aspartate aminotransferase (AST), alanine aminotransferase (ALT), and/or alkaline phosphatase abnormalities at Baseline, only an increase to >8×ULN lasting >48 hours will be considered a DLT.
Liver tests abnormalities meeting Hy's law criteria.

Hematologic Toxicity
Any Grade 5 toxicity.
Any Grade 4 anemia.
Any ≥Grade 3 febrile neutropenia.
≥Grade 4 neutropenia (absolute neutrophil count<500/μL) lasting >7 days.
≥Grade 4 thrombocytopenia (<25,000/μL).
≥Grade 3 thrombocytopenia (<50,000/μL) lasting >7 days associated with clinically significant bleeding.

TEAEs at least possibly related to SQZ-PBMC-HPV (either alone or in combination) that result in permanent discontinuation or a delay of >14 days of Cycle 2 Day 1 of scheduled SQZ PBMC HPV administration.

Any other event that, in the judgement of the Investigator and Sponsor, is considered to be a DLT.

The following events are not considered a DLT:

Isolated Grade 3 lipase values that are not accompanied by ≥Grade 3 amylase values or clinical symptoms or radiographic evidence of pancreatitis.

Grade 3 CRS that improves to ≤Grade 2 within 24 hours with or without symptomatic treatment.

Grade 3 skin rash that resolves to ≤Grade 2 within 7 days with or without appropriate supportive care.

Immediate hypersensitivity reactions occurring within 2 hours of cell product administration that are reversible to <Grade 2 with 24 hours.

Grade 1 or Grade 2 electrolyte abnormalities that are corrected within 72 hours without clinical sequelae.

Alopecia.

A Grade 3 IRR that can be adequately managed with the addition of premedication or modification of the rate of administration will not be considered a DLT, unless these changes are considered applicable to all subsequent patients enrolled in the study. If the modification(s) applies to all subsequent patients, the cohort will restart for the DLT evaluation. The patient who experienced the Grade 3 infusion reaction may stay on study with modification to their premedication or the infusion rate.

If the MTD is not reached in any cohort, additional cell dose levels or regimens are tested. In the event of AEs covered by the definition of a DLT but unrelated to SQZ-PBMC-HPV, the findings will be discussed by the SSC.

Stopping Criteria for a Cohort and Stopping of Dose Escalation or Progression to Cohort and Termination of Study

The modified 3+3 rules define the ultimate decision to declare a cohort as safe. The minimum number of patients needed to confirm a cohort as safe is 3 patients with 0 DLTs, which can be increased up to 12 patients to confirm that a cohort is safe (i.e., <33% of patients with DLT; for instance, 6 patients with <2 DLTs, 9 patients with <3 DLTs, or 12 patients with <4 DLTs, whichever confirms the safety of the cohort). If none of the cohorts indicate that the MTD has been reached, additional cell dose levels or regimens may be tested. In the event of AEs covered by the definition of a DLT but unrelated to SQZ-PBMC-HPV.

An AE that meets the definition of a DLT and occurring outside the DLT window will not be counted as a DLT but instead will be considered in the overall safety assessment of a given cohort and the selection of an RP2D regimen.

The cohort stopping rule is the occurrence of >3 DLTs in up to 12 patients (˜33%) receiving investigational product within the same dose cohort. If the stopping rule is triggered, one of the following recommendations is made:

Declare the prior tolerated dose level as the MTD.

Declare a dose level the MAD level without observation of a DLT. Thus, the RP2D would not be the MTD.

Recommend testing of an intermediate dose level.

Recommend protocol amendment to increase patient safety.

Discontinue enrollment and/or the study.

Dosing of patients may be stopped in the interest of patient safety based on these general safety criteria:

Any SAE that is considered potentially life-threatening and is assessed by the Medical Monitor as related to investigational product.

Any other clinically significant change that indicates to the Investigator or Sponsor a major tolerability concern.

### Study Population

Patients who are HLA-A*02+ with advanced-stage HPV16+ solid tumors (head and neck, cervical cancer, and other tumor types). Additionally, HIV positivity is permitted for enrollment in the Combination Safety Phase. In the Escalation Phase, enrollment of HIV+ patients should be discussed with the Sponsor.

Patients may have received prior therapy with a PD-1, PD-L1, or CTLA-4 inhibitor (including ipilimumab or any other antibody or drug specifically targeting T cell co stimulation or checkpoint pathways).

### Number of Patients

The number of patients depends on safety and observed immunogenic effects. In the monotherapy Escalation Phase, it is anticipated that approximately 9 to 36 DLT-evaluable patients could be enrolled. If none of the planned cohorts indicate that the MTD has been reached, additional cell dose levels or regimens may be tested. Up to a total of 48 evaluable patients are enrolled in the Combination Safety Phase (n=12 per cohort). Up to a total of up to 116 patients are enrolled in the Expansion Phase (up to n=29 per cohort) if all cohorts are opened and pre-defined success criteria allow to open the second stage of each cohort. Depending on the need to replace patients within cohorts, it is expected that approximately 173 to 200 evaluable patients will be treated in the study. If none of the planned cohorts indicate that the MTD has been reached, additional cell dose levels or regimens may be tested.

### Inclusion Criteria

1. Male or female patients≥18 years of age who are HLA-A*02+, as confirmed by genotyping assay from blood.
2. Histologically confirmed incurable or metastatic solid tumors (including but not limited to cervical and head and neck tumors) that are HPV16+.
3. For cervical cancer, which is not amenable to curative treatment with surgery, radiation, and/or chemoradiation therapy, the cancer must have progressed after prior systemic chemotherapeutic treatment with a platinum-based regimen in the adjuvant or recurrent setting. Patients must have progressive disease while receiving or after the completion of the most recent prior treatment.
For patients who are intolerant to or refuse a platinum-based systemic chemotherapeutic treatment for recurrent disease, reasons must be documented.
4. For recurrent and metastatic head and neck cancer, which is not amenable to curative treatment with surgery, radiation, and/or chemoradiation therapy, the cancer must have progressed following at least 1 prior platinum-based chemotherapy in the primary, adjuvant or recurrent setting and been offered checkpoint immunotherapy. Patients who relapsed after platinum-containing definitive chemoradiation or after adjuvant chemoradiation are eligible if a platinum re-challenge at time of relapse is not seen as beneficial.
For patients who are intolerant to or refuse a platinum-based systemic chemotherapeutic treatment for recurrent disease, reasons must be documented.
5. Patients with incurable or metastatic HPV16+ cancers other than cervical or head and neck cancer must have progressed after at least 1 available standard therapy for incurable disease, or the patient is intolerant to or refuses standard therapy(ies) or has a tumor for which no standard therapy(ies) exist.

### Escalation Phase (Part 1) and Combination Safety Phase (Part 2)

a. Enrollment of HIV+ patients should be discussed with the Sponsor.

### Expansion Phase (Part 3)

b. HIV+ patients should be discussed with the Sponsor to ensure that a ratio of 1 out of 6, or a multiple, per cohort is not exceeded. HIV+ patients must have received at least 1 prior systemic cancer therapy or not qualify for standard of care. HIV+ patients must meet the following criteria to be eligible:
CD4+ T-cell count>350 cells/mL and no history of acquired immunodeficiency syndrome (AIDS)-defining opportunistic infections within past 12 months Enrollment of patients on prophylactic antimicrobials should be discussed with the Sponsor.
6. Eastern Cooperative Oncology Group (ECOG) performance status (PS) of 0 to 1.
7. Patients must agree to venous access for the leukapheresis and be willing to have a central line inserted if venous access is an issue.
8. Patients with unresectable or metastatic solid tumors must have a lesion that can be biopsied with acceptable clinical risk and agree to have a fresh biopsy at Screening and on Cycle 2 Day 8 (±2 days).
   a. A lesion in a previously irradiated area could be biopsied as long as there is objective evidence of progression of the lesion before study enrollment.
9. At least 1 measurable lesion according to RECIST 1.1.
   a. A lesion in a previously irradiated area is eligible to be considered as measurable disease if there is objective evidence of progression of the lesion before study enrollment.
10. Adequate organ function and bone marrow reserve as indicated by the following laboratory assessments performed within 14 days prior to the leukapheresis:
   a. Bone marrow function: absolute neutrophil count≥1000/4; hemoglobin≤9 g/dL; platelet count≥75,000/4. NOTE: In stabilized patients with hemoglobin values<9 g/dL, a blood transfusion may be utilized to meet inclusion criterion.
   b. Hepatic function: total serum bilirubin≤1.5×ULN; serum AST/ALT, ≤2.5×ULN (≤5×ULN in the presence of hepatic metastases); alkaline phosphatase<2.5×ULN with the following exception: patients with liver and bone involvement: alkaline phosphatase≤5×ULN.
      i. Patients with inherited disorders of bilirubin metabolism should be discussed with the Sponsor.
   c. Renal function: serum creatinine≤2.5×ULN or creatinine clearance≥30 mL/min based either on urine collection or Cockcroft-Gault estimation.
   d. Coagulation profile: prothrombin time (PT), international normalized ratio (INR)/partial thromboplastin time (PTT)≤1.5×ULN. Patients on a stable, maintenance regimen of anticoagulant therapy for at least 30 days prior to leukapheresis may have PT/INR measurements>1.5×ULN if, in the opinion of the Investigator, the patient is suitable for the study. An adequate rationale must be provided to the Sponsor prior to enrollment.
11. Patients with immune-mediated endocrinopathies following treatment with immune checkpoint inhibitors requiring hormone replacement therapy are eligible.
   a. Patients requiring prednisone as part of hormone replacement therapy are eligible if the daily doses do not exceed 10 mg.
12. Female patients of childbearing potential must:
   a. Have a negative serum beta human chorionic gonadotropin (β-hCG) pregnancy test at Screening, and
   b. Agree to use double contraception from the time of informed consent until at least 5 months after the last dose of immune checkpoint inhibitor or SQZ PBMC-HPV.
13. Male patients who are not vasectomized must be willing to use condoms from the time of informed consent until at least 5 months after the last dose of immune checkpoint inhibitor or SQZ PBMC HPV.
14. The patient is capable of understanding and complying with the protocol and has signed the required informed consent form (ICF). The appropriate ICF must be signed before relevant study procedures are performed. If applicable, the female partner of a male patient understands and signs the pregnant partner ICF.

### Exclusion Criteria

1. Treatment with anticancer therapy, including investigational therapy, within 2 weeks prior to leukapheresis. For prior therapies with a half-life longer than 3 days, timing of discontinuation of the therapy should be discussed with the Sponsor.
2. Patients with >Grade 1 AEs (except Grade 2 alopecia) according to NCI CTCAE version 5.0 related to previous treatment with anticancer or investigational therapy that do not resolve (i.e., ≤Grade 1 or better) at least 2 weeks prior to leukapheresis.
3. History of any Grade 4 irAE from prior immunotherapy (patients with endocrinopathy managed with replacement therapy or asymptomatic elevation of serum amylase or lipase are eligible), any irAE that led to permanent discontinuation of prior immunotherapy, or any Grade 3 irAE that occurred ≤6 months prior to leukapheresis.
4. Patients treated with non-corticosteroid based immunosuppressive agents within the last 6 months may not be eligible and should be discussed with the Sponsor.
5. Patients with active, known, or suspected autoimmune disease may not be eligible and should be discussed with the Sponsor.
6. Patients with prior allogeneic bone marrow or solid organ transplantation may not be eligible and should be discussed with the Sponsor.
7. Live virus vaccination within 4 weeks prior to leukapheresis.
8. Systemic treatment with either corticosteroids (>10 mg of prednisone or the equivalent per day) or other immunosuppressive medications within 14 days prior to leukapheresis. Inhaled, intranasal, intra-articular and topical (including ocular) steroids are allowed. The use of steroid replacement for patients with adrenal insufficiency is allowed. The use of fludrocortisone for mineralocorticoid replacement in patients with adrenal insufficiency is allowed.
9. Has known active central nervous system metastases and/or carcinomatous meningitis. Patients with previously treated brain metastases may participate provided they are stable (without evidence of progression by imaging for at least 4 weeks prior to the first dose of investigational product and any neurologic symptoms have returned to Baseline), have no evidence of new or enlarging brain metastases, and are not using steroids for at least 7 days prior to leukapheresis. This exception does not include carcinomatous meningitis, which is excluded regardless of clinical status.
10. History of interstitial lung disease requiring steroids, idiopathic pulmonary fibrosis, pneumonitis (including drug induced), or organizing pneumonia (e.g., bronchiolitis obliterans, cryptogenic organizing pneumonia).
   a. Patients with asymptomatic pneumonitis who have not required steroid therapy for pneumonitis are eligible.
11. Clinically significant cardiac disease, including unstable angina, acute myocardial infarction within 6 months prior to leukapheresis, New York Heart Association class III or IV congestive heart failure, and arrhythmia requiring therapy.
12. Systemic arterial thrombotic or embolic events, such as cerebrovascular accident (including ischemic attacks) within 1 month prior to leukapheresis.
13. Systemic venous thrombotic events (e.g., deep vein thrombosis) or pulmonary arterial events (e.g., pulmonary embolism) within 1 month prior to leukapheresis.
   a. Patients with venous thrombotic events before leukapheresis on stable anticoagulation therapy are eligible.
14. History or presence of an abnormal electrocardiogram (ECG) that, in the Investigator's opinion, is clinically meaningful.
15. Left ventricular ejection fraction (LVEF)<50%.
16. Major surgery within 2 weeks of leukapheresis; following major surgeries >2 weeks prior to leukapheresis, all surgical wounds must be healed and free of infection or dehiscence.
17. Any other clinically significant comorbidities, such as active infection, known psychiatric or neurological disorder, or any other condition, which in the judgment of the Investigator, could compromise compliance with the protocol, interfere with the interpretation of study results, or predispose the patient to safety risks.
18. Known active hepatitis B or hepatitis C, or active Mycobacterium tuberculosis infection.
19. Patient has history of alcohol and/or illicit drug abuse within 12 months of entry.
20. Female patients who are breastfeeding or have a positive serum pregnancy test at the Screening visit or enrollment.
21. Patient has a history of allergy or hypersensitivity to any component of SQZ PBMC HPV.
22. History of severe allergic anaphylactic reactions to chimeric, human, or humanized antibodies or infusion proteins (combination cohorts only).
23. Known hypersensitivity to atezolizumab, ipilimumab, nivolumab, Chinese hamster ovary cell products or any component of the atezolizumab, ipilimumab, or nivolumab formulation (combination cohorts only).

### Leukapheresis

The goal of the leukapheresis is to provide a yield of WBCs for each patient of approximately 10 to 14×10⁹ cells to support extended treatment duration. Efforts are made to adjust the procedure in case a low yield is expected, e.g., by processing a blood volume of up to 15 liters. In accordance with local procedures, if possible, a WBC or complete blood cell count is taken during leukapheresis so that the processed blood volume may be increased. In the event a WBC or complete blood cell count cannot be taken during leukapheresis, a sample is taken at the end of leukapheresis, if possible, to determine the WBC count in the leukopak. The results are processed as soon as possible and provided to the Sponsor in real-time.

### Tumor Response Assessment and Schedule

Tumor assessment is performed at Screening (baseline) and tumor response is assessed by the Investigator every 9 weeks (±7 days) for 1 year after the first dose of SQZ PBMC HPV, then every 12 weeks (±7 days) thereafter until disease progression as confirmed by RECIST and iRECIST, unacceptable toxicity, withdrawal of consent, death or 2 years from the date of the first administration of SQZ PBMC HPV, whichever occurs first.

Disease is evaluated via radiographic imaging. Patients who experience disease progression per RECIST 1.1 may continue dosing if considered in their best interest by the treating Investigator to allow for confirmation of disease progression; i.e., iCPD according to iRECIST (Seymour et al, 2017).

If a patient discontinues investigational product for reasons other than progression, that patient should continue to be imaged following the schedule outlined above. Radiographic assessments should be obtained and recorded in the CRF.

At Screening and all subsequent time points, cervical, anal/rectal, vulvar/vaginal, and penile carcinomas require computed tomography (CT) of the torso (chest, abdomen and pelvis) and all known sites of disease; oropharyngeal carcinomas require CT of head, neck, and chest and other areas of known involvement. If, for justifiable reasons, CT scans cannot be used or do not allow for an appropriate tumor assessment, magnetic resonance imaging (MRI) is permitted and the Sponsor should be informed during Screening. The same radiographic procedure used to assess disease sites at Screening are used throughout the study. For all other advanced solid tumor types, the Investigator images all known sites of disease using the imaging modality the Investigator believes best for that tumor type.

Magnetic resonance imaging of the brain is required at Screening in all patients with a history of brain metastases, and may be repeated at subsequent time points in any patient with a history of brain metastases and/or in any patient who develops symptoms suggestive of brain metastasis. If a patient is unable to tolerate or has a contraindication for MRI, CT scan can be used.

The same evaluator performs assessments to ensure internal consistency across visits. At the Investigator's discretion, CT scans should be repeated at any time if PD is suspected. For patients who achieve a partial response (PR) or complete response (CR), tumor assessment should be repeated 4 weeks later to confirm response.

### Pharmacodynamic Assessments, Including Immunogenic Measurements

### Sample Collection Schedule

### Tumor Biopsies

Prior to leukapheresis, patients undergo a Screening tumor biopsy (primary tumor or metastasis) that can be from a previously radiated site with active tumor growth. All patients are required to undergo a repeat tumor biopsy of the same primary tumor or metastasis on Cycle 2 Day 8 (±2 days). If possible, an additional repeat tumor biopsy is obtained (predose) at Cycle 5 Day 1 (+2 days); this sample is optional. If preliminary data suggest that modification of the on treatment tumor biopsy time point would be more appropriate, alternative on-treatment tumor biopsy time points may be considered.

The fresh tumor biopsy taken at Screening should be from the primary tumor or metastasis site and subsequent biopsies should be from the same primary tumor or metastasis biopsied at Screening.

### Pharmacodynamic Assessments

Whenever possible, Baseline samples will be used for longitudinal assessment of cellular correlative tests, including, but not limited to, immunophenotyping by flow cytometry including tetramer staining, assessment of T cell production of cytokines following co-culture with HPV peptides (IFNγ and Granzyme B enzyme-linked immunoSpot [ELISPOT]), and circulating cell free HPV16 DNA (cfHPV DNA). Baseline tumor biopsies and selected blood samples will be used for comparison to post-treatment samples only (Table 2).

TABLE 2
Pharmacodynamic and Immunogenic Assessments

| Sample Source | Assay |
|---|---|
| Blood | IFNγ and GranzymeB ELISPOT (with and without in |
| | vitro stimulation) |
| | E6, E7 tetramer staining (combine with surface marker |
| | staining) |
| | Circulating cell-free HPV16 DNA (cfHPV DNA) levels |
| Tumor tissue | Immunohistochemistry assessment of changes in tumor |
| | micro-environment |

Abbreviations:
DNA = deoxynucleic acid;
HPV16 = human papilloma virus strain 16;
IFN [symbol] = interferon gamma

The information about endogenous immune responses detected via ELISPOT will inform the immunohistochemical analysis of tumor biopsies.

### Cytokine Assessments

All patients have blood samples collected for cytokines. Patients with Grade 2, 3, or 4 CRS have additional cytokine plasma levels performed during Grade 2, 3, or 4 CRS events. Blood collections are obtained at time of diagnosis of a CRS, at time of an increase in severity (e.g., when a Grade 2 CRS progresses to a Grade 3 CRS), onset of neurological symptoms, and at time of discharge or resolution.

The evaluation of a cytokine panel includes, but is not limited to, IFN gamma (IFN?) and IL 6. Although CRS may have a delayed onset, it rarely presents beyond 14 days after initiation of therapy. Patients exhibiting symptoms consistent with CRS presenting outside this window are carefully evaluated for other causes.

Cytokines are monitored for pharmacodynamic assessments. Baseline and post treatment serum samples are collected to assess anti-tumor immune responses by measuring cytokines that could provide information about drug inflammatory responses.

### Safety Assessments

Safety is evaluated in this study through the monitoring of all SAEs and nonserious AEs and laboratory abnormalities, defined and graded according to NCI CTCAE version 5.0. General safety assessments include physical examinations and specific laboratory evaluations, including serum chemistry, coagulation, and blood counts including differential. SAEs and ≥Grade 2 AESIs will be reported in an expedited fashion for entry into the safety database.

During the conduct of the study, the totality of safety events observed is reviewed (including CRS events that resolved to Grade 2) and a decision is made if a given event requires initiation of staggered enrollment of patients following this event. Staggered enrollment in potential additional monotherapy cohorts (Part 1) or in the Combination Safety Cohorts (Part 2) require all subsequent newly enrolled patients in a cohort or cohorts to be staggered by 1 week. Semi sequential enrollment of patients may continue in some cohorts, if applicable. Patients will have cytokine release assays performed during the study. Patients with Grade 2, 3, or 4 CRS have additional blood samples taken for safety laboratories and the evaluation of the cytokine panel.

Exposure to immune checkpoint inhibitors may increase the risk of irAEs, specifically autoimmune conditions. As such, irAEs are recognized early and treated promptly to avoid potential major complications.

All patients return to the clinic for a Safety Follow-up visit within 15 to 45 days after the last dose of investigational product. All AEs and SAEs are recorded until 6 weeks after last dose of investigational product (EOD6W) or up to 45 days from drop out or until initiation of another anticancer therapy, whichever occurs first. Only ongoing SAEs determined by the Investigator to be possibly, probably, or definitely related to SQZ-PBMC-HPV monotherapy or combination therapy are followed up.

### Physical Examination and Height and Weight

A physical examination includes height (Screening only), weight, and an assessment of general appearance and an evaluation of the following systems: dermatologic, head, eyes, ears, nose, mouth/throat/neck, thyroid, lymph nodes, respiratory, cardiovascular, gastrointestinal, extremities, musculoskeletal, neurologic, and gynecologic and genitourinary systems, as indicated. It is especially important to capture weight during the physical examination of the patient within 24 hours of leukapheresis, as patient dosing is determined by weight.

### Performance Status

Eastern Cooperative Oncology Group scales and criteria are used to assess a patient's performance status, assess how the disease affects the daily living abilities of the patient, and determine appropriate treatment and prognosis.

### Vital Signs

Vital signs are collected and include measurement of systolic and diastolic blood pressure while the patient is in a seated position, heart rate, temperature, and respiratory rate. 12-Lead Electrocardiograms

12-lead ECGs are performed by qualified site personnel using an ECG machine that determines heart rate, PR interval, QRS interval, RR interval, QT interval, and QTc interval collected by QTcB (QTc corrected by Bazett's formula) and QTcF (QTc corrected by Fridericia's formula). During the collection of ECGs, patients are in a resting position, in a quiet setting without distractions (e.g., television, cell phones) for at least 10 minutes before ECG collection.

All ECGs must be evaluated by a qualified physician for the presence of abnormalities. Echocardiograms

Echocardiogram or multigated acquisition (MUGA) scans are performed to measure LVEF at Screening and as clinically indicated.

### Laboratory Assessments

Samples for clinical laboratory assessments will be collected. Clinical laboratory tests outlined in Table 3 are performed by the site. Samples for laboratory tests outlined in Table 3 are collected in appropriate tubes and handled according to standard procedures of the site.

Clinical laboratory variables are listed in Table 3.

TABLE 3
Clinical Laboratory Assessments
Hematology^{a}: Required at all visits.

| | |
|---|---|
| Total white blood cell count | Neutrophils (percentage and absolute count) |
| Red blood cell count | Lymphocytes (percentage and absolute count) |
| Hemoglobin | Monocytes (percentage and absolute count) |
| Hematocrit | Eosinophils (percentage and absolute count) |
| Mean corpuscular volume | Basophils (percentage and absolute count) |
| Mean corpuscular | Platelet count |
| hemoglobin | |
| Mean corpuscular | Red blood cell distribution width |
| hemoglobin concentration | |

Coagulation^{a,b,c}: Required at all visits unless otherwise specified.

| | |
|---|---|
| Prothrombin time (PT) | International normalized ratio (INR) |
| Partial thromboplastin time | D-dimer^{c} |
| (PTT) | |
| Fibrinogen^{c} | von Willebrand factor^{c} |

Clinical Chemistry: Required at all visits except leukapheresis.

| | |
|---|---|
| Alanine aminotransferase | Gamma glutamyl transferase |
| (ALT) | |
| Albumin | Glucose |
| Alkaline phosphatase | Lactate dehydrogenase |
| Aspartate aminotransferase | Phosphorus |
| (AST) | |
| Blood urea nitrogen | Potassium |
| Calcium | Sodium |
| Chloride | Thyroid function test (TSH, free |
| | T3, and free T4) |
| Cholesterol | Total bilirubin |
| Creatinine | Total protein |
| C-reactive protein | Triglycerides |
| Creatine kinase | Uric acid |
| Ferritin | Magnesium |

Urinalysis: Required at all visits except leukapheresis.

| | |
|---|---|
| Bilirubin | Blood |
| Glucose | pH and specific gravity |
| Ketones | Protein |
| Leukocytes | Urobilinogen |
| Nitrite | Leukocyte esterase |

Microscopic (if macroscopic panel is abnormal) white blood cells, RBC,
casts, crystals, bacteria, and epithelial cells
Viral Serology: Required at Screening and as clinically indicated.

| | |
|---|---|
| Hepatitis B core antibody | Human immunodeficiency virus (HIV) |
| (anti-HBc) | |
| IgM antibody to anti-HBc | (Types 1 and 2) antibodies |
| (IgM anti-HBc) | |
| Hepatitis B surface antigen | Hepatitis C virus antibody (anti-HCV) |
| (HBsAg) | |

Pregnancy Testing: Required at Screening and 6 weeks after the last
administration of investigational product.
Serum human beta chorionic gonadotrophin (women of childbearing
potential only) required at screening; urine β-hCG may be used in
subsequent assessments.
^{a}Results for these laboratory tests are required to be collected prior to, or the day of, leukapheresis, with the results available prior to leukapheresis.
^{b}Results of coagulation parameters are required on the day of, or the day following any tumor biopsy.
^{c}Collected at baseline and in cases of CRS.
Abbreviations:
CRS = cytokine-release syndrome;
T3 = triiodothyronine;
T4 = thyroxine;
TSH = thyroid-stimulating hormone

### Adverse Events

### Definitions

### Adverse Event

An AE is any untoward medical occurrence in a patient that does not necessarily have a causal relationship with the investigational product administered. An AE can therefore be any unfavorable or unintended sign (including an abnormal laboratory finding), symptom, or disease temporally associated with the use of an investigational product, whether or not related to the investigational product. Adverse events may be new events or may be pre-existing conditions that have become aggravated or have worsened in severity or frequency.

Adverse events may be clinically significant changes from Baseline in physical examination, laboratory tests, or other diagnostic investigation.

In this study, an AE is treatment-emergent if the onset time is after administration of investigational product through 6 weeks after the last dose of investigational product.

### Serious Adverse Event

An SAE is any AE that results in any of the following:
Death.
Is immediately life-threatening.
Requires in-patient hospitalization or prolongation of existing hospitalization.
Results in persistent or significant disability or incapacity.
Results in a congenital abnormality or birth defect.
Is an important medical event that may jeopardize the patient or may require medical intervention to prevent 1 of the outcomes listed above.

All SAEs that occur after any patient has signed the ICF, before treatment, during treatment, or within 30 days following the cessation of treatment, whether or not they are related to the study, must be recorded on the appropriate clinical procedure form.

### Adverse Events of Special Interest

An AESI is an AE (serious or nonserious) of scientific and medical concern specific to investigational product, for which ongoing monitoring and immediate notification by the Investigator to the Sponsor is required. Such AEs may require further investigation to characterize and understand them. Adverse events of special interest may be added or removed during the study by a protocol amendment.

The following AEs are considered AESIs:
Events suggestive of hypersensitivity, cytokine release, systemic inflammatory response syndrome, systemic inflammatory activation.
Influenza-like illness.
Infusion-reaction syndrome.
irAEs related to immune therapy, such as myocarditis, neurological irAEs, transaminitis of immune-related etiology, and nephritis.

In addition, the following events are reported to the Sponsor:
A suspected overdose of SQZ-PBMC-HPV.
Liver tests abnormalities meeting Hy's law criteria, i.e., an AST or ALT laboratory value≥3×ULN and a total bilirubin laboratory value≥2×ULN and, at the same time, an alkaline phosphatase laboratory value<2×ULN, as determined by protocol specified or unscheduled laboratory testing.

### Assessment of Severity

The NCI CTCAE version 5.0 is used to assess and grade severity for AEs and for laboratory abnormalities. ASTCT Consensus Grading will be used for CRS and ICANS. Each AE term will be mapped to the latest version of Medical Dictionary for Regulatory Activities (MedDRA) term and code.

If the event is not covered in CTCAE version 5.0, the guidelines shown in Table 4 should be used to assess severity.

TABLE 4
Severity and Toxicity Grade of Events Not Covered by CTCAE

| Toxicity | | |
|---|---|---|
| Grade | Severity | Description |
| Grade 1 | Mild | Transient or mild discomfort (<48 hours); no |
| | | medical intervention/therapy required. |
| Grade 2 | Moderate | Mild to moderate limitation in activity - some |
| | | assistance may be needed; no or minimal medical |
| | | intervention/therapy required. |
| Grade 3 | Severe | Marked limitation in activity, some assistance |
| | | usually required; medical intervention/therapy |
| | | required, hospitalization possible. |
| Grade 4 | Life- | Extreme limitation in activity, significant |
| | threatening | assistance required; significant medical |
| | | intervention/therapy required, hospitalization |
| | | or hospice care possible. |
| Grade 5 | Fatal | The patient died due to the event. |

Source: (NIAID, 2003)
Abbreviations:
CTCAE = Common Terminology Criteria for Adverse Events

### Assessment of Causality

Relationship to investigational product is assessed by the Investigator. Accordingly, the AE and SAE report forms includes the option to attribute causality to SQZ-PBMC-HPV, atezolizumab, ipilimumab, nivolumab, or a combination. For patients receiving combination therapy with SQZ PBMC-HPV and immune checkpoint inhibitor(s), causality is assessed individually for each protocol-specified therapy. A reasonable suspected causal relationship is attributed to the immune checkpoint inhibitor alone if the event is consistent with the immune checkpoint inhibitor labeling.

The relationship of the AE to investigational product (i.e., SQZ-PBMC-HPV, atezolizumab, ipilimumab, nivolumab, or a combination) is documented as follows:

Definite: The AE is clearly related to the investigational product.

Probable: The AE is likely related to the investigational product.

Possible: The AE may be related to the investigational product.

Unlikely: The AE is doubtfully related to the investigational product.

Unrelated: The AE is clearly NOT related to the investigational product.

An Investigator who is qualified in medicine makes the determination of the relationship to the investigational product for each AE. The Investigator decides whether, in his or her medical judgment, there is a reasonable possibility that the event may have been caused by the investigational product. If no valid reason exists for suggesting a relationship, then the AE is classified as “unrelated.” If there is any valid reason, even if undetermined, for suspecting a possible cause-and-effect relationship between the investigational product and the occurrence of the AE, then the AE is considered “related.”

If the relationship between the AE/SAE and the investigational product is determined to be “definite,” possible,” or “probable” the event is considered related to the investigational product for the purposes of expedited regulatory reporting.

### Expectedness

An AE that is not listed in, or is inconsistent with the specificity or severity, from the applicable product information (e.g., the IB for SQZ-PBMC-HPV or the approved labeling for atezolizumab, ipilimumab, or nivolumab) is considered unexpected.

### Efficacy Analyses

### Definitions

Progression-free Survival (PFS) is defined as the time from Cycle 1 Day 1 to first documentation of objective tumor progression (PD, radiological) according to RECIST 1.1 or death due to any cause, whichever comes first. Progression-free survival data will be censored on the date of last tumor assessment documenting absence of PD for patients who do not have objective tumor progression and are still on study at the time of the analysis, are given antitumor treatment other than investigational product, or are removed from treatment follow-up prior to documentation of objective tumor progression. Patients having no tumor assessments after enrollment who are not known to have died will have PFS censored on Cycle 1 Day 1. PFS will be assessed by both RECIST 1.1 and iRECIST criteria to accommodate different practice across participating sites.

Overall Survival (OS) is defined as the time from the date of Cycle 1 Day 1 to date of death due to any cause. In the absence of confirmation of death, survival time will be censored at the last date the patient is known to be alive. Patients lacking data beyond Cycle 1 Day 1 will have their survival times censored on Cycle 1 Day 1.

Objective Response Rate (ORR) is defined as the proportion of patients with CR or PR according to RECIST 1.1. Objective response rate will be provided as unconfirmed and confirmed ORR. Confirmed responses are those that persist on repeat imaging study at least 28 days after the initial documentation of response. Similarly, iORR by iRECIST will also be summarized and reported.

Duration of Response (DoR) is defined as the time from the first documentation of PR or CR to the first documentation of objective tumor progression or death due to any cause. Duration of response data will be censored on the day of the last tumor assessment documenting absence of PD for patients who do not have tumor progression and are still on the study at the time of an analysis, are given antitumor treatment other than the investigational product, or are removed from the study follow-up prior to documentation of objective tumor progression will be censored at the last tumor assessment. Similarly, iDoR by iRECIST will also be summarized and reported.

Best Overall Response (BOR) is determined once all tumor assessments from Cycle 1 Day 1 until disease progression or death are recorded. In general, it is the best response across all assessments; however, confirmation of CR, PR, and stable disease (SD) will also be used in BOR determination. To confirm CR or PR, changes in tumor measurements must be confirmed by repeat assessments that should be no less than 4 weeks (28 days) after the criteria for response are first met. To confirm SD, it must have occurred at least 12 weeks from Cycle 1 Day 1; otherwise, BOR will depend on subsequent assessments. Best overall response will be summarized by percentages and as a time to event variable for time to best response using enrollment as the anchor date. Similarly, iBOR by iRECIST will also be summarized and reported.

Disease Control Rate (DCR) is the proportion of patients in whom the BOR is determined as CR, PR, or SD by RECIST 1.1 at defined time points. All patients in the safety population with measurable disease at Baseline and eligible for tumor assessment will be considered as the denominator of the DCR proportion at 3, 6, and 12 months. Similarly, iDCR by iRECIST will also be summarized and reported.

Stable Disease 12 weeks is the proportion of patients for whom the BOR is determined as CR, PR, or SD by RECIST 1.1 and is maintained for at least 12 week. All patients in the safety population with measurable disease at Baseline and eligible for tumor assessment will be considered as the denominator of the proportion at 3, 6, and 12 months. Similarly, SD for 12 weeks by iRECIST will also be summarized and reported.

### Analyses

Efficacy analyses is performed on the safety population. Antitumor activity (ORR, PFS, OS) is described for patients with documented HLA class I expression as well. If the Per Protocol population differs from the Safety Population, efficacy analyses will be also performed using the PP population.

The Kaplan-Meier method is used to estimate the median PFS and 2-sided 95% confidence interval. Patients who die, regardless of cause of death, are considered to have had an event unless subsequent anticancer therapy was received prior to death. If subsequent therapy is received, the patient will be censored of date of last evaluable tumor assessment prior to subsequent therapy. Patients who withdraw consent for the study are considered censored at the time of the last evaluable tumor assessment prior to withdrawing consent. Patients who are still alive at the time of the clinical data cut-off date are censored at the most recent evaluable tumor assessment. All patients who were lost to follow-up prior to the clinical data cut-off date will also be considered censored at the time of the last evaluable tumor assessment prior to lost to follow up.

Duration of response, time to best overall response, and overall survival will use the same method as PFS. In addition, iPFS, iBOR, iDCR, and time to iBOR using iRECIST are analyzed and reported using similar methods.

Objective Response Rate (ORR) are presented as a proportion with a 95% confidence interval based on the exact binomial distribution. The point estimate and 2-sided 95% confidence interval of ORR will be provided. DCR and SD lasting at least 12 weeks will be reported as point estimates.

### Safety Analyses

All safety parameters are analyzed using the Safety population. Safety parameters include: AEs, laboratory evaluations, vital signs, ECOG, exposure, ECG, ECHO/MUGA and physical examinations.

The primary endpoint for safety is the number of patients with any AE and observed toxicity to SQZ-PBMC-HPV administration, where the severity is assessed using NCI CTCAE version 5.0. All AEs with onset after the first administration of SQZ-PBMC-HPV are included in the analysis. Adverse events are collected beginning at signing informed consent; however analyses is performed focusing on treatment-emergent AEs.

The AEs is analyzed using descriptive statistics. For patients with multiple incidences of a given AE, the highest severity is used.

### Adverse Events

The AEs are coded using the current version of the MedDRA coding dictionary.

An AE is treatment-emergent if the onset occurs on Cycle 1 Day 1 through 6 weeks after the last dose of investigational product. For AEs with partial onset times, non-missing date parts are used to determine if the AE is treatment-emergent. If a determination cannot be made as to when the AE occurred relative to investigational product administration, the AE are classified as treatment-emergent. Treatment-emergent AEs also include any AEs that were present prior to the first administration of investigational product and worsened in toxicity after the administration.

The analyses described in this section is based on TEAEs, plainly referred to as AEs in this section for brevity.

Adverse events considered as possibly, probably, or definitely related to investigational product by the Investigator is classified as related for summary purposes.

The number and percentage of patients with any AE, any related AE, any SAE, any related SAE, any Grade 3 or higher AE, any related Grade 3 or higher AE, as well as the total number of events for each category, is summarized. The number of deaths due to an AE, hospitalization due to an AE, and treatment discontinuation due to an AE, as well as DLTs and AESIs, are summarized.

The number and percentage of patients with an AE, as well as the total number of AEs, are summarized by system organ class and preferred term. This tabulation is repeated for related AEs, AESIs, SAEs, related SAEs, and ≥Grade 3 AEs, and related ≥Grade 3 AEs.

All AEs, including non-TEAEs, are provided in patient listings. Patient listings of AEs causing discontinuation of investigational product, AEs leading to death, SAEs, related AEs, AESI, DLTs, and severe AEs are produced.

### Clinical Laboratory Evaluation

Baseline is defined as the last non-missing value prior to the first exposure to investigational product. This is typically Cycle Day 1 pre-dose, but may be earlier. Actual values and changes from Baseline clinical laboratory tests are summarized by study visit.

Laboratory test results are classified according to NCI CTCAE version 5.0 and clinical significance as determined by the Investigator. If more than 1 laboratory result is reported per study visit per parameter, the result yielding the most severe classification is selected for analysis. Shift tables are created to show the greatest change from baseline for graded laboratory parameters.

All laboratory assessments are provided in listings.

Patients with clinically significant abnormal laboratory test results are listed. This listing includes all results of the laboratory parameter that was abnormal and determined to be clinically significant by the Investigator for a patient across study visit.

### Vital Signs

Baseline is defined as the last non missing value prior to the first exposure to investigational product. Actual values and changes from Baseline in vital signs are summarized by study visit and study time point. All vital sign data are presented in patient listings.

Vital sign values are classified according to the clinical significance as determined by the Investigator. The number of patients with a non-missing result, the number and percentage of patients with a non-clinically significant result, and clinically significant result is summarized by study visit and study time point. If more than 1 vital sign result is reported per study visit and study time point per parameter, the result yielding the most severe classification is selected for analysis.

Patients with clinically significant vital sign values are listed. This listing includes all results of the vital sign parameter that was determined by the Investigator to be clinically significant for a patient across study time points.

### Physical Examination

Abnormal physical examination findings are listed.

### 12-Lead ECG

ECG results are presented in a shift table (normal, abnormal not clinically significant, abnormal, clinically significant) to show the greatest change from baseline. All ECG results are presented in patient listings.

### Other Safety Variables

29T2A11 safety data is provided in listings.

ECOG PS and change from Baseline in ECOG PS are summarized at each scheduled visit that it is collected. Change from Baseline in ECOG PS is summarized as a continuous variable and as a categorical variable. A decrease of ≥1 point from Baseline is categorized as an “improvement” from Baseline. An increase of ≥1 point from Baseline is categorized as a “deterioration” from Baseline. Improvement, deterioration, and unchanged ECOG PS from Baseline is summarized as a categorical variable by treatment at each post-enrollment time point that ECOG PS is evaluated.

### Pharmacodynamic Analyses

Biomarkers are summarized for each time point, for change from Baseline and % change from Baseline. Correlation between pharmacodynamic markers and SQZ-PBMC-HPV is explored with descriptive and graphical methods.

Descriptive statistics (mean, standard deviation, median, minimum, maximum, and geometric mean) for each marker is reported. Graphs of individual values over time according to dose group is presented.

### Example 2. Analysis of Cohorts 1-3

The Cell Squeeze® technology has demonstrated robust abilities to deliver antigens directly to the cytosol, thereby circumventing the cross-presentation process most vaccines rely on and enabling efficient MHC-I presentation and antigen-specific CD8 T cell activation. Preclinical data has demonstrated superior CD8 T cell activation and anti-tumor effects in vitro and in vivo.

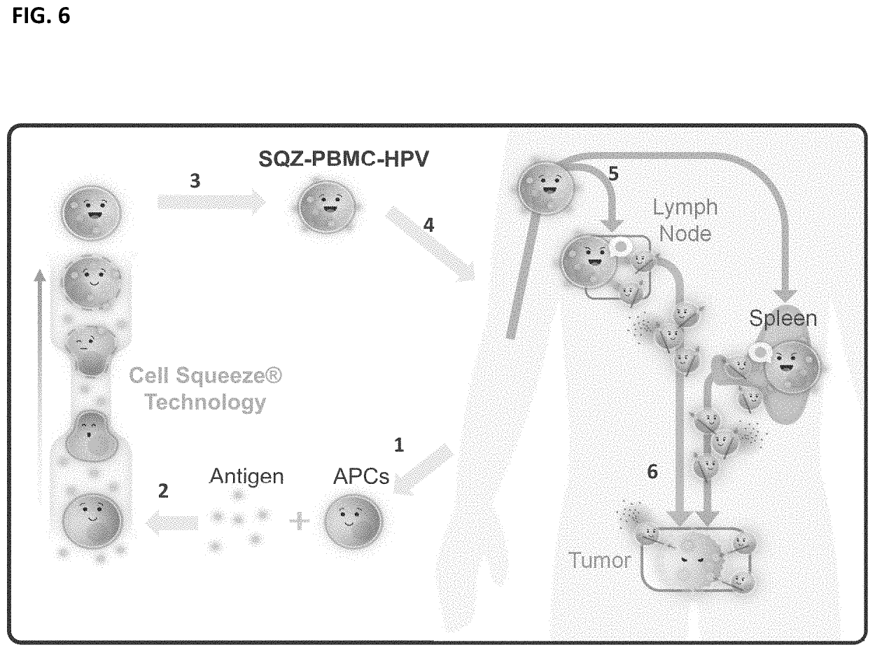

FIG. 6 illustrates the anticipated mechanism of SQZ-PBMC-HPV-101 investigational product generated by Cell Squeeze® technology, and that SQZ-PBMC-HPV Vaccines Directly Stimulate CD8 T cell response:
1. Peripheral blood mononuclear cells (PBMCs) are derived from the patient by leukapheresis and mixed with HPV16 E6 and E7 synthetic long peptides (SLPs)
2. The Cell Squeeze® technology uses rapid mechanical deformation of the PBMCs to disrupt their membrane temporarily and deliver the E6 and E7 antigen cargo directly to their cytoplasm
3. The resulting antigen presenting cells (APCs) are matured with CpG7909
4. SQZ-PBMC-HPV is not genetically modified by this process and is cryopreserved for storage and shipping to the patient
5. Preclinical data has demonstrated that murine SQZ™ APCs home to lymphoid organs to drive antigen-specific CD8 T cell activation
6. In preclinical models, the activated T cells home to the tumor site, induce tumor cell death, and form memory that provides long-term protection. SQZ-PBMC-HPV has demonstrated dramatic improvement in vaccine potency when benchmarked against other techniques in preclinical models.

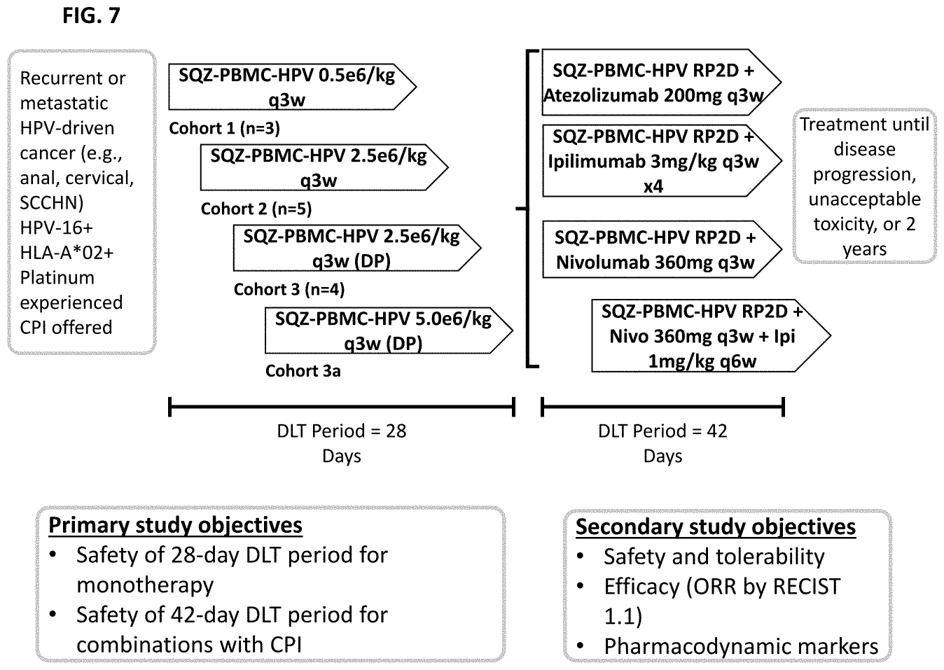

As shown in FIG. 7, the primary study objectives of the SQZ-PBMC-HPV-101 clinical investigation include Safety of 28-day DLT period for monotherapy and Safety of 42-day DLT period for combinations with checkpoint inhibitors (CPI). The secondary study objectives include safety and tolerability; efficacy (such as ORR by RECIST 1.1), and pharmacodynamics markers.

### Methods

SQZ-PBMC-HPV-101 has enrolled patients with incurable HPV16+ cancers progressing after unlimited prior lines of therapy (FIG. 7).

Eligible patients must have ECOG 0-1, adequate organ function and a lesion that could be biopsied with acceptable clinical risk.

Patients underwent a single leukapheresis at the study site and SQZ-PBMC-HPV batches were manufactured at Lonza, Portsmouth, N.H. SQZ-PBMC-HPV were not genetically modified. Vein to vein time took about 1 week.

Batch characterization was performed prior to batch release and comprised cell viability and induction of IFN-γ secretion.

Out-patient SQZ-PBMC-HPV was given IV q 3 weeks without a prior conditioning regimen; in each cohort the first and second patients were observed for 23 hours.

Double antigen priming (DP) was introduced with Cohort 3 and occurred on Cycle 1 Days 1 and 2.

DLT period was 28 days for monotherapy and 42 days for the combination phase.

Monotherapy dose escalation was done following the 3+3 rule. In all cohorts, a maximum of 12 patients will be enrolled.

Tumor biopsies were collected at baseline and on C2D8, processed into FFPE blocks, sectioned and IHC stained on an automated immunohistochemistry stainer using qualified mono-, dual- and tri-plex assays.

Treatment duration for each patient was determined by their assigned dose and the number of vials in their manufacturing batch.

Responses were assessed via RECIST 1.1 and iRECIST.

### Demographics of Patients, Disease Characteristics, Treatment Emergent Adverse Events (TEAEs)

Table 5 shows the demographics and disease characteristics of enrolled patients.

TABLE 5
Demographics and Disease Characteristics

| | Cohort | Cohort | Cohort | |
|---|---|---|---|---|
| | 1 | 2 | 3 | Total |
| | (n = 3) | (n = 5) | (n = 4) | (N = 12) |
| Age, years | 65.0 | 65.0 | 49.0 | 62.5 |
| Median (Min, Max) | (60, 68) | (54, 68) | (47, 66) | (47, 68) |
| Sex, n (%) | 3 | 3 (60.0) | 3 (75.0) | 9 (75.0) |
| Female | (100.0) | | | |
| Race, n (%) | 3 | 5 | 3 (75.0) | 11 (91.7) |
| Caucasian | (100.0) | (100.0) | | |
| Baseline ECOG score, n (%) | 2 | 3 (60.0) | 4 | 9 (75.0) |
| 1 | (66.7) | | (100.0) | |
| Baseline RMH score, n (%) | 0 | 3 (60.0) | 4 | 7 (58.3) |
| High (≥2) | | | (100.0) | |
| Time since diagnosis, | 46.06 | 15.80 | 52.78 | 39.51 |
| months | (43.0, | (12.0, | (21.2, | (12.0, |
| Median (Min, Max) | 51.5) | 75.2) | 80.3) | 80.3) |
| Site of primary tumor, n (%) | | | | |
| Anus | 2 (66.7) | 3 (60.0) | 2 (50.0) | 7 (58.3) |
| Cervix | 1 (33.3) | 0 | 1 (25.0) | 2 (16.7) |
| Head & Neck | 0 | 2 (40.0) | 1 (25.0) | 3 (25.0) |
| Site of Metastases, n (%) | 3 | 5 | 4 | 12 |
| | (100.0) | (100.0) | (100.0) | (100.0) |
| Liver Mets | 1 (33.3) | 3 (60.0) | 3 (75.0) | 7 (58.3) |
| Lung Mets | 1 (33.3) | 3 (60.0) | 1 (25.0) | 5 (41.6) |
| Other sites* | 2 (66.7) | 2 (40.0) | 2 (50.0) | 6 (50.0) |
| Number of Prior Lines, n | 4 (2, 5) | 3 (1, 7) | 3.5 | 4.0 (1, 7) |
| Median (Min, Max) | | | (3, 4) | |
| Prior Systemic Therapy, n | 3 | 5 | 4 | 12 |
| (%) | (100.0) | (100.0) | (100.0) | (100.0) |
| Chemotherapy | 3 | 4 (80.0) | 4 | 11 (91.7) |
| | (100.0) | | (100.0) | |
| Checkpoint Inhibitor | 3/3 | 3/4 | 3/4 | 9 (75.0) |
| Refractory to ICI (PD as | 2 (66.7) | 1 (20.0) | 2 (50.0) | 5 (41.6) |
| BOR) | | | | |
| Other | | | | |

Table 6 shows the disposition of enrolled patients.

TABLE 6
Patient Disposition

| | Cohort 1 | Cohort 2 | Cohort 3 | Total |
|---|---|---|---|---|
| | (n = 3) | (n = 5) | (n = 4) | (N = 12) |
| Dosed | 3 (100.0) | 5 (100.0) | 4 (100.0) | 12 (100.0) |
| No Longer on Treatment | 3 (100.0) | 5 (100.0) | 4 (100.0) | 12 (100.0) |
| Number of doses | 3-10 | 2-4 | 3-4 | 2-10 |
| received* (range) | | | | |
| Discontinued Treatment | 1 (33.3) | 4 (80.0) | 4 (100.0) | 9 (75.0) |
| Completed Treatment\hat{ } | 2 (66.7) | 1 (20.0) | 0 | 3 (25.0) |
| Progressive Disease | 1 (33.3) | 3 (60.0) | 3 (75.0) | 7 (58.3) |
| Death | 0 | 0 | 1 (25.0) | 1 (8.3) |
| Withdrawal of Consent | 0 | 1 (20.0) | 0 | 1 (8.3) |
| from Treatment Only | | | | |
| Currently in Follow-up | 2 (66.7) | 1 (20.0) | 2 (50.0) | 5 (41.7) |
| Discontinued Study | 1 (33.3) | 4 (80.0) | 2 (50.0) | 7 (58.3) |
| Death | 1 (33.3) | 3 (60.0) | 2 (50.0) | 6 (50.0) |
| Other¹ | 0 | 1 (20.0) | 0 | 1 (8.3) |
| All Deaths | 1 (33.3) | 4 (80.0) | 2 (50.0) | 7 (58.3) |
| Deaths within 30 Days | 0 | 0 | 2 (50.0) | 2 (16.7) |
| of Last Dose | | | | |

*Up to protocol v1.2 there was a limit of 3 doses. After v2.0 was approved patients could receive as many doses as available.
\hat{ }Treatment was limited to 3 administrations prior to protocol version 2.0.
¹Patient discharged to hospice; later died.

Table 7 shows the summary for causality TEAEs reported in 2 or more patients.

TABLE 7
All Causality TEAEs Reported in 2 or More Patients (any Grade)

| | Cohort 1 | Cohort 2 | Cohort 3 | Total |
|---|---|---|---|---|
| Preferred Term, n (%) | (n = 3) | (n = 5) | (n = 4) | (N = 12) |
| Dyspnea | 0 | 2 (40.0) | 2 (50.0) | 4 (33.3) |
| Dehydration | 1 (33.3) | 1 (20.0) | 1 (25.0) | 3 (25.0) |
| Diarrhea | 0 | 2 (40.0) | 1 (25.0) | 3 (25.0) |
| Dizziness | 1 (33.3) | 1 (20.0) | 1 (25.0) | 3 (25.0) |
| Hypotension | 2 (66.7) | 1 (20.0) | 0 | 3 (25.0) |
| Urinary Tract Infection | 0 | 2 (40.0) | 1 (25.0) | 3 (25.0) |
| Abdominal Distension | 1 (33.3) | 0 | 1 (25.0) | 2 (16.7) |
| Anemia | 0 | 1 (20.0) | 1 (25.0) | 2 (16.7) |
| Anxiety | 1 (33.3) | 1 (20.0) | 0 | 2 (16.7) |
| Depression | 1 (33.3) | 0 | 1 (25.0) | 2 (16.7) |
| Fatigue | 0 | 2 (40.0) | 0 | 2 (16.7) |
| Flushing | 1 (33.3) | 0 | 1 (25.0) | 2 (16.7) |
| Hypomagnesemia | 1 (33.0) | 0 | 1 (25.0) | 2 (16.7) |
| Hyponatremia | 0 | 0 | 2 (50.0) | 2 (16.7) |
| Muscular weakness | 1 (33.0) | 0 | 1 (25.0) | 2 (16.7) |
| Non-cardiac chest pain | 0 | 0 | 2 (50.0) | 2 (16.7) |
| Edema peripheral | 0 | 0 | 2 (50.0) | 2 (16.7) |
| Pleural effusion | 0 | 0 | 2 (50.0) | 2 (16.7) |
| Procedural pain | 0 | 0 | 2 (50.0) | 2 (16.7) |
| Urinary retention | 1 (33.0) | 0 | 1 (25.0) | 2 (16.7) |
| Weight decreased | 0 | 1 (20.0) | 1 (25.0) | 2 (16.7) |

### Manufacturability of SQZ-PBMC-HPV

The manufacturing process was <24 hrs per patient and allowed for multiple doses produced from one run. Dramatic improvement in manufacturing and release testing supports a vein-to-vein time of about a 1 week.

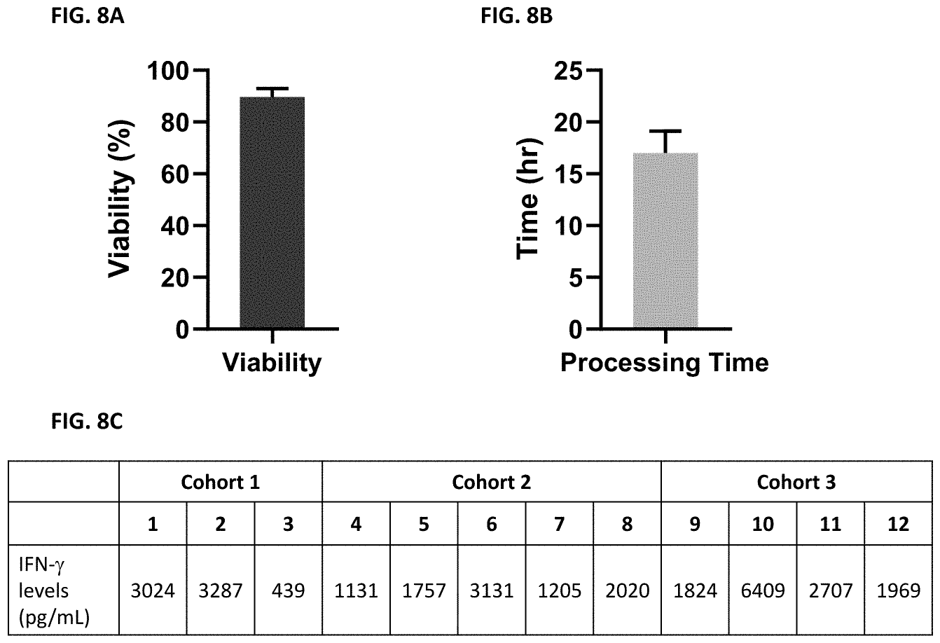

As shown in FIGS. 8A and B, the manufacturing of SQZ-PBMC-HPV showed an average viability of about 90%, and the average of end-to-end process time was less than 24 hours. As shown in FIG. 8C, SQZ-PBMC-HPV generated from all patient batches presented the HPV antigen, as illustrated by the ability to induce T cell activity, assayed in responsive T-cell IFN-γ secretion.

### Treatment Outcome

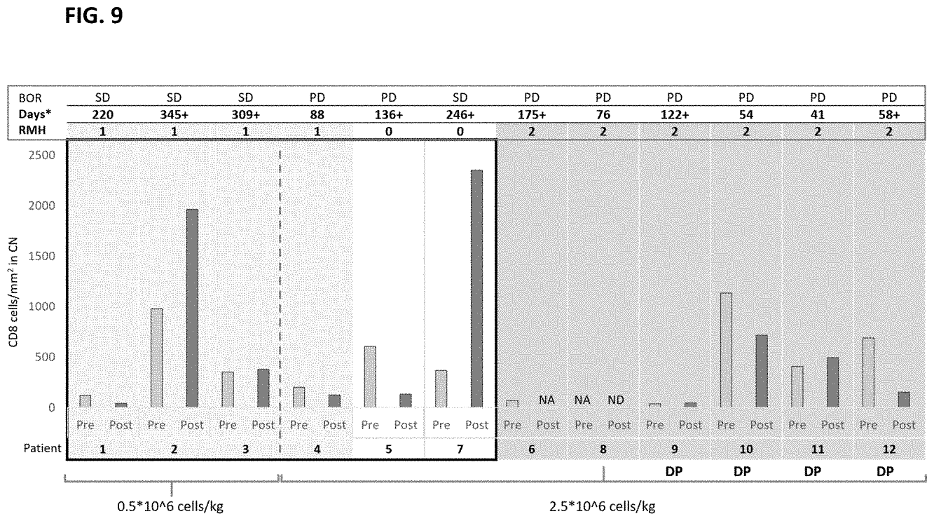

The treatment outcome is summarized in FIG. 9, showing the summary of best overall response (BOR), Survival on study (Days) and Royal Marsden Hospital (RMH)1 Score across all cohorts (shading based on score). As shown in FIG. 9, All Cohort 3 patients had a RMH score of 2 and no significant increase in CD8 tumor infiltrating lymphocytes.

### Patient Case Study

Patients with different tumor burdens were selected for further case studies.

### Case Study Patient 2

A 65-year-old woman with cervix carcinoma was enrolled 3.5 years after diagnosis following treatment with (1) cisplatin/paclitaxel/bevacizumab [BOR=CR] and (2) Pembrolizumab [BOR=PD] in Cohort 1 (0.5e6/kg q3w).

Low tumor burden, ECOG of 0 and Royal Marsden Hospital (RMH) 1 Score of 1 at baseline; on study for 10+ months.

Tumor burden 1 target lesion (TL) (15 mm SOD) and 3 NTL (2 lymph nodes, lung). Best overall response by RECIST 1.1: SD.

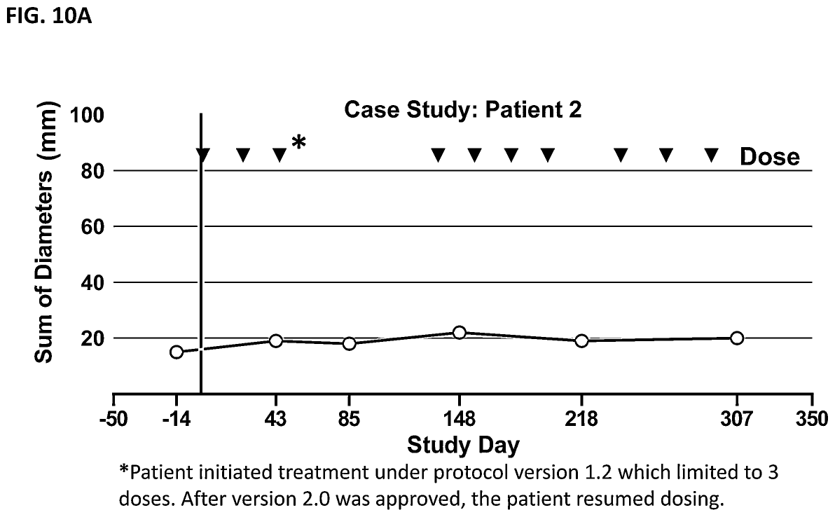

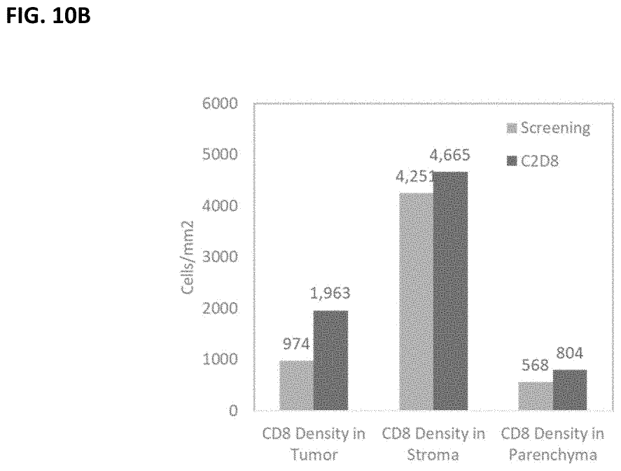

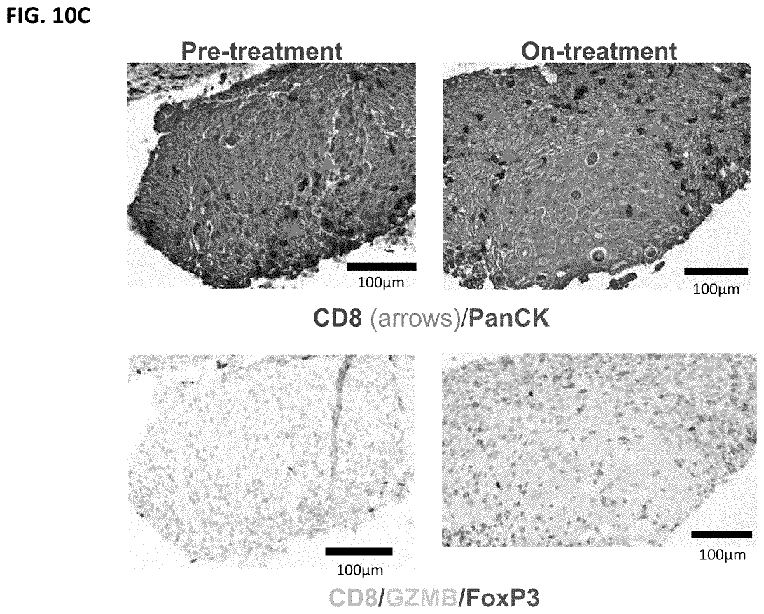

FIG. 10 shows a reduced tumor growth kinetic (FIG. 10A), whereas results from IHC image analyses show a 2-fold increase in CD8 TIL in the central tumor (FIG. 10B, C2D8 compared to screening) and examples of IHC images further demonstrate the increase in CD8 TIL (FIG. 10C, arrowheads).

### Case Study Patient 7

A 67-year-old male with head and neck cancer was enrolled 1 year after diagnosis following treatment with carboplatin/5FU/pembrolizumab (BOR=PR) in Cohort 2 (2.5e6/kg q3w).

High tumor burden, ECOG of 1 and Royal Marsden Hospital (RMH) 1 Score of 0 at baseline; on study for 3 months.

Tumor burden 2 TLs: 69 mm SOD; 1 NTL (lymph node). Best overall response by RECIST 1.1: SD.

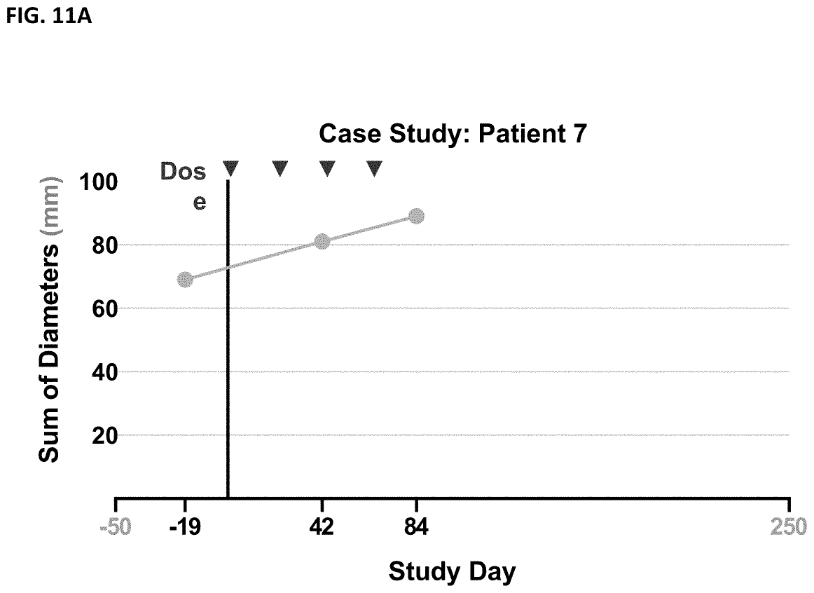

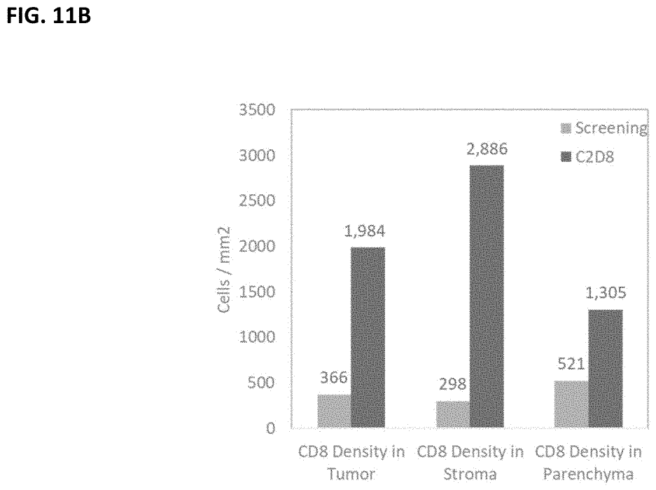

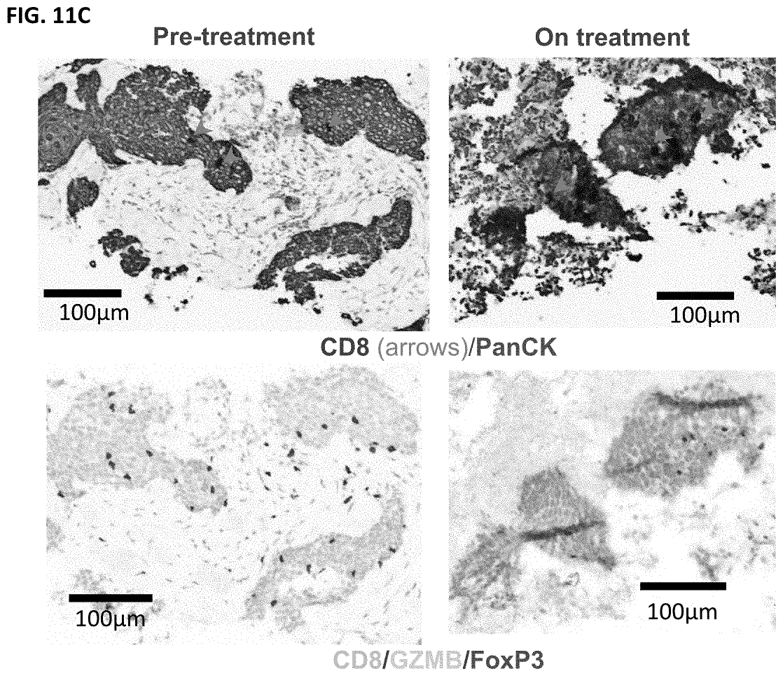

FIG. 11 shows a reduced tumor growth kinetic (FIG. 11A), whereas results from IHC image analyses show a 6-fold increase in CD8 TIL in the central tumor on treatment (FIG. 11B, C2D8 compared to screening) and examples of IHC images demonstrating the significant increase in CD8 TIL (FIG. 11C, arrowheads).

### CONCLUSIONS

Safety and tolerability
SQZ-PBMC-HPV was safe and well-tolerated at all dose levels with patients receiving 2 to 10 doses.
No DLTs or G>2 treatment-related SAEs observed.

Manufacturability
All batches were produced under cGMP consistent with specifications, yielded multiple cryopreserved doses in <24 hrs, and allowed a vein-to-vein time of about 1 week.
The product characterization assay based on T cell activity (IFN-γ secretion) confirmed antigen presentation in all patient batches independent of medical history or RMH Phase 1 score of individual patients.

Biomarker
Increased immune activity were observed in patients, such as patient 2 and 7 in case studies. Six out of 9 patients in the high-dose cohorts had RMH Phase 1 score of 2 at baseline—reflecting advanced disease and immuno-compromised patients.
Based on the 2 case studies, less advanced patients with lower tumor burden, such as patient 2, might have a higher likelihood of clinical benefit.

Future Developments: The initiation of the safety combination phase with immune checkpoint inhibitors is expected once assessment of the current dose level is complete.

### Example 3. Biomarker Analysis of Tumors

Tumor samples from patients enrolled in the clinical study described in Example 2 were evaluated for expression of biomarkers. Tumor biopsies were collected at baseline and at cycle 2, day 8 (C2D8), processed into FFPE blocks, sectioned and IHC stained on an automated immunohistochemistry stainer using qualified mono-, dual- and tri-plex assays.

Cohorts described in this Example are shown in Table 8.

TABLE 8
Cohorts for biomarker study

| Cohort | Treatment | Boost |
|---|---|---|
| Cohort 1 | 0.5 × 10⁶ cells/kg | Single Prime (SP) |
| Cohort 2 | 2.5 × 10⁶ cells/kg | Single Prime (SP) |
| Cohort 3 | 2.5 × 10⁶ cells/kg | Double Prime (DP) |
| Cohort 3a | 5.0 × 10⁶ cells/kg | Double Prime (DP) |

### Results

### Adverse Events

SQZ-PBMC-HPV is considered safe and well-tolerated. Adverse events at the time of the Biomarker Studies is presented in Table 9.

TABLE 9

| | Cohort 1 | Cohort 2 | Cohort 3 | Cohort 3a | Total |
|---|---|---|---|---|---|
| | (n = 3) | (n = 5) | (n = 4) | (n = 6) | (n = 16) |
| Related AEs* | 3 (100%) | 4 (80%) | 2 (50%) | 5 (83%) | 14 (78%) |
| Related Grade | 0 | 1 (20%) | 0 | 0 | 1 (6%) |
| 3 + AEs | | | | | |
| Related Serious | 1 (33%) | 0 | 0 | 0 | 1 (6%) |
| AEs | | | | | |
| AEs of Special | 1 | 0 | 1 | 0 | 2 (11%) |
| Interest | | | | | |
| Dose-limiting | 0 | 0 | 0 | 0 | 0 |
| toxicity | | | | | |
| Related AEs | 0 | 0 | 0 | 0 | 0 |
| leading to dc | | | | | |
| Fatal Related | 0 | 0 | 0 | 0 | 0 |
| AEs | | | | | |

*Common related AEs (>1 patient): fatigue, hypotension, infusion related reaction, nausea, pruritus. M = millions, SP = single-prime, DP = double-prime, AE = adverse events

No subject met pre-specified DLT criteria

Three AESIs reported in 2 patients:

Grade 2 Cytokine Release Syndrome (CRS) and Grade 1 and 2 Immune-Related Reaction

No related Grade≥3 SAEs reported:

Grade 2 (related)—CRS (1 pt)

Related AE Grade 3+ in 1 patient (anemia)

One patient had one dose delayed due to nasal congestion

No DLTs, no related AEs leading to discontinuation and no fatal related AEs observed.

### Biomarker Studies

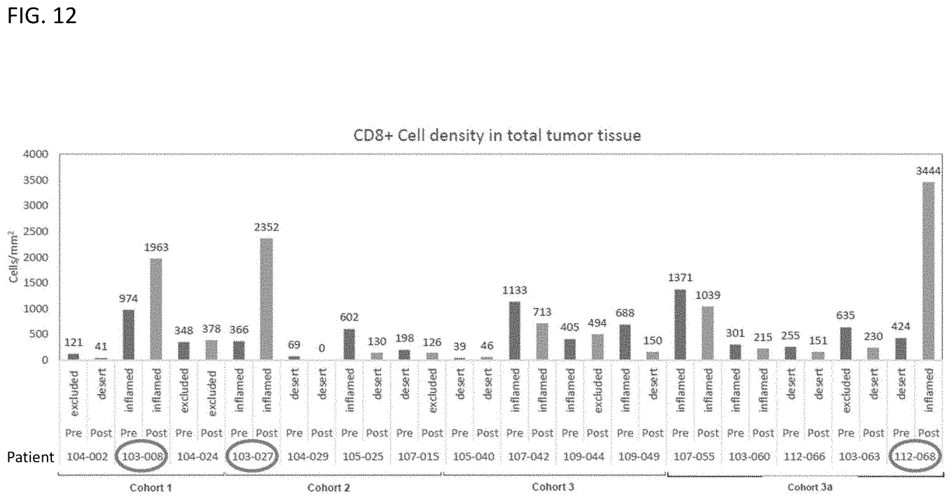

The CD8+ cell density in total tumor tissue is shown in FIG. 12. Tumors were also assessed as desert (lack of immune cells), excluded (immune cells on periphery only), or inflamed.

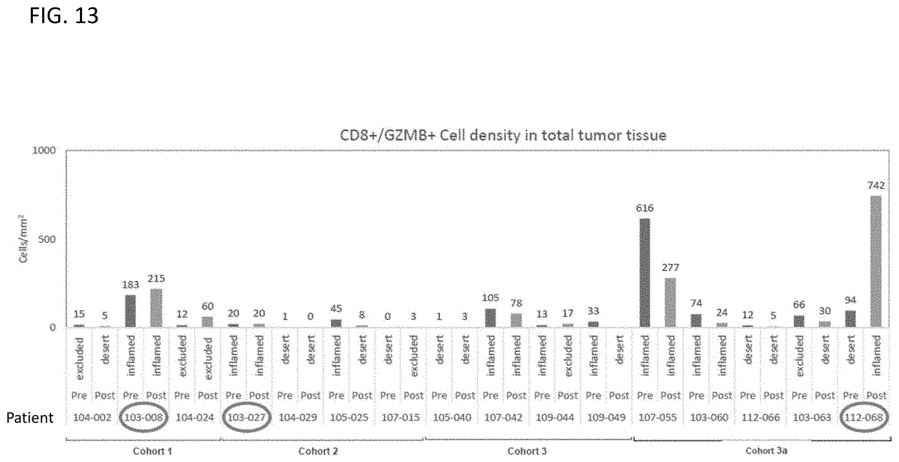

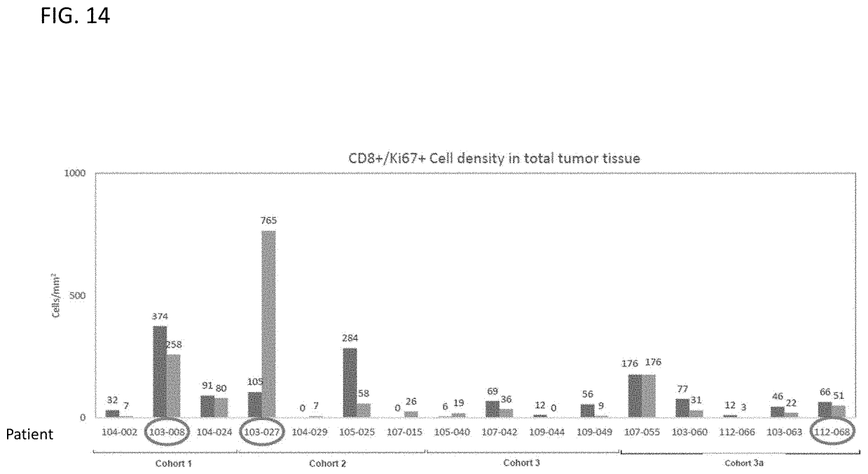

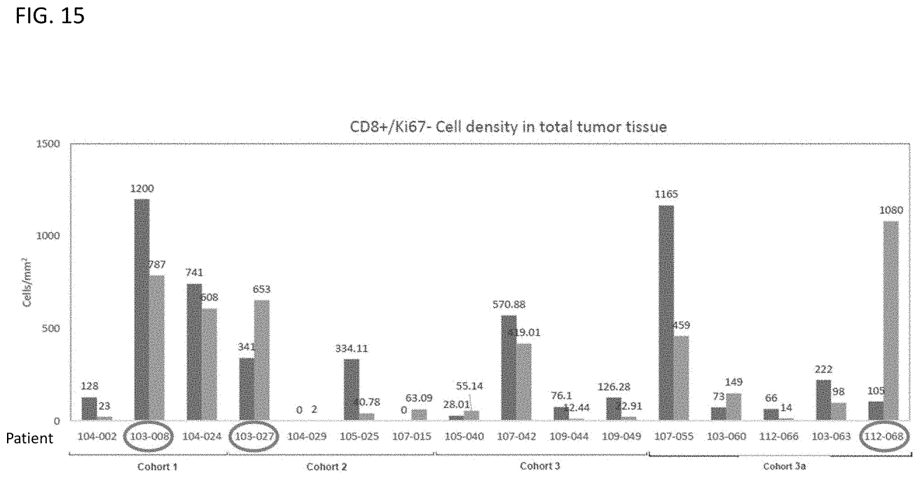

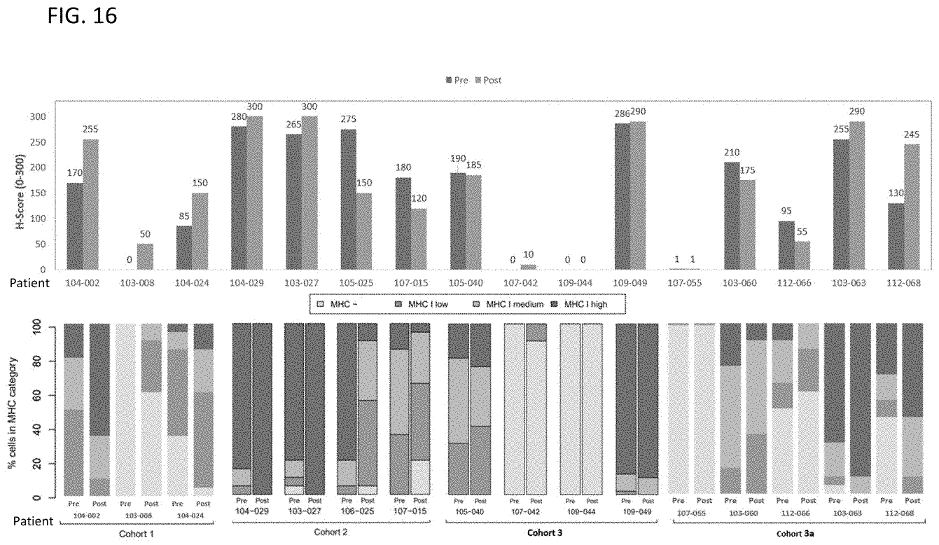

The density of CD8+ cell density among granzyme B (GZMB+) cells is shown in FIG. 13. The density of CD8+/Ki67+ cells is shown in FIG. 14 and the density of CD8+/Ki67− cells is shown in FIG. 15. MHC-1 expression is shown in FIG. 16 based on H-Score. The % cells in the following MHC categories, MHC-, MHC-1 low, MHC-1 medium, and MHC-1 high is also shown. The scoring includes the intensity of the staining and the proportion of the cells staining positively at each intensity. The staining signal is divided into 4 different intensity categories: 0: no staining; 1+: weak staining (visible at high power magnification); 2+: intermediate (or moderate) staining (visible at low power magnification); 3+: strong staining (striking even at low power magnification)).

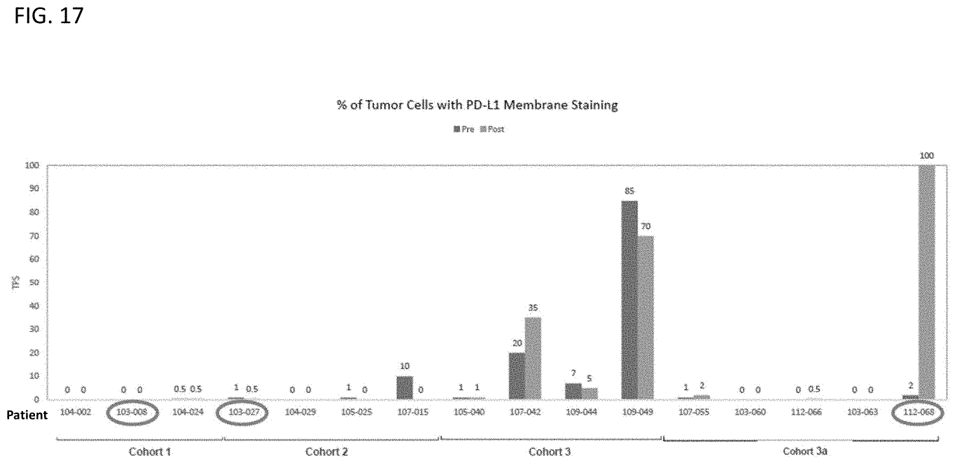

The percentage of tumor cells expressing PD-L1 is shown in FIG. 17.

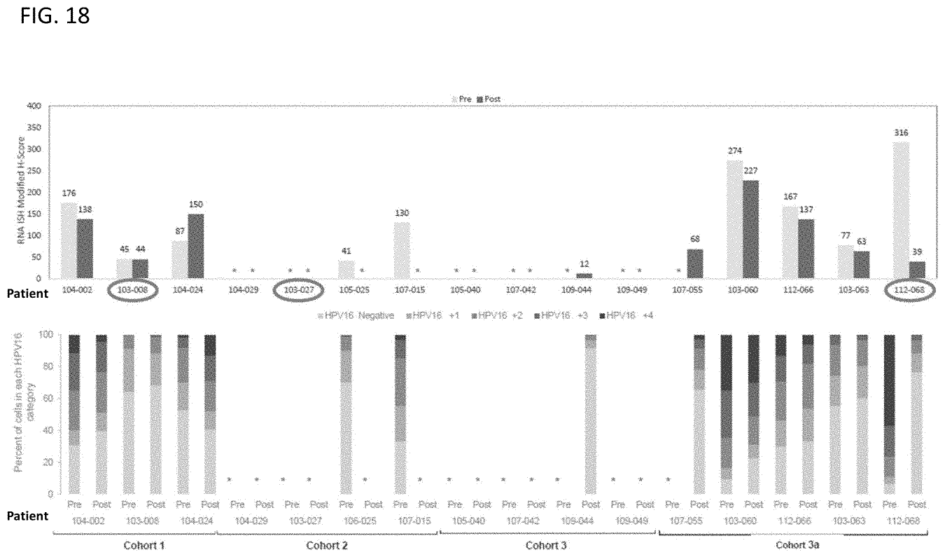

The expression of HPV E6 is shown in FIG. 18 based on H-Score. The % cells in the following HPV categories, HPV-, HPV16+1, HPV16+2 HPV16+3, and HPV16+4 is also shown. Cells with between 1-3 probe spots=+1, 4-9 spots=+2, 10-13 spots=+3, and >14 spots=+4.

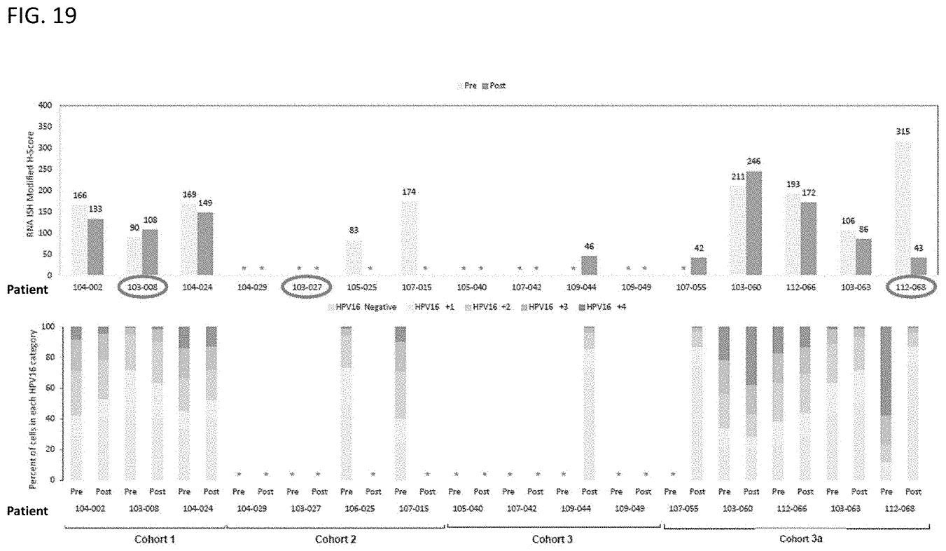

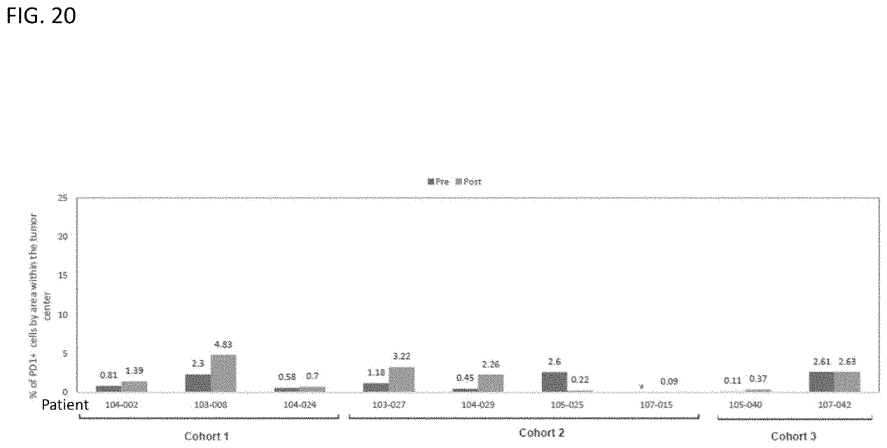

The expression of HPV E7 is shown in FIG. 19 based on H-Score. The % cells in the following HPV categories, HPV-, HPV16+1, HPV16+2 HPV16+3, and HPV16+4 is also shown. Cells with between 1-3 probe spots=+1, 4-9 spots=+2, 10-13 spots=+3, and >14 spots=+4. Biopsies for patients in cohorts 1-3 were evaluated for expression of PD1 (FIG. 20).

### Summary of Selected Patients

### 112-068 (Cohort 3a)

52-year-old man with squamous cell carcinoma of the oropharynx enrolled in the highest dose cohort (5.0M cells/kg DP). Initial diagnosis 3.7 years ago, following 6 prior lines of systemic therapy cisplatin, carbo/5FU/pembro (BOR=PD), docetaxel, cetuximab, anti-TGFβ/pembro (BOR=PD), cetuximab/paclitaxel. Large primary lesion with significant symptoms burden. Received all 7 doses of SQZ-PBMC-HPV, with few low grade related AEs (G1 flushing, G1 fatigue). Day-28 on treatment biopsy demonstrated a 8 fold increase in CD8 infiltrating the tumor, with reflex increase in PD-L1 expression. Radiographic response, including confirmed CR on target lesion (mediastinal lymph node (RECIST 1.1) with a new dermal lesion at the last tumor assessment. Symptomatic improvement (dysphagia) and macroscopic improvement of the lesion on physical examination.

Tumor went from desert (no immune cells) to inflamed. Patient showed the highest CD8 infiltration (and effector molecule GZMB expression on CD8+ cells). There was no change in proliferating (Ki67+) CD8+ cell. An increase in MHC-I and PD-L1 was observed. Patient showed the highest E6 and E7 expression in tumor at baseline and greatest post treatment change.

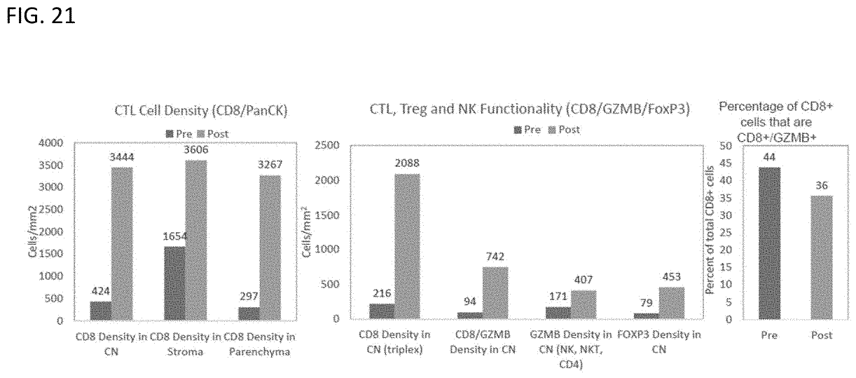

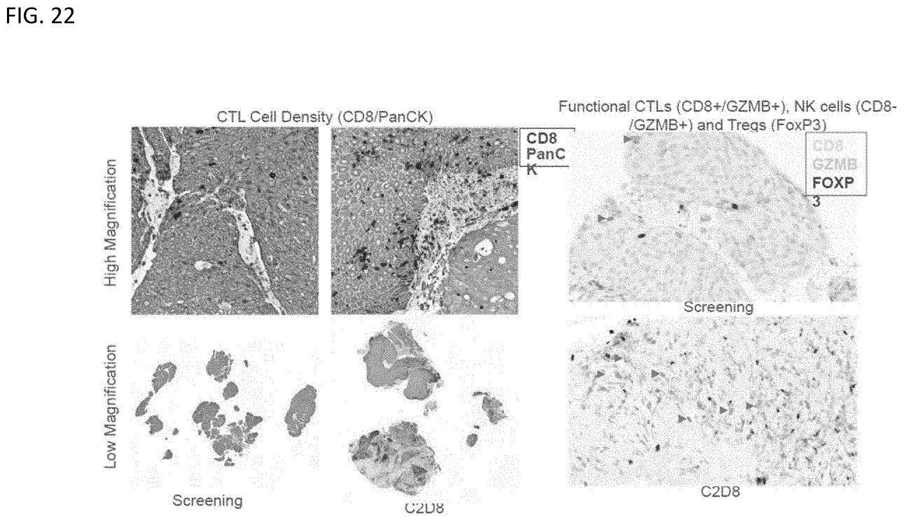

A more detailed analysis of the immune phenotype of the central tumor of this patient is shown in FIG. 21. Right panel shows CD8+ cell density in the stroma and parenchyma of the tumor. The middle panel shows densities of CTL, Treg and NK functionality of immune cells in the tumor based on expression of CD8, GZMB and FoxP3 in a triplex assay. The right panel shows the percentage of cells that are CD8+ and GZMB+. These results are further demonstrated by immunohistochemistry (FIG. 22).

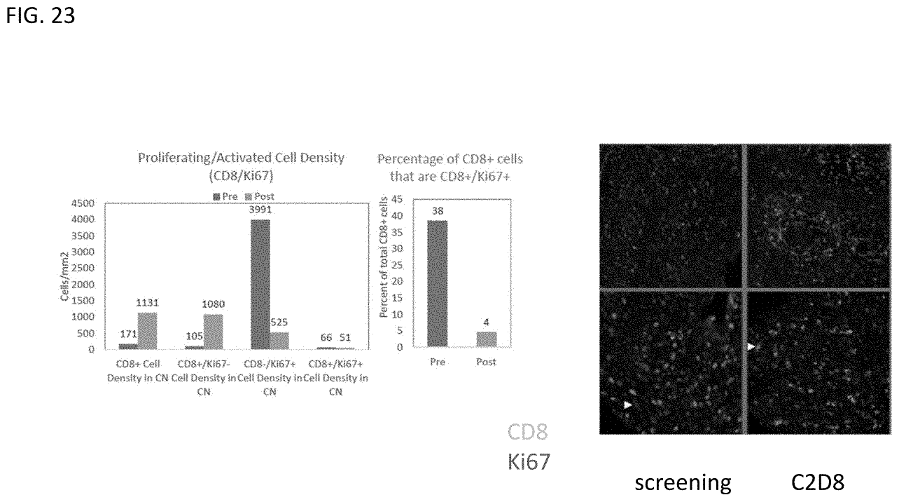

A more detailed analysis of the density of proliferating/activated CD8+ cells is shown in FIG. 23.

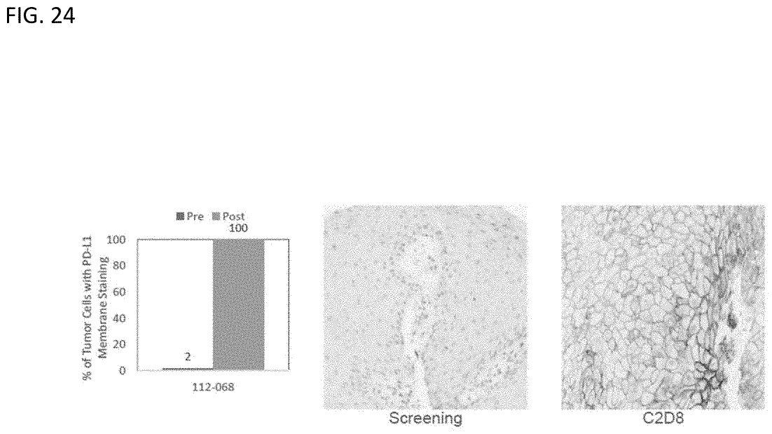

As shown in FIG. 24, an increase in expression of PD-L1 was observed.

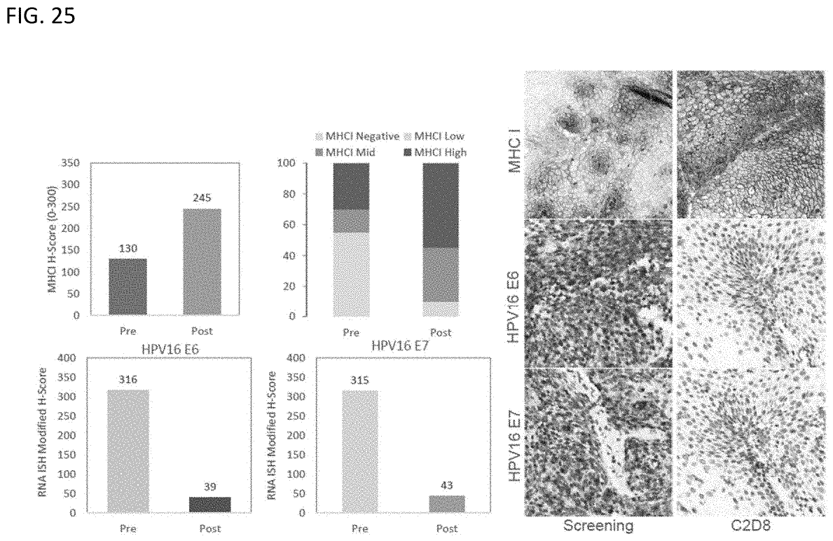

As shown in FIG. 25, the number of MHC-1 cells in the tumor increased by C2D8 including an increase in the proportion of MHC-1 high cells (left panel, top). In contrast, the number of cells expressing HPV16 E6 and HPV16 E7 decreased by C2D8 (left panel, bottom). These results are also shown by immunohistochemistry (right panel).

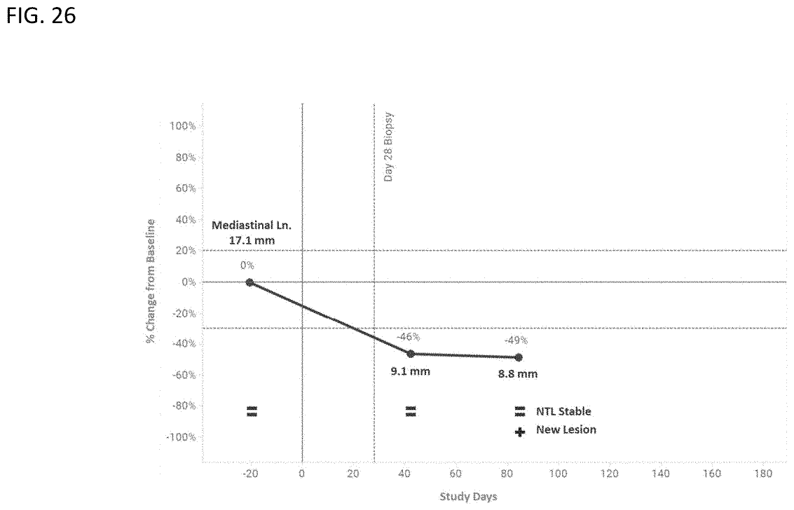

Tumor growth kinetics are shown in FIG. 26.

### 103-027 (Cohort 2)

The tumor of this patient was inflamed at the beginning of the study and inflammation had increased by C2D8. There was a significant increase in CD8 infiltrate (but no change in effector molecule GZMB expression on CD8+ cells). This patient had the highest proliferating CD8+ cells as measured by CD8+/Ki67+. There was no significant change in MHC-I and PD-L1 expression.

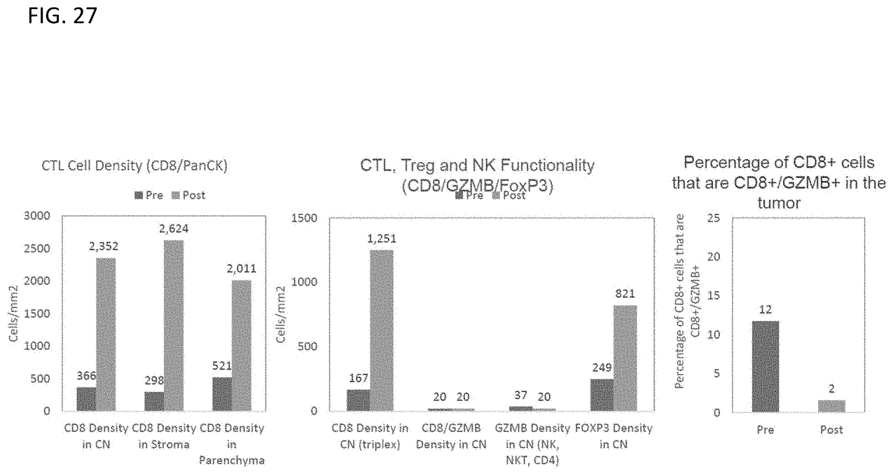

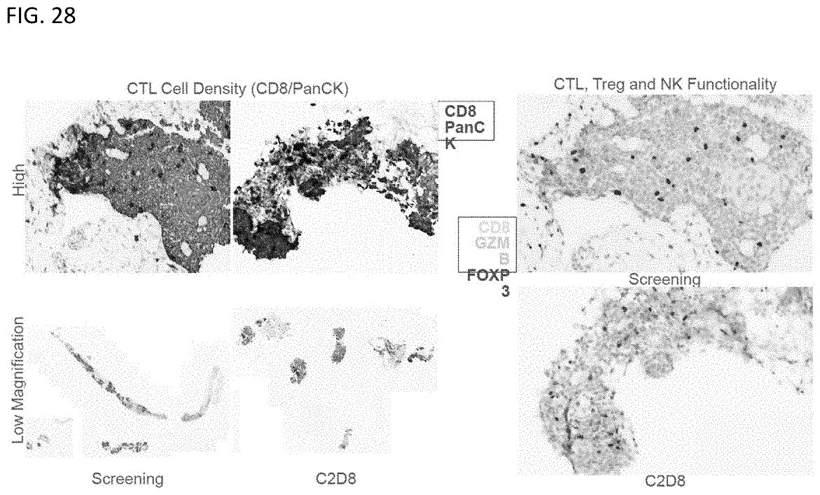

A more detailed analysis of the immune phenotype of this patient is shown in FIG. 27. Right panel shows CD8+ cell density in central tumor and in the stroma and parenchyma of the tumor. The middle panel shows densities of CTL, Treg and NK functionality of immune cells in the tumor based on expression of CD8, GZMB and FoxP3 in a triplex assay. The right panel shows the percentage of cells that are CD8+ and GZMB+. These results are further demonstrated by immunohistochemistry (FIG. 28).

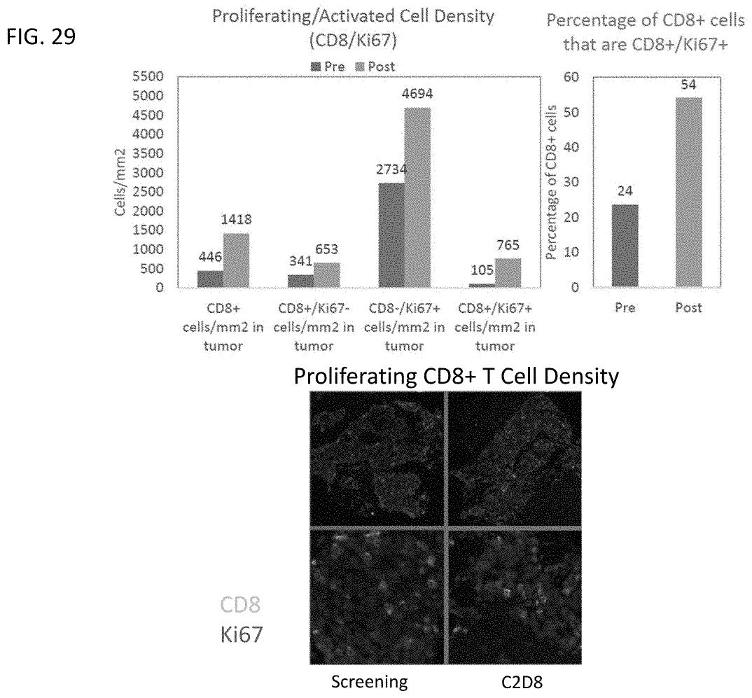

A more detailed analysis of the density of proliferating/activated CD8+ cells is shown in FIG. 29.

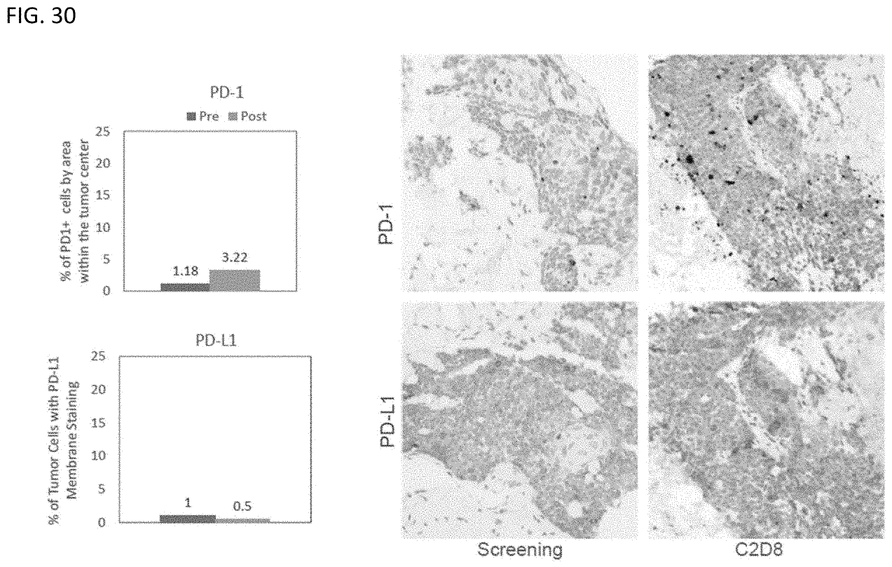

As shown in FIG. 30, expression of PD-1 and PD-L1 remained about the same.

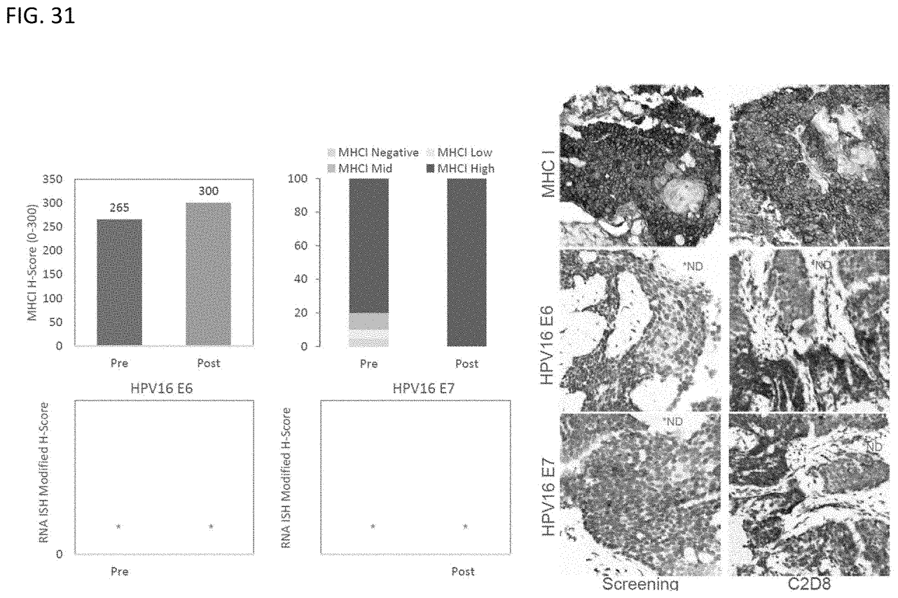

As shown in FIG. 31, the number of MHC-1 cells in the tumor remained about the same\ an increase in the proportion of MHC-1 high cells (left panel, top). In contrast, the number of cells expressing HPV16 E6 and HPV16 E7 decreased by C2D8 (left panel, bottom). These results are also shown by immunohistochemistry (right panel).

### 103-008 (Cohort 1)

The tumor of this patient was inflamed at the beginning of the study and inflammation had increased by C2D8. A trend towards increasing CD8 infiltration was observed but no change in effector molecule GZMB expression on CD8+ cells. No change in proliferating (Ki67+) CD8+ cell was observed. No change in PD-L1 was observed. There was no change in E6 and E7 expression in tumor at Baseline or post treatment.

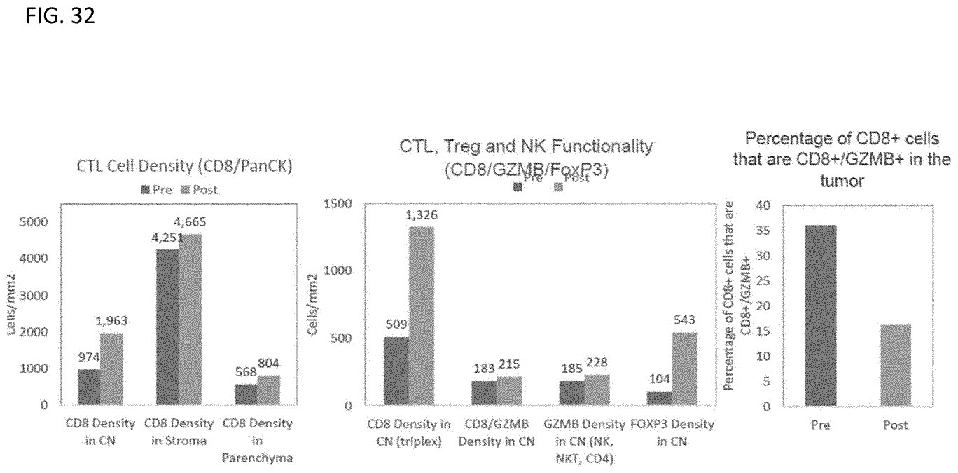

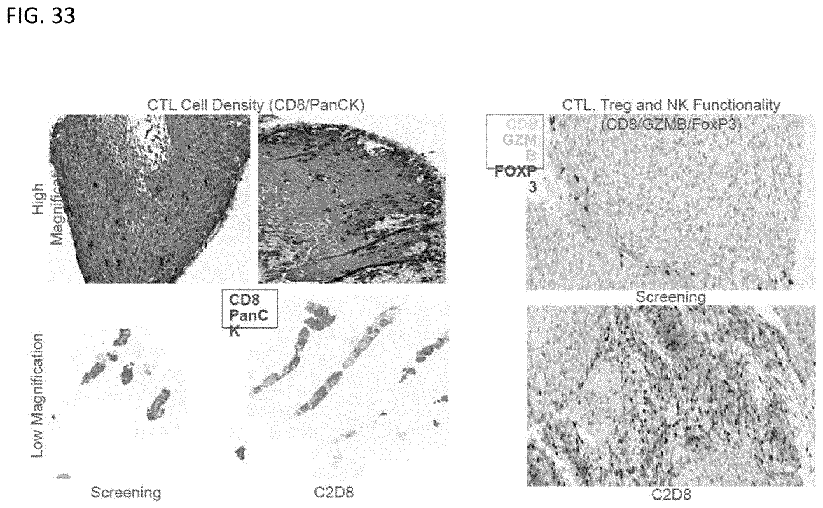

A more detailed analysis of the immune phenotype of this patient is shown in FIG. 32. Right panel shows CD8+ cell density in central tumor and in the stroma and parenchyma of the tumor. The middle panel shows densities of CTL, Treg and NK functionality of immune cells in the tumor based on expression of CD8, GZMB and FoxP3 FoxP3 in a triplex assay. The right panel shows the percentage of cells that are CD8+ and GZMB+. These results are further demonstrated by immunohistochemistry (FIG. 33).

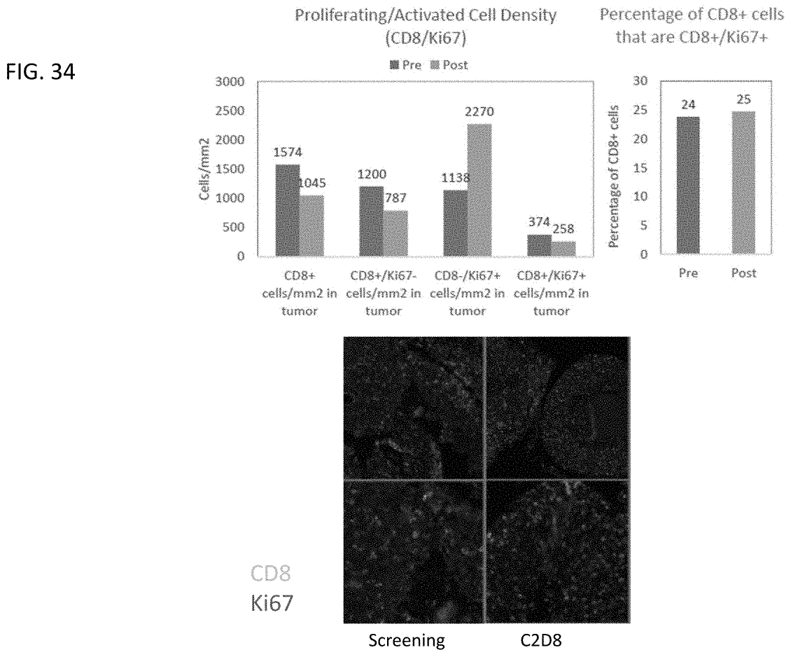

A more detailed analysis of the density of proliferating/activated CD8+ cells is shown in FIG. 34.

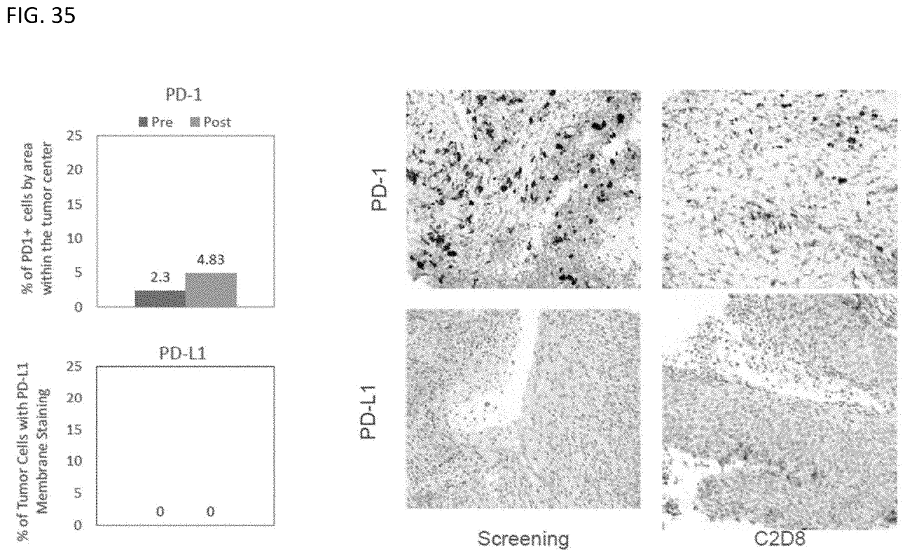

As shown in FIG. 35, expression of PD-1 remained about the same at C2D8. No PD-L1 was detected, either at initial screening or at C2D8.

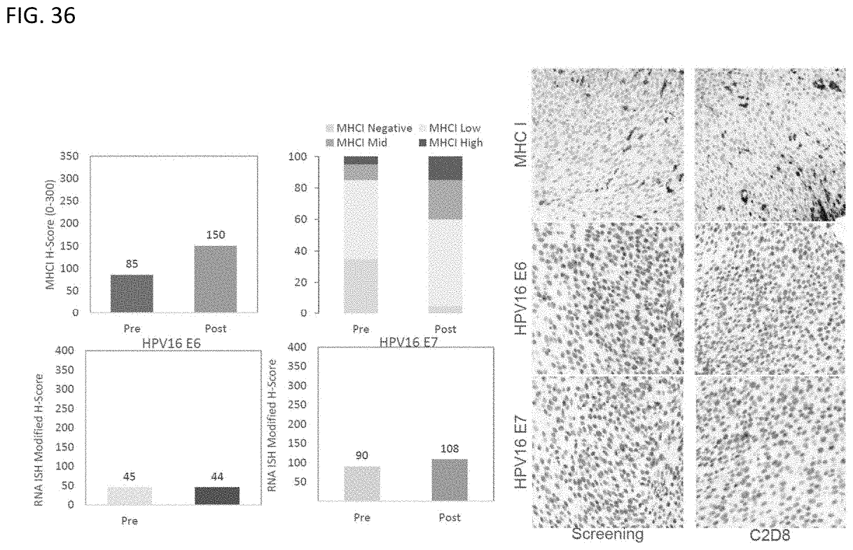

As shown in FIG. 36, the number of MHC-1 cells in the tumor remained about the same (left panel, top) as did the number of cells expressing HPV16 E6 and HPV16 E7 (left panel, bottom). These results are also shown by immunohistochemistry (right panel).

SEQUENCES.

| SEQ | | |
|---|---|---|
| ID | | |
| NO | Sequence | Description |
| 1 | TIHDIILECV | HPV16-E6(29-38), human |
| | | epitope |
| 2 | EVYDFAFRDL | HPV16-E6(48-57), murine |
| | | epitope |
| 3 | YMLDLQPETT | HPV16-E7(l1-20), human |
| | | epitope |
| 4 | RAHYNIVTF | HPV16-E7(49-57), murine |
| | | epitope |
| 5 | LPQLSTELQT | HPV16-E6(19-28) N-terminal |
| | | polypeptide, human |
| 6 | QLCTELQT | HPV16-E6(21-28) N-terminal |
| | | polypeptide, human |
| 7 | KQQLLRR | HPV16-E6(41-47) N-terminal |
| | | polypeptide, native murine |
| 8 | VYSKQQLLRR | HPV16-E6(38-47) N-terminal |
| | | polypeptide, classic murine |
| 9 | MHGDTPTLHE | HPV16-E7(1-10) N-terminal |
| | | polypeptide, human |
| 10 | GQAEPD | HPV16-E7(43-48) N-terminal |
| | | polypeptide, murine |
| 11 | YSKQQLLRREVYDFAF | HPV16-E6(39-54) C-terminal |
| | | polypeptide, human |
| 12 | YCKQQLL | HPV16-E6(39-45) C-terminal |
| | | polypeptide, human |
| 13 | CIVYRDGN | HPV16-E6(58-65) C-terminal |
| | | polypeptide, native murine |
| 14 | SIVYRDGNPYAVSDK | HPV16-E6(58-72) C-terminal |
| | | polypeptide, classic murine |
| 15 | DLYCYEQLNDSSEEE | HPV16-E7(21-35) C-terminal |
| | | polypeptide, human |
| 16 | CCKCDSTLRLCVQSTHVDIR | HPV16-E7(58-77 C-terminal |
| | | polypeptide, native murine |
| 17 | SSKSDSTLRLSVQSTHVDIR | HPV16-E7(58-77) C-terminal |
| | | polypeptide, classic murine |
| 18 | LPQLSTELQTTIHDIILECVYSKQ | HPV16-E6( 19-54) SLP, |
| | QLLRREVYDFAF | human |
| 19 | QLCTELQTTIHD11LECVYCKQQLL | HPV16-E6(21-45) SLP, |
| | | human |
| 20 | KQQLLRREVYDFAFRDLCIVYRDGN | HPV16-E6(41-65) SLP, native |
| | | murine |
| 21 | VYSKQQLLRREVYDFAFRDLSIVYR | HPV16-E6(38-72) SLP, classic |
| | DGNPYAVSDK | murine |
| 22 | MHGDTPTLHEYMLDLQPETTDLYCY | HPV16-E7(1-35) SLP, human |
| | EQLNDSSEEE | |
| 23 | QLCTELQTYMLDLQPETTYCKQQLL | HPV16-E7.6 SLP, human |
| 24 | GQAEPDRAHYNIVTFCCKCDSTLRL | HPV16-E7(43-77) SLP, native |
| | CVQSTHVDIR | murine |
| 25 | GQAEPDRAHYNIVTFSSKSDSTLRL | HPV16-E7(43-77) SLP, classic |
| | SVQSTHVDIR | murine |
| 26 | ggGGTCAACGTTGAgggggg | ODN 1585 (Class A, mouse- |
| | Bases shown in capital | specific) |
| | letters are phosphodiester, | |
| | and those in lower case are | |
| | phosphorothioate | |
| 27 | ggGGGACGA:TCGTCgggggg | ODN 2216 (Class A, human- |
| | Bases shown in capital | selective) |
| | letters are phosphodiester, | |
| | and those in lower case are | |
| | phosphorothioate | |
| 28 | gggGACGAC:GTCGTGgggggg | ODN 2336 (Class A, human |
| | Bases shown in capital | preferred) |
| | letters are phosphodiester, | |
| | and those in lower case are | |
| | phosphorothioate | |
| 29 | tccatgacgttcctgatgct | ODN 1668 (Class B, mouse |
| | Bases shown in capital | specific) |
| | letters are phosphodiester, | |
| | and those in lower case are | |
| | phosphorothioate | |
| 30 | tccatgacgttcctgacg | ODN 1826 (Class B, mouse |
| | tt | specific) |
| | Bases are phosphorothioate | |
| 31 | tcgtcgttttgtcgtttt | ODN 2006 (Class B, human |
| | gtcgtt | selective) |
| | Bases are phosphorothioate | |
| 32 | tcg tcg ttg tcg ttt | ODN 2007 (Class B, |
| | tgt cgt t | bovine/porcine) |
| | Bases are phosphorothioate | |
| 33 | tcg acg ttc gtc gtt | ODN BW006 (Class B, |
| | cgt cgt tc | human & mouse) |
| | Bases are phosphorothioate | |
| 34 | tcg cga cgt tcg ccc | ODN D-SL01 (Class B, |
| | gac gtt cgg ta | multispecies) |
| | Bases are phosphorothioate | |
| 35 | tcgtcgttttcggcgc:gcgccg | ODN 2395 (Class C, |
| | Bases are phosphorothioate | human/mouse) |
| 36 | tcgtcgtcgttc:gaacgacgttgat | ODN M362 (Class C, |
| | Bases are phosphorothioate | human/mouse) |
| 37 | tcg cga acg ttc gcc | ODN D-SL03 (Class C, |
| | gcg ttc gaa cgc gg | multispecies) |
| | Bases are phosphorothioate | |
| 38 | MHGDTPTLHEYMLDLQPETTDLYCYE | E7 |
| | QLNDSSEEE | |
| 39 | LYCYEQLNDSSEEEDEIDGPAGQAEP | E7 |
| | DRAHYNIVT | |
| 40 | GQAEPDRAHYNIVTFCCKCDSTLRLC | E7 |
| | VQSTHVDIR | |
| 41 | TLRLCVQSTHVDIRTLEDLLMGTLGI | E7 |
| | VCPICSQKP | |
| 42 | MHQKRTAMFQDPQERPRKLPQLCTEL | E6 |
| | QTTIHD | |
| 43 | LPQLCTELQTTIHDIILECVYCKQQL | E6 |
| | LRREVY | |
| 44 | KQQLLRREVYDFAFRDLCIVYRDGN | E6 |
| 45 | RDLCIVYRDGNPYAVCDKCLKFYSKI | E6 |
| 46 | DKCLKFYSKISEYRHYCYSLYGTTL | E6 |
| 47 | HYCYSLYGTTLEQQYNKPLCDLLIR | E6 |
| 48 | YGTTLEQQYNKPLCDLLIRCINCQKP | E6 |
| | LCPEEK | |
| 49 | RCINCQKPLCPEEKQRHLDKKQRFHN | E6 |
| | IRGRWT | |
| 50 | DKKQRFHNIRGRWTGRCMSCCRSSRT | E6 |
| | RRETQL | |

What is claimed is:

1. A method for treating a human papilloma virus (HPV)-associated cancer in an individual, the method comprising:
administering an effective amount of a composition comprising peripheral blood mononuclear cells (PBMCs) to the individual, wherein the PBMCs comprise at least one HPV antigen delivered intracellularly, and
administering an effective amount of an antagonist of CTLA-4 and/or an antagonist of PD-1/PD-L1 to the individual.

2. The method of claim 1, wherein the antagonist of CTLA4 is an antibody that binds CTLA4.

3. The method of claim 1 or 2, wherein the antagonist of PD-1/PD-L1 is an antibody that binds PD-1 or an antibody that binds PD-L1.

4. The method of any one of claims 1-3, wherein an antibody that binds CTLA-4 and an antibody that binds PD-1 are administered to the individual.

5. The method of any one of claims 1-3, wherein an antibody that binds CTLA-4 is administered to the individual and an antibody that binds PD-L1 is administered to the individual.

6. The method of any one of claims 2-5, wherein the antibody that binds CTLA-4 is ipilimumab.

7. The method of any one of claims 3, 4 and 6, wherein the antibody that binds PD-1 is nivolumab.

8. The method of any one of claims 3, 4 and 6, wherein the antibody that binds PD-1 is pembrolizumab.

9. The method of any one of claims 3, 4 and 6, wherein the antibody that binds PD-L1 is atezolizumab.

10. A method for treating a HPV⁺ recurrent, locally advanced or metastatic tumor in an individual, the method comprising administering an effective amount of a composition comprising peripheral blood mononuclear cells (PBMCs) to the individual, wherein the PBMCs comprise at least one HPV antigen delivered intracellularly.

11. The method of claim 10, wherein the composition comprising PBMCs is administered in conjunction with one or more immune checkpoint inhibitors.

12. The method of claim 11, wherein the checkpoint inhibitor is an antagonist of CTLA-4 and/or an antagonist of PD-1/PD-L1 to the individual.

13. The method of claim 11 or 12, wherein the one or more immune checkpoint inhibitor is an antibody that binds PD-L1, CTLA-4, or PD-1.

14. The method of any or of claims 11-13, wherein the composition comprising PBMCs is administered in conjunction with an antibody that binds CTLA-4 and an antibody that binds PD-1.

15. The method of claim 13, wherein the antibody that binds PD-L1 is atezolizumab.

16. The method of any one of claims 13-15, wherein the antibody that binds CTLA-4 is ipilimumab.

17. The method of any one of claims 13, 14 and 16, wherein the antibody that binds PD-1 is nivolumab.

18. The method of any one of claims 13, 14 and 16, wherein the antibody that binds PD-1 is pembrolizumab.

19. The method of any one of claims 1-18, wherein the at least one HPV antigen is an HPV-16 antigen or an HPV-18 antigen.

20. The method of claim 19, wherein the at least one HPV antigen comprises a peptide derived from HPV E6 and/or E7.

21. The method of any one of claims 1-20, wherein the at least one HPV antigen comprises an HLA-A2-restricted peptide derived from HPV E6 and/or E7.

22. The method of claim 21, wherein the HLA-A2-restricted peptide comprises the amino acid sequence of any one of SEQ ID NOs:1-4.

23. The method of any one of claims 1-20, wherein the at least one HPV antigen comprises the amino acid sequence of any one of SEQ ID NOs:18-25.

24. The method on any one of claims 1-23, wherein the PBMCs comprise an antigen comprising the amino acid sequence of SEQ ID NO:19 and an antigen comprising the amino acid sequence of SEQ ID NO:23.

25. The method of any one of claims 1-24, where the individual is human.

26. The method of any one of claims 1-25, wherein the individual is positive for HLA-A*02.

27. The method of any one of claims 1-26, wherein the PBMCs are positive for HLA-A*02.

28. The method of any one of claims 1-27, where the PBMCs are autologous to the individual.

29. The method of any one of claims 1-28, wherein the individual is positive for human immunodeficiency virus (HIV).

30. The method of any one of claims 1-29, wherein the HPV-associated cancer is head and neck cancer, cervical cancer, anal cancer or esophageal cancer.

31. The method of any one of claims 1-30, wherein the composition comprising PBMCs are administered intravenously.

32. The method of any one of claims 1-9 and 12-31, wherein the antagonist of CTLA-4 and/or antagonist of PD-1/PD-L1 is administered intravenously, orally, or subcutaneously.

33. The method of any one of claims 2-9 and 13-32, wherein the antibody that binds CTLA-4 and/or the antibody that binds PD-1 and/or the antibody that binds PD-L1 is administered intravenously.

34. The method of any one of claims 1-33, wherein the effective amount of PBMCs comprising the at least one HPV antigen is about 0.5×10⁶ cells/kg to about 5.0×10⁶ cells/kg.

35. The method of any one of claims 6-9 and 16-34, wherein the effective amount of ipilimumab is about 1 mg/kg to about 3 mg/kg.

36. The method of any one of claims 7 and 17-35, wherein the effective amount of nivolumab is about 360 mg.

37. The method of any one of claims 9, 15, 16, and 19-36, wherein the effective amount of atezolizumab is about 1200 mg.

38. The method of any one of claims 1-37, wherein the composition comprising the PBMCs is delivered on day 1 of a three-week cycle.

39. The method of any one of claims 1-38, wherein the composition comprising the PBMCs is further administered on day 2 of a first three-week cycle.

40. The method of claim 38 or 39, wherein about 0.5×10⁶ cells/kg, about 2.5×10⁶ cells/kg about 5.0×10⁶ cells/kg are administered on day 1 of each three-week cycle.

41. The method of claim 39 or 40, wherein about 0.5×10⁶ cells/kg, about 2.5×10⁶ cells/kg or about 5.0×10⁶ cells/kg are administered on day 2 of the first three-week cycle.

42. The method of any one of claims 2-9 and 13-41, wherein the antibody that binds CTLA-4 and/or the antibody that binds PD-1 and/or the antibody that binds PD-L1 is administered once per three-week cycle.

43. The method of any one of claims 38-42, wherein the antibody that binds CTLA-4 is administered on day 1 of each three-week cycle.

44. The method of any one of claims 38-42, wherein the antibody that binds CTLA-4 is administered once per two three-week cycles.

45. The method of any one of claims 42-44, wherein the antibody that binds CTLA-4 is ipilimumab, wherein the ipilimumab is administered at a dose of about 3 mg/kg.

46. The method of any one of claims 42-45, wherein the antibody that binds PD-1 is administered on day 8 of the first three-week cycle and day 1 of each subsequent cycle.

47. The method of claim 46, wherein the antibody that binds PD-1 is nivolumab, wherein the nivolumab is administered at a dose of about 360 mg.

48. The method of any one of claims 38-42, wherein the antibody that binds CTLA-4 is ipilimumab, wherein the ipilimumab is administered on day 1 of the first three-week cycle of two three-week cycles at a dose of about 1 mg/kg and the antibody that binds PD-1 is administered on day 8 of the first three-week cycle and day 1 of each subsequent cycle at a dose of about 360 mg.

49. The method of any one of claims 38-45, wherein the antibody that binds PD-L1 is administered on day 8 of the first three-week cycle and day 1 of each subsequent cycle.

50. The method of claim 48 or 49, wherein the antibody that binds PD-L1 is atezolizumab, wherein the atezolizumab is administered at a dose of about 1200 mg.

51. The method of any one of claims 1-49, wherein the composition comprising PBMCs is administered to the individual for at least about three months, six months, nine months or one year.

52. The method of any one of claims 1-51, wherein the composition comprising PBMCs comprises
a) about 5×10⁶ PBMCs to about 5×10⁷ PBMCs,
b) cryopreservation medium at a percentage of about 40% to about 60% (w/w),
c) hypothermic preservation medium at a percentage of about 25% to about 35% (w/w), and
d) human serum albumin about 3% to about 8% (w/w),
wherein the pH of the formulation is about pH 6.0 to about pH 8.5.

53. The method of any one of claims 1-51, wherein the composition comprising PBMCs comprises
a) about 1×10⁶ PBMCs/mL to about 1×10⁷ PBMCs/mL,
b) cryopreservation medium at a at a percentage of about 40% to about 60% (w/w),
c) hypothermic preservation medium at a percentage of about 25% to about 35% (w/w), and
d) human serum albumin at a percentage of about 3% to about 8% (w/w),
wherein the pH of the formulation is about pH 6.0 to about pH 8.5.

54. The method of any one of claims 1-52, wherein the composition comprising PBMCs comprises
a) about 2.75×10⁷ PBMCs,
b) cryopreservation medium at a percentage of about 50% (w/w),
c) hypothermic preservation medium at a percentage of about 30% (w/w), and
d) human serum albumin at a percentage of about 5% (w/w),
wherein the pH of the formulation is about pH 7.4.

55. The method of any one of claims 1-54, wherein the composition comprising PBMCs comprises
a) about 5×10⁶ PBMCs/mL,
b) cryopreservation medium at a percentage of about 50% (w/w),
c) hypothermic preservation medium at a percentage of about 30% (w/w), and
d) human serum albumin at a percentage of about 5% (w/w),
wherein the pH of the formulation is about pH 7.4.

56. The method of any one of claims 1-51, wherein the composition comprising PBMCs comprises
a) about 5×10⁶ PBMCs to about 5×10⁷ PBMCs,
b) cryopreservation medium at a percentage of about 65% to about 95% (w/w),
c) human serum albumin at a percentage of about 3% to about 8% (w/w),
wherein the pH of the formulation is about pH 6.0 to about pH 8.5.

57. The method of any one of claims 1-51, wherein the composition comprising PBMCs comprises
a) about 1×10⁶ PBMCs/mL to about 1×10⁷ PBMCs/mL,
b) cryopreservation medium at a percentage of about 65% to about 95% (w/w),
c) human serum albumin at a percentage of about 3% to about 8% (w/w),
wherein the pH of the formulation is about pH 6.0 to about pH 8.5.

58. The method of any one of claims 1-51, wherein the composition comprising PBMCs comprises
a) about 2.5×10⁷ PBMCs,
b) cryopreservation medium at a percentage of about 80% (w/w),
c) human serum albumin at a percentage of about 5% (w/w),
wherein the pH of the formulation is about pH 7.4.

59. The method of any one of claims 1-51, wherein the composition comprising PBMCs comprises
a) about 5×10⁶ PBMCs/mL,
b) cryopreservation medium at a percentage of about 80% (w/w),
c) human serum albumin at a percentage of about 5% (w/w),
wherein the pH of the formulation is about pH 7.4.

60. The method of any one of claims 52-59, wherein the cryopreservation medium is CryoStor® CS10.

61. The method of any one of claims 52-55, wherein the hypothermic preservation medium is HypoThermasol® FRS.

62. The method of any one of claims 1-61, wherein the PBMCs comprises two or more of T cells, B cells, NK cells or monocytes.

63. The method of any one of claims 1-62, wherein the PBMCs comprises T cells, B cells, NK cells and monocytes.

64. The method of any one of claims 1-63, wherein
(a) about 25% to about 80% of the PBMCs are T cells,
(b) about 1.5% to about 30% of the PBMCs are B cells,
(c) about 3.0% to about 20% of the PBMCs are NK cells, or
(d) about 4.0% to about 45% of the PBMCs are monocytes.

65. The method of any one of claims 1-64, wherein the PBMCs comprising the at least one HPV antigen are prepared by a process comprising:
a) passing a cell suspension comprising a population of input PBMCs through a cell-deforming constriction, wherein a diameter of the constriction is a function of a diameter of the input PBMCs in the suspension, thereby causing perturbations of the input PBMCs large enough for the at least one HPV antigen to pass through to form perturbed input PBMCs; and
b) incubating the population of perturbed input PBMCs with the at least one HPV antigen for a sufficient time to allow the antigen to enter the perturbed input PBMCs, thereby generating the PBMCs comprising the at least one HPV antigen.

66. The method of claim 65, wherein the diameter of the constriction is about 4.2 μm to about 6 μm or about 4.2 μm to about 4.8 μm.

67. The method of any one of claims 1-66, wherein the PBMCs comprising the at least one HPV antigen are conditioned.

68. The method of claim 67, wherein the PBMCs comprising the at least one HPV antigen are conditioned by a process comprising incubating the PBMCs with an adjuvant for about 2 hours to about 10 hours, about 3 hours to about 6 hours, or about 4 hours at about 37° C. for the PBMCs to condition.

69. The method of claim 68, wherein the adjuvant is a CpG oligodeoxynucleotide (ODN), LPS, IFN-α, STING agonists, RIG-I agonists, poly I:C, R837, R848, a TLR3 agonist, a TLR4 agonist or a TLR 9 agonist.

70. The method of claim 68 or 69, wherein the adjuvant is a CpG 7909 oligodeoxynucleotide (ODN).


SEQUENCE LISTING (50 sequences)

SEQ ID NO: 1 - protein, 10 aa, Human papilloma virus
TIHDIILECV

SEQ ID NO: 2 - protein, 10 aa, Human papilloma virus
EVYDFAFRDL

SEQ ID NO: 3 - protein, 10 aa, Human papilloma virus
YMLDLQPETT

SEQ ID NO: 4 - protein, 9 aa, Human papilloma virus
RAHYNIVTF

SEQ ID NO: 5 - protein, 10 aa, Human papilloma virus
LPQLSTELQT

SEQ ID NO: 6 - protein, 8 aa, Human papilloma virus
QLCTELQT

SEQ ID NO: 7 - protein, 7 aa, Human papilloma virus
KQQLLRR

SEQ ID NO: 8 - protein, 10 aa, Human papilloma virus
VYSKQQLLRR

SEQ ID NO: 9 - protein, 10 aa, Human papilloma virus
MHGDTPTLHE

SEQ ID NO: 10 - protein, 6 aa, Human papilloma virus
GQAEPD

SEQ ID NO: 11 - protein, 16 aa, Human papilloma virus
YSKQQLLRREVYDFAF

SEQ ID NO: 12 - protein, 7 aa, Human papilloma virus
YCKQQLL

SEQ ID NO: 13 - protein, 8 aa, Human papilloma virus
CIVYRDGN

SEQ ID NO: 14 - protein, 15 aa, Human papilloma virus
SIVYRDGNPYAVSDK

SEQ ID NO: 15 - protein, 15 aa, Human papilloma virus
DLYCYEQLNDSSEEE

SEQ ID NO: 16 - protein, 20 aa, Human papilloma virus
CCKCDSTLRLCVQSTHVDIR

SEQ ID NO: 17 - protein, 20 aa, Human papilloma virus
SSKSDSTLRLSVQSTHVDIR

SEQ ID NO: 18 - protein, 36 aa, Artificial Sequence
  Synthetic Construct
LPQLSTELQTTIHDIILECVYSKQQLLRREVYDFAF

SEQ ID NO: 19 - protein, 25 aa, Artificial Sequence
  Synthetic Construct
QLCTELQTTIHDIILECVYCKQQLL

SEQ ID NO: 20 - protein, 25 aa, Artificial Sequence
  Synthetic Construct
KQQLLRREVYDFAFRDLCIVYRDGN

SEQ ID NO: 21 - protein, 35 aa, Artificial Sequence
  Synthetic Construct
VYSKQQLLRREVYDFAFRDLSIVYRDGNPYAVSDK

SEQ ID NO: 22 - protein, 35 aa, Artificial Sequence
  Synthetic Construct
MHGDTPTLHEYMLDLQPETTDLYCYEQLNDSSEEE

SEQ ID NO: 23 - protein, 25 aa, Artificial Sequence
  Synthetic Construct
QLCTELQTYMLDLQPETTYCKQQLL

SEQ ID NO: 24 - protein, 35 aa, Artificial Sequence
  Synthetic Construct
GQAEPDRAHYNIVTFCCKCDSTLRLCVQSTHVDIR

SEQ ID NO: 25 - protein, 35 aa, Artificial Sequence
  Synthetic Construct
GQAEPDRAHYNIVTFSSKSDSTLRLSVQSTHVDIR

SEQ ID NO: 26 - DNA, 20 bp, Artificial Sequence
  Synthetic Construct
ggggtcaacgttgagggggg

SEQ ID NO: 27 - DNA, 20 bp, Artificial Sequence
  Synthetic Construct
gggggacgatcgtcgggggg

SEQ ID NO: 28 - DNA, 21 bp, Artificial Sequence
  Synthetic Construct
ggggacgacgtcgtggggggg

SEQ ID NO: 29 - DNA, 20 bp, Artificial Sequence
  Synthetic Construct
tccatgacgttcctgatgct

SEQ ID NO: 30 - DNA, 20 bp, Artificial Sequence
  Synthetic Construct
tccatgacgttcctgacgtt

SEQ ID NO: 31 - DNA, 24 bp, Artificial Sequence
  Synthetic Construct
tcgtcgttttgtcgttttgtcgtt

SEQ ID NO: 32 - DNA, 22 bp, Artificial Sequence
  Synthetic Construct
tcgtcgttgtcgttttgtcgtt

SEQ ID NO: 33 - DNA, 23 bp, Artificial Sequence
  Synthetic Construct
tcgacgttcgtcgttcgtcgttc

SEQ ID NO: 34 - DNA, 26 bp, Artificial Sequence
  Synthetic Construct
tcgcgacgttcgcccgacgttcggta

SEQ ID NO: 35 - DNA, 22 bp, Artificial Sequence
  Synthetic Construct
tcgtcgttttcggcgcgcgccg

SEQ ID NO: 36 - DNA, 25 bp, Artificial Sequence
  Synthetic Construct
tcgtcgtcgttcgaacgacgttgat

SEQ ID NO: 37 - DNA, 29 bp, Artificial Sequence
  Synthetic Construct
tcgcgaacgttcgccgcgttcgaacgcgg

SEQ ID NO: 38 - protein, 35 aa, Artificial Sequence; identical to SEQ ID NO: 22
  Synthetic Construct

SEQ ID NO: 39 - protein, 35 aa, Artificial Sequence
  Synthetic Construct
LYCYEQLNDSSEEEDEIDGPAGQAEPDRAHYNIVT

SEQ ID NO: 40 - protein, 35 aa, Artificial Sequence; identical to SEQ ID NO: 24
  Synthetic Construct

SEQ ID NO: 41 - protein, 35 aa, Artificial Sequence
  Synthetic Construct
TLRLCVQSTHVDIRTLEDLLMGTLGIVCPICSQKP

SEQ ID NO: 42 - protein, 32 aa, Artificial Sequence
  Synthetic Construct
MHQKRTAMFQDPQERPRKLPQLCTELQTTIHD

SEQ ID NO: 43 - protein, 32 aa, Artificial Sequence
  Synthetic Construct
LPQLCTELQTTIHDIILECVYCKQQLLRREVY

SEQ ID NO: 44 - protein, 25 aa, Artificial Sequence
  Synthetic Construct
KQQLLRREVYDFAFRDLCIVYRDGN

SEQ ID NO: 45 - protein, 26 aa, Artificial Sequence
  Synthetic Construct
RDLCIVYRDGNPYAVCDKCLKFYSKI

SEQ ID NO: 46 - protein, 25 aa, Artificial Sequence
  Synthetic Construct
DKCLKFYSKISEYRHYCYSLYGTTL

SEQ ID NO: 47 - protein, 25 aa, Artificial Sequence
  Synthetic Construct
HYCYSLYGTTLEQQYNKPLCDLLIR

SEQ ID NO: 48 - protein, 32 aa, Artificial Sequence
  Synthetic Construct
YGTTLEQQYNKPLCDLLIRCINCQKPLCPEEK

SEQ ID NO: 49 - protein, 32 aa, Artificial Sequence
  Synthetic Construct
RCINCQKPLCPEEKQRHLDKKQRFHNIRGRWT

SEQ ID NO: 50 - protein, 32 aa, Artificial Sequence
  Synthetic Construct
DKKQRFHNIRGRWTGRCMSCCRSSRTRRETQL